(19) United States
(12) Patent Application Publication
(10) Pub. No.: US 2009/0324628 A1
(43) Pub. Date: Dec. 31, 2009

(54) COMPOUNDS USEFUL IN THE DIAGNOSIS AND TREATMENT OF MALARIA

(75) Inventors: Thor Grundtvig Theander, Ishoej (DK); Thomas Lavstsen, Horsholm (DK); Lars Hviid, Copenhagen (DK); Morten A. Nielsen, Copenhagen (DK); Pamela A. Magistrado, Tanga Region (TZ); Ali Salanti, Bronshoj (DK); Trine Staalso, Copenhagen (DK); Anja Tatiana Ramstedt Jensen, Bronshoj (DK); Louise Jorgensen, Horsholm (DK)

Correspondence Address: SEATTLE BIOMEDICAL RESEARCH INSTITUTE;ATTN: KAREN BLOCHLINGER, 307 WESTLAKE AVENUE N., SUITE 500, SEATTLE, WA, 98109-5219, US

(73) Assignee: KOBENHAVNS UNIVERSITET PANIUM, Copenhagen N (DK)

(21) Appl. No.: 10/585,027

(22) PCT Filed: Dec. 28, 2004
(86) PCT No.: PCT/DK2004/000922

§ 371 Date: Sep. 15, 2009

(30) Foreign Application Priority Data
Dec. 30, 2003 (DK) PA 2003 01954

(51) Int. Cl. A61K 39/00; A61P 37/04

(52) U.S. Cl. 424/185.1

(57) ABSTRACT

The present invention relates to nucleic acid molecules related to the PFD1235w/MAL7P1.1, PF11_0008, and PF13_0003 gene families as well as amino acid sequences encoded by such nucleic acid molecules with respect to their role in mediating adhesion of infected red blood cells to endothelial cells, which is characteristic for the pathogenesis of severe malaria (SM). Accordingly, the invention provides pharmaceutical compositions and vaccines, hereunder nucleotide-based vaccines comprising compounds that are related to VAR4, VAR5, and/or VAR6 polypeptides and PFD1235w/MAL7P1.1 PF11_0008, and/or PF13_0003 nucleic acid molecules. The invention further relates to the use of these compounds as medicaments and for the manufacture of compositions, such as immunogenic compositions. In addition, the invention relates to methods of treatment and prevention of severe malaria wherein these methods are based on the nucleic acid molecules and polypeptides of the invention. As these compounds can also be used as biotechnological tools the invention provides in vitro diagnostic methods and kits comprising reagents and IgGs/antibodies designated to the use in such methods. The invention also relates to methods of identifying agents capable of modulating the VAR4, VAR5, and/or VAR6 dependent adhesion to endothelial cells and agent capable of interacting with VAR4, VAR5, and/or VAR6. Finally, a method for identifying polypeptides, which will induce a specific IgG/antibody response upon administration to a subject is provided by the invention.

### FIELD OF THE INVENTION

The present invention relates to the fields of preventing or treating malaria and it provides compounds, which are useful within these fields. These compounds may constitute parts of pharmaceutical compositions and vaccines and be used in methods of treatment, as medicaments and for the manufacture of compositions and/or these compounds may provide basis for a method of generating a vaccine against malaria. Furthermore, the invention relates to the use of these compounds as biotechnological tools and in in vitro diagnostic methods and kits.

### GENERAL BACKGROUND

Malaria constitutes a permanent catastrophe. Annually, the disease kills between 1 and 2 million Africans and the economic losses due to malaria constitute a hindrance for economic development. In areas of stable malaria transmission the disease mainly affects children, because adults have acquired immunity. In these areas immunity to severe malaria and protection against malaria deaths is acquired early in life after a few clinical infections, whereas the acquisition of immunity that protect individuals from uncomplicated febrile malaria episodes is a much more sluggish process, requiring years of exposure. Thus, the epidemiological data indicate that immunity against severe disease is mediated through a different mechanism than immunity against uncomplicated disease, and that the targets on the parasites for these two kinds of malaria immunity are separate.

Malaria is caused by unicellular parasites living and multiplying asexually in the red blood cells (RBC). In each 48-hour cycle, the parasites invade RBC, multiply within them, and eventually burst them, before they go on to invade new RBC.

Plasmodium falciparum is the most virulent of the four species causing malaria and responsible for most malarial deaths. The particular virulence of P. falciparum is due to the ability of infected erythrocytes to adhere to a variety of host receptors on the endothelial lining such as ICAM-1, VCAM, thrombospondin, ELAM-1, and CD36, and avoid splenic clearance. Unchecked growth and the accumulation of sequestered parasites in vital organs such as the brain are crucial elements in the pathogenesis of severe malaria.

Sequestration is mediated through parasite-encoded, clonally variant surface antigens (VSA) inserted into the membrane of the infected RBC and is thought to be an immune evasion strategy evolved to avoid splenic clearance. The VSA expressed on infected erythrocytes can be divided into serological types using plasma from individuals living in malaria endemic areas as typing reagents.

By flow cytometry analyses, two main serotypes have been defined, one, VSA_{SM} is mainly expressed by parasites causing severe and life-threatening P. falciparum malaria, and the other, VSA_{UM} is dominantly expressed during uncomplicated malaria infections in semi-immune individuals.

Naturally acquired immunity to malaria is mediated by plasma IgG, which control the growth of P. falciparum. VSAs are probably the main target of these antibodies, as acquisition of protection from P. falciparum malaria corresponds to a gradual accumulation of IgG with a broad range of VSA-specificities. Furthermore, VSA-specific immune responses steadily restrict the repertoire of VSA that are compatible with parasite survival, and drive VSA expression away from VSA_{SM} towards VSA_{UM} (Nielsen et al., 2002). VSA expression therefore is non-random as it depends on the degree of immunity in the infected host.

The best-characterised VSA are encoded by the var genes. This gene family, encompassing about 60 members per genome, encodes a highly polymorphic set of variant proteins, which collectively have been named P. falciparum erythrocyte membrane-protein 1 (PfEMP1). These proteins are located on the surface of the P. falciparum-infected erythrocytes and have been shown to mediate adhesion to a number of host receptors.

The var genes contain a large variable 5′ exon and a more conserved 3′ exon separated by an intron. The 5′ exon normally contains one or two Cysteine-rich Inter-Domain Regions (CIDR) and from two to seven domains designated DBL after their similarity to the Duffy Binding Ligand of P. vivax (Smith et al. 2000). A given parasite expresses only one PfEMP1 at a time, but in each generation a fraction of the daughter parasites may switch to expression of alternative PfEMP1 species through an unknown process. Different PfEMP1 molecules have different receptor specificities, and clonal switching between expression of the various var gene products in a mutually exclusive manner allows the parasite to modify its adhesion properties (Wahlgren et al., 1999).

Thus, the currently available data indicate that severe malaria is caused by parasites expressing a subset of PfEMP1 molecules and that antibodies directed against these are responsible for the protection against severe disease acquired early in life by children in endemic areas. It follows that the specific identification of the particular PfEMP1 molecules expressed by the parasites causing severe malaria is important for malaria vaccine development.

The obvious strategies followed in many laboratories are either to attempt to identify the var gene that is dominantly transcribed or to determine which PfEMP1 is expressed by parasites isolated from patients suffering from severe malaria.

Both strategies have been halted because the var genes show extensive intra- and inter-genomic variation.

Attempts to identify VSA_{SM}-type var gene transcription has been foiled because primer bias and concomitant transcription of several var genes makes it is impossible to quantify and compare transcription of these genes, when all the target nucleic acid sequences are not known. The proteomic approach has proved difficult, partly due to the variation in the repertoire already mentioned, partly because PfEMP1 is expressed at low levels on the surface of erythrocytes, and it has been difficult to obtain sufficient amounts of protein for a reliable mass spectrometry approach.

Frustrated by these efforts several laboratories has tried to identify the most relevant PfEMP1 by generating parasite lines with known adhesion phenotype using repeated rounds of panning and identifying the dominantly transcribed var genes. This approach has identified PfEMP1 molecules with a known receptor affinity, but since parasites causing severe malaria have never been shown to possess a common binding phenotype, the relevance of these PfEMP1 molecules in the pathogenesis of severe malaria is unclear.

The PfEMP1s constitute a large and polymorphic family. The proteins described in this application serve a unique function for the parasite in providing high growths rates in non-immune individuals and renders these parasites a unique serological phenotype. This discovery is of great potential importance because it makes it possible to design strategies that will specifically aim at reducing malaria deaths by preventing, diagnosing, and treating severe malaria.

The entire P. falciparum genome of one parasite isolate, 3D7, have previously been data-mined using bioinformatic tools. Based on both coding- and non-coding regions var genes have been found to group into three major groups (group A, B and C) and two intermediate groups B/A and B/C representing transitions between the three major groups. Group A consists of ten genes consistently identified as a distinct group by sequence analysis (Lavstsen et al. 2003).

Although the proposed grouping of var genes in 3D7 is common knowledge to a person skilled in the art and the sequence of the entire 3D7 P. falciparum genome is known as sequence submissions, information on function of SEQ ID 2, SEQ ID NO.: 4, and SEQ ID NO.: 6 in the pathogenesis of malaria and their relevance for a vaccine against severe malaria has not previously been described.

In the 3D7 isolate, Group A contains 10 var genes of which we have identified three var genes, using a novel method, as being functionally relevant for development of a vaccine against severe malaria. However, the global repertoire of var genes is unknown, but estimated to encode more than 1027 possible variants PfEMP1.

### SUMMARY OF THE INVENTION

In essence, the inventive concept described herein is based on the observation that three genes, PFD1235w/MAL7P1.1, PF11_{—}0008, and PF13_{—}0003 (referred to as SEQ ID NO.: 1, SEQ ID NO.: 3, and SEQ ID NO.: 5) are transcriptionally up-regulated in parasites of the species Plasmodium falciparum, when these parasites have been selected for increased antibody recognition by a novel selection method, described in the present application, using plasma from semi-immune children. This selection conway parasites a unique phenotype characteristic of parasites causing severe malaria. This phenotype is also obtained when parasites are selected for adhesion to a special kind of bone marrow derived endothelical cells and also results in the up-regulation of SEQ ID NO.: 1, SEQ ID NO.: 3, and SEQ ID NO.: 5. As the cytoadhesion to human endothelial cells and the antibody recognition of infected RBC is intimately linked to severe malaria, products of these genes provide for novel approaches to diagnosing and treating malaria prophylactically and/or therapeutically.

In the broadest sense, the present invention relates to the polypeptides, VAR4, VAR5, and VAR6 (referred to as SEQ ID NO.: 2, SEQ ID NO.: 4, and SEQ ID NO.: 6) encoded by SEQ ID NO.: 1, SEQ ID NO.: 3, and SEQ ID NO.: 5, respectively or parts hereof as well as polypeptides, which with respect to their sequence are identical in part to such sequences. In addition, the invention relates to the SEQ ID NO.: 1, SEQ ID NO.: 3, and SEQ ID NO.: 5 nucleic acid molecule or parts hereof as well as nucleic acid molecules, which with respect to their sequence are identical in part to such sequences.

A presently preferred embodiment relates to medical uses of any of the polypeptides and/or nucleic acids according to the present invention as well as methods of treatment of malaria comprising molecules of the invention.

Other aspects of the invention include pharmaceutical compositions and vaccines based on the molecules of the invention. In addition, the invention comprises polypeptides or nucleic acid molecules of the invention as medicaments, and the use of these polypeptides and nucleic acids for the manufacture of compositions, hereunder-immunogenic compositions which are to administered in order to prophylactically or therapeutically reduce the incidence, prevalence or severity of malaria, especially severe malaria.

It is further within the scope of the present invention to provide a method of treatment and prevention of malaria, which comprises administering an effective amount of one or more of the described molecules of the invention to a subject.

It will appear that the mentioned polypeptides and nucleic acid molecules will also be useful as biotechnological tools. Therefore, the invention also relates to in vitro diagnostic methods, which comprise contacting a sample with polypeptides or nucleic acid molecules having the sequences described above, allowing in vitro reactions to occur and subsequently detecting any molecular complexes formed. These may for instance be complexes of antigens and antibodies. In some aspects of the invention, the polypeptides of the invention are parts of diagnostic kits. Alternatively, these kits may comprise antibodies, which specifically recognise such polypeptides. Kits may also compromise, but are not limited to oligonucleotides as part of diagnostic kits based on techniques such as array, PCR, and real-time quantitative PCR.

### DETAILED DESCRIPTION OF THE INVENTION

### The New Strategy

The present inventors have taken a novel approach and based their strategy on the hereby disclosed knowledge of the serological phenotype expressed by parasites causing severe malaria (VSA_{SM}) and the availability of all var genes sequences in the parasite line 3D7.

In culture 3D7 will normally express a typical VSA_{UM} serotype, thus the present inventors first developed a method to generate VSA_{SM} expressing 3D7 lines and then used quantitative real time PCR to identify the dominant var gene transcripts.

They hereafter made specific probes and antibodies, that allowed them to identify the PfEMP1 molecules expressed on the surface of the erythrocytes responsible for the serological phenotype carried by the selected parasite line.

This serological phenotype was also obtained when the 3D7 parasite line was selected for adhesion to bone marrow derived endothelial cells.

The relevance of the discovered PfEMP1 molecules were further substantiated by the fact that parasites expressing the molecules have a high growth rate in non-immune individuals, and that var genes with a high similarity to the discovered genes can be identified in other parasites. Their approach is completely different from previous strategies of defining variant molecules responsible for binding to different receptors.

The strategy is based on both epidemiological studies showing that immunity to severe malaria is acquired more rapidly than protection from uncomplicated disease and sub-clinical infection findings indicating that VSA_{SM} constitute a restricted and antigenically conserved VSA subset, whereas VSA_{UM} are more diverse.

Together, these observations enabled developing of the disease-ameliorating strategies presented in the present application, which can protect against mortality and severe morbidity by accelerating acquisition of immunity to VSA_{SM}-expressing parasites and thereby forcing VSA expression away from VSA_{SM}.

As highlighted earlier Plasmodium falciparum erythrocyte membrane protein-1 (PfEMP1), are a highly polymorphic and diverse family of proteins. Every parasite genome carries about 60 genes encoding PfEMP1 and the repertoire of PfEMP1 genes differ from parasite genome to parasite genome. Thus, PfEMP1 genes show both intra- and inter-genomic variation, and the global repertoire of PfEMP1 proteins is unknown, but estimated to be very large, in fact the present inventors have estimated that there are more 1027 possible variants of this protein. The common features shared by the PfEMP1 family of genes and proteins are the organization of the genes (two exons and an intron), and the presence of domain structures that can be classified as Duffy Binding Ligand-like (DBL) or Cysteine-Rich Interdomain Region (CIDR).

In addition, the proteins share a relative conserved C-terminal tail consisting of a trans-membrane region and a relatively short intracellular domain. However, it must be stressed that the genes and the encoded proteins vary considerably between each other; both with regards to sequence (primary structure) and organization of the domains (Lavstsen et al., 2003).

It is also clear that expression of different PfEMP1 molecules confer parasite different functional (Smith et al., 2000; Robinson et al., 2003) and antigenic characteristics (Salanti et al., 2003). Within PfEMP1 domains classified as belonging to the same group and subgroup (i.e. DBLα, DBLβ, CIDRγ etc) short identity blocks of 2-14 amino acids can be identified between hyper variable blocks of varying lengths (of up to several hundred amino acids) in which there is no or very little homology between randomly chosen PfEMP1. Thus, PfEMP1 molecules constitute a very large and diverse family of proteins.

### Inventive Concept

The inventive concept disclosed in the present application is based on the unexpected observation that the transcription of 3 specific Plasmodium falciparum var genes, SEQ ID NO.: 1, SEQ ID NO.: 3, and SEQ ID NO.: 5, all members of Group A var genes, is up-regulated in parasite lines and clones carrying the VSA_{SM} phenotype. This up-regulation followed both after antibody-selection and selection for adhesion of infected RBC to bone marrow derived endothelial cells in vitro.

These observations shows that the proteins encoded by these three specific var genes are responsible for inducing the first IgG with specificity to PfEMP1 on iRBC and thus the dominant PfEMP1 expressed during P. falciparum infections in immunologically naïve individuals. Such proteins are useful as therapeutic and prophylactic agents as well as biological tools and diagnostic agents for the study, treatment and prevention of malaria, since these proteins described serve a unique function for the parasite in providing high growths rates in non-immune individuals and renders these parasites a unique serological phenotype. This is of great potential importance because it makes it possible to design strategies that will specifically aim at reducing malaria deaths by preventing, diagnosing, and treating malaria.

### Polypeptide Molecules of the Invention

Thus, in its broadest aspect, the present invention relates to these 3 isolated polypeptides comprising at least one amino acid sequence selected from the group consisting of at least one of
a) SEQ ID NO.: 2, SEQ ID NO.: 4 or SEQ ID NO.: 6, and
b) a sequence having at least 80% sequence identity to a), and
c) sub-sequences of a) or b) with a minimum length of 10 amino acids, and
d) sub-sequences of a) b) comprising at least one B-cell epitope;

With the proviso that the Exon 2 of SEQ ID NO.: 2, SEQ ID NO.: 4 and/or SEQ ID NO.: 6 is excluded.

With the proviso that the “Fragment 1” and/or “Fragment 2” and/or “Fragment 3” and/or “Fragment 4” and/or “Fragment 5” and/or “Fragment 6” and/or “Fragment 7” and/or “Fragment 8” and/or “Fragment 9” and/or “Fragment 10” and/or “Fragment 11” and/or “Fragment 12” and/or “Fragment 13” and/or “Fragment 14” and/or “Fragment 15” and/or “Fragment 16” and/or “Fragment 17” “Fragment 18” and/or “Fragment 19” and/or “Fragment 20” and/or “Fragment 21” and/or “Fragment 22” and/or “Fragment 23” and/or “Fragment 24” and/or “Fragment 25” and/or “Fragment 26” of SEQ ID NO.: 2, SEQ ID NO.: 4, and/or SEQ ID NO.: 6 is excluded.

With the proviso that the “Fragment 27” and/or “Fragment 28” and/or “Fragment 29” and/or “Fragment 30” and/or of SEQ ID NO.: 2 and/or SEQ ID NO.: 6 is excluded.

With the proviso that the “Fragment 31” and/or “Fragment 32” of SEQ ID NO.: 2 is excluded.

With the proviso that the amino acid sequence of SEQ ID NO.: 2, SEQ ID NO.: 4 and/or SEQ ID NO.: 6 is excluded.

For the present and any of the following aspects of the invention it applies that it is an object of preferred embodiments of the present invention to provide polypeptides, which are subject to antibody recognition by antibodies in sera from young children living in areas of high malaria transmission intensity and/or capable of mediating cyto-adhesion of intact erythrocyte infected by a parasite to endothelial cells, but not the CD36 receptor. The distinction to CD36 adhesion can be evaluated by the examples of the present invention, e.g. as described in example 4.

### Length of the Molecules

For all the aspects of the invention, it is apparent that the polypeptides of the invention, which form the basis of the described embodiments of the invention may be less or equal to any length between 6-3552 (SEQ ID NO.: 2), 6-2992 (SEQ ID NO.: 4) and 6-3344 (SEQ ID NO.: 6) amino acids, since these polypeptide all have great immunogenic capabilities and thus are especially great as candidates for the development for the treatment of malaria, particularly in the development of a vaccine.

Thus the present invention relates to any sub-sequence originating from any of the polypeptides having an amino acid length such as but not limited to less than or equal to 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 350, 400, 450, 500, 600, 700, 800, 900, 1000, 1250, 1500, 1750, 2000, 2250, 2500, 2750, or 2990 amino acids in length.

In a further embodiment the length of a sub-sequence of SEQ ID NO.: 2 can be less than or equal to 3000, 3100, 3200, 3300, 3400, 3500 or 3550 amino acids in length, whereas the length of a sub-sequence of SEQ ID NO.: 6 furthermore can be less than or equal to 3000, 3100, 3200, 3300 or 3340 amino acids in length.

In addition to these sequences, fragments or sub-sequences of the polypeptide of the invention, larger proteins/polypeptides comprising such sub-sequences as part of their sequence, are also embodiments of the present invention, thus with respect to all aspects of the invention it may be preferred that the polypeptides of the invention may have a length above the disclosed full length of 3553 (SEQ ID NO.: 2), 2994 (SEQ ID NO.: 4) or 3346 (SEQ ID NO.: 6) amino acids, if combined with other amino acids for e.g. preparation purposes.

Some characteristic lie within the polypeptide sequence of SEQ ID NO.: 2, SEQ ID NO.: 4, and SEQ ID NO.: 6 such as B- and T-cell epitopes.

T-cell epitopes were defined using the SYFPEITHI server at Centre for Biological Sequence Analysis BioCentrum-DTU at the Technical University of Denmark (http://syfpeithi.bmi-heidelberg.com/Scriots/MHCServer.dll/EpPredict.htm) with default settings. B-cell epitopes were defined using the Protean 4.0 software in the DNAstar package with default settings. For SEQ ID NO.: 2 predicted T-cell epitopes are defined as, but not limited to amino acid no. 13-45, 93-107, 135-167, 201-236, 247-261, 302-316, 327-348, 394-409, 432-446, 457-473, 532-546, 573-589, 595-609, 625-639, 678-692, 870-890, 942-956, 986-1000, 1097-1111, 1130-1144, 1151-1165, 1196-1210, 1473-1492, 1508-1548, 1593-1630, 1633-1647, 1800-1814, 1830-1844, 1923-1937, 1943-1957, 1997-2025, 2235-2249, 2322-2336, 2378-2404, 2427-2441, 2464-2478, 2507-2521, 2533-2553, 2608-2637, 26662-2683, 2693-2720, 2794-2808, 2851-2866, 2879-2904, 2965-2979, 3074-3088, 3092-3149, 3201-3216, 3236-3250, 3274-3288, 3297-3311, 3322-3336, 3350-3364, 3375-3395, 3488-3502, 3530-3544.

For SEQ ID NO.: 2 predicted B-cell epitopes are defined as, but not limited to amino acid no. 197-281, 365-470, 526-575, 631-772, 820-891, 905-933, 961-1003, 1024-1066, 1073-1188, 1206-1248, 1269-1367, 1402-1437, 1535-1731, 1991-2026, 2054-2096, 2124-2229, 2278-2313, 2404-2460, 2488-2530, 2663-2713, 2832-2965, 3014-3112, 3154-3210, 3322-3406.

For SEQ ID NO.: 4 predicted T-cell epitopes are defined as, but not limited to amino acid no. 117-131, 154-168, 177-213, 224-238, 258-272, 307-321, 332-346, 419-434, 454-468, 512-534, 561-595, 619-641, 645-680, 691-710, 781-795, 983-997, 1043-1057, 1065-1093, 1143-1185, 1296-1310, 1383-1397, 1453-1467, 1567-1597, 1653-1667, 1693-1707, 1740-1754, 1774-1836, 1855-1873, 1929-1943, 2066-2080, 2108-2122, 2130-2156, 2174-2188, 2206-2220, 2282-2296, 2309-2323, 2439-2453, 2552-2566, 2578-2592, 2625-2639, 2644-2673, 2697-2714, 2719-2733, 2744-2774, 2802-2816, 2918-2943, 2971-2985.

For SEQ ID NO.: 4 predicted B-cell epitopes are defined as, but not limited to amino acid no. 30-65, 89-113, 125-142, 213-272, 390-432, 491-514, 733-792, 833-851, 874-904, 1040-1069, 1128-1170, 1187-1211, 1258-1276, 1306-1365, 1394-1406, 1441-1499, 1542-1571, 1648-1678, 1884-1908, 1949-1979, 2268-2309, 2345.2369, 2392-2416, 2853-2906.

For SEQ ID NO.: 6 predicted T-cell epitopes are defined as, but not limited to amino acid no. 14-28, 43-57, 71-85, 222-248, 251-265, 301-315, 318-332, 359-373, 416-443, 446-460, 471-485, 518-532, 579-593, 624-643, 783-797, 949-967, 986-1000, 1016-1030, 1075-1093, 1141-1155, 1265-1279, 1340-1372, 1440-1471, 1505-1515, 1529-1556, 1634-1648, 1659-1673, 1687-1701, 1712-1726, 1813-1827, 1841-1855, 1875-1995, 2092-2110, 2137-2151, 2161-2171, 2249-2263, 2319-2333, 2379-2393, 2475-2495, 2502-2516, 2549-2564, 2591-2605, 2629-2643, 2650-2669, 2674-2688, 2734-2766, 2916-2930, 2989-3018, 3038-3052, 3115-3129, 3131-3145, 3167-3187, 3248-3262, 3323-3337.

For SEQ ID NO.: 6 predicted B-cell epitopes are defined as, but not limited to amino acid no. 7-27, 383-429, 627-786, 898-990, 1089-1221, 1307-1340, 1373-1525, 1670-1815, 1901-1934, 2007-2027, 2225-2251, 2363-2409, 2495-2528, 2614-2660, 2706-2911, 3221-3300.

Some characteristic structures lie within the polypeptide sequence of SEQ ID NO.: 2, SEQ ID NO.: 4, and SEQ ID NO.: 6 and therefore also within the nucleotide sequence encoding these polypeptide sequences. Such structures comprise, but are not necessarily limited to DBL2, DBL5, and C2.

Preferred embodiments of the present invention include specific sub-sequences of the polypeptide of the invention having a minimum length of 10 amino acids such as sub-sequences that are at least 100 amino acids long. In even more preferred embodiments of the invention, these sub-sequences can be shown by known molecular biological techniques to be involved in the interaction with endothelial receptors and/or to be recognised by antibodies using plasma from young semi-immune children.

It is anticipated that relatively short sequences within the SEQ ID NO.: 2, SEQ ID NO.: 4 and/or SEQ ID NO.: 6 are responsible for mediating adhesion to endothelial receptors other than CD36. In particular, it is possible that certain CIDR or DBL domains or parts hereof are responsible for the adhesion and/or for antibody recognition.

In other preferred embodiments of the invention, the sequences of the polypeptide of the invention can be shown to possess one or more antigen epitopes. In particular, such epitopes may be B-cell epitopes. Optionally, the sub-sequences may also comprise one or more T-cell epitopes alone or in combination with the B-cell epitopes. Finally, also larger polypeptides comprising the polypeptide of the invention or sub-sequences hereof with antigen epitopes and/or sequences involved in interaction with endothelial receptors are embodiments of the present invention.

It is also apparent that the polypeptide sequences of the invention can be present in the form of fusion proteins. In a further preferred embodiment, this fusion protein will comprise polypeptide sequences, which will facilitate the purification or detection of the protein. These polypeptide sequences may be but are not limited to tags that will facilitate purification and detection using commercially available systems such as the HA-, -c-myc, His or GST tags.

The polypeptide embodiments of the present invention can therefore exhibit a vast degree of sequence identity to the full-length of SEQ ID NO.: 2, SEQ ID NO.: 4 and/or SEQ ID NO.: 6. It can for instance be appreciated that a fusion protein carrying within its sequence one or more B-cell epitopes and or regions of the polypeptide of the invention that are involved in adhesion to endothelial receptors will have a relatively low overall degree of sequence identity to full-length SEQ ID NO.: 2, SEQ ID NO.: 4 and/or SEQ ID NO.: 6.

Sequence Identity of the Polypeptide Molecules

For all the aspects of the invention, it is thus apparent that the polypeptides of the invention may include sequences, which show anywhere between 40-100% sequence identity, such as at least 41%, at least 42%, at least 43%, at least 44%, at least 45%, at least 460%, at least 47%, at least 48%, at least 49% at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90% at least 91%, at least 92%, at least 93%, at least 94%, at least 95%, at least 96%, at least 97%, at least 98%, at least 99% or preferably 1000% sequence identity to SEQ ID NO.: 2, SEQ ID NO.: 4 and/or SEQ ID NO.: 6 or a fragment or sub-sequence thereof.

As understood by the skilled adressee, the sequence identity of the polypeptide molecules of the present invention will be higher the smaller the fragments or sub-sequences in order to be unique, thus in some embodiments of the present invention, the fragments or sub-sequences are of 20-50 amino acid long and has a sequence identity of 80-100%, such as 20-50 amino acid long, 20-40 amino acid long, 20-30 amino acid long, 20-25 amino acid long, 30-50 amino acid long, 30-40 amino acid long, 30-35 amino acid long, 40-50 amino acid long, 40-45 or 45-50 amino acid long all with a sequence identity of 80-100%, such as 80% identical, 81% identical, 82% identical, 83% identical, 84% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical or even 100% identical.

In other embodiments of the present invention, the fragments or sub-sequences are of 51-750 amino acid long and has a sequence identity of 70-100%, such as 51-750 amino acid long, 60-700 amino acid long, 70-600 amino acid long, 80-500 amino acid long, 90-400 amino acid long, 100-250 amino acid long, 300-350 amino acid long, 400-500 amino acid long, 200-650 or 450-500 amino acid long all with a sequence identity of 70-100%, such as 70% identical, 71% identical, 72% identical, 73% identical, 74% identical, 75% identical, 80% identical, 85% identical, 88% identical, 89% identical, 90% identical, 91% identical, 92% identical, 93% identical, 94% identical, 95% identical, 96% identical, 97% identical, 98% identical, 99% identical or even 100% identical.

In yet other embodiments of the present invention, the fragments or sub-sequences are of more than 751 amino acid long and has a sequence identity of 60%-100%, such as more 751 amino acid long, more 800 amino acid long, more than 900 amino acid long, more than 1000 amino acid long, more than 1100 amino acid long, more than 1200 amino acid long, more than 1300 amino acid long, more than 1400 amino acid long, more than 2000 amino acids long or 2500 amino acid long all with a sequence identity of 60-100%, such as 60% identical, 61% identical, 62% identical, 63% identical, 64% identical, 65% identical, 70% identical, 75% identical, 77% identical, 80% identical, 85% identical, 88% identical, 92% identical, 93% identical, 94% identical, 95% identical, 96% identical, 97% identical, 98% identical, 99% identical or even 100% identical.

Additionally, variants are also an embodiment of the present invention and are determined based on a predetermined number of conservative amino acid substitutions as defined herein below. Conservative amino acid substitution as used herein relates to the substitution of one amino acid (within a predetermined group of amino acids) for another amino acid (within the same group), wherein the amino acids exhibit similar or substantially similar characteristics.

Within the meaning of the term “conservative amino acid substitution” as applied herein, one amino acid may be substituted for another within the groups of amino acids indicated herein below:

Amino acids having polar side chains (Asp, Glu, Lys, Arg, His, Asn, Gln, Ser, Thr, Tyr, and Cys,)
Amino acids having non-polar side chains (Gly, Ala, Val, Leu, Ile, Phe, Trp, Pro, and Met)
Amino acids having aliphatic side chains (Gly, Ala Val, Leu, Ile)
Amino acids having cyclic side chains (Phe, Tyr, Trp, His, Pro)
Amino acids having aromatic side chains (Phe, Tyr, Trp)
Amino acids having acidic side chains (Asp, Glu)
Amino acids having basic side chains (Lys, Arg, His)
Amino acids having amide side chains (Asn, Gln)
Amino acids having hydroxy side chains (Ser, Thr)
Amino acids having sulphor-containing side chains (Cys, Met),
Neutral, weakly hydrophobic amino acids (Pro, Ala, Gly, Ser, Thr)
Hydrophilic, acidic amino acids (Gln, Asn, Glu, Asp), and
Hydrophobic amino acids (Leu, Ile, Val)

Preferred conservative amino acids substitution groups are: valine-leucine-isoleucine, phenylalanine-tyrosine, lysine-arginine, alanine-valine, and asparagine-glutamine.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

The present invention further relates to addition or deletion of at least one amino acid in relation to any of the amino acid sequences according to the invention and may be an addition or deletion of from preferably 2 to 50 amino acids, such as from 10 to 20 amino acids, for example from 20 to 30 amino acids, such as from 40 to 50 amino acids.

However, additions or deletions of more than 50 amino acids, such as additions from 50 to 100 amino acids, addition of 100 to 150 amino acids, addition of 150-250 amino acids, are also comprised within the present invention. Even additions or deletions of 500-2000 amino acids are within the scope of the present invention. The deletion and/or the addition may—independently of one another—be a deletion and/or an addition within a sequence and/or at the end of a sequence.

The polypeptide fragments according to the present invention, including any functional equivalents thereof, may in one embodiment comprise less than 250 amino acid residues, such as less than 240 amino acid residues, for example less than 225 amino acid residues, such as less than 200 amino acid residues, for example less than 180 amino acid residues, such as less than 160 amino acid residues, for example less than 150 amino acid residues, such as less than 140 amino acid residues, for example less than 130 amino acid residues, such as less than 120 amino acid residues, for example less than 110 amino acid residues, such as less than 100 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues.

“Functional equivalency” as used in the present invention is according to one preferred embodiment established by means of reference to the corresponding functionality of a predetermined fragment of the sequence.

It is understood that the polypeptide fragments of the invention may possess one or more types of post-translational modifications when expressed on the cell surface. These modifications may comprise, but are not limited to, glycosylation, phosphorylation, acylation, cross-linking, proteolytic cleavage, linkage to an antibody molecule, a membrane molecule, or another ligand.

It is an object of presently preferred embodiments of the present invention that the polypeptide comprises the amino acid sequence shown in SEQ ID NO.: 2, SEQ ID NO.: 4 or SEQ ID NO.: 6.

It is an object of presently most preferred embodiments of the present invention that the polypeptide consists of the amino acid sequence shown in SEQ ID NO.: 2, SEQ ID NO.: 4 or SEQ ID NO.: 6.

### Nucleic Acid Molecules

The embodiments of the present invention thus relate to polypeptides of the PfEMP1 class or sub-sequences hereof as well as nucleic acid molecules encoding such polypeptides or sub-sequences, wherein said polypeptides and sub-sequences comprise structures that are involved directly or indirectly in the binding to endothelial receptors and/or recognised by plasma antibodies from young semi-immune children.

The PFD1235w/MAL7P1.1, PF11_{—}0008, and PF13_{—}0003 genes presented in SEQ ID NO.: 1, SEQ ID NO.: 3, and/or SEQ ID NO.: 5 are members of Group A var genes and, in their widest perspective, the embodiments of the invention thus relate to nucleic acid molecules, which are characteristic in that they do not belong to the var1, var2 gene subfamily as defined in Salanti et al. 2002 and Salanti et al. 2003 or Group B, Group C, and Group B/C, but does not exclude Group B/A var genes as defined in Lavstsen et al. 2003.

Furthermore, nucleic acid molecules, which are complementary to the nucleic acid molecules of the invention as described above as well as polypeptides encoded by these nucleic acid molecules are within the scope of the invention.

One embodiment of the present invention relates to a nucleic acid molecule comprising at least one nucleotide sequence selected from the group consisting of at least one of
a) SEQ 1N NO.: 1, SEQ ID NO.: 3 and SEQ ID NO.: 5 or a sequence complementary thereof; and
b) a nucleotide sequence having at least 80% sequence identity to a); and
c) sub-sequences of a) or b) with a minimum length of 30 nucleotides; and
d) sub-sequences of a) or b) which comprises at least one sequence encoding a B-cell epitope;

As understood by the skilled adressee, the sequence identity of the nucleic acid molecules of the present invention will be higher the smaller the fragments or sub-sequences in order to be unique, thus in some embodiments of the present invention, the fragments or sub-sequences are of 60-150 nucleic acid long and has a sequence identity of 80-100%, such as 60-140 nucleic acid long, 60-130 nucleic acid long, 60-120 nucleic acid long, 60-110 nucleic acid long, 75-150 nucleic acid long, 80-140 nucleic acid long, 90-135 nucleic acid long, 90-150 nucleic acid long, 120-130 or 125-150 nucleic acid long all with a sequence identity of 80-100%, such as 80% identical, 81% identical, 82% identical, 83% identical, 84% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical or even 100% identical.

In other embodiments of the present invention, the fragments or sub-sequences are of 151-2200 nucleic acid long and has a sequence identity of 70-100%, such as 151-2100 nucleic acid long, 200-1700 nucleic acid long, 300-1600 nucleic acid long, 400-1500 nucleic acid long, 500-1400 nucleic acid long, 1000-1250 nucleic acid long, 1300-1350 nucleic acid long, 1400-1500 nucleic acid long, 200-1650 or 450-2000 nucleic acid long all with a sequence identity of 70-100%, such as 70% identical, 71% identical, 72% identical, 73% identical, 74% identical, 75% identical, 80% identical, 85% identical, 88% identical, 89% identical, 90% identical, 91% identical, 92% identical, 93% identical, 94% identical, 95% identical, 96% identical, 97% identical, 98% identical, 99% identical or even 100% identical.

In yet other embodiments of the present invention, the fragments or sub-sequences are of more than 2201 nucleic acid long and has a sequence identity of 60%-100%, such as more 2201 nucleic acid long, more 2300 nucleic acid long, more than 2400 nucleic acid long, more than 3500 nucleic acid long, more than 4000 nucleic acid long, more than 5000 nucleic acid long, more than 5500 nucleic acid long, more than 6000 nucleic acid long, more than 7000 nucleic acids long or 8500 nucleic acid long all with a sequence identity of 60-100%, such as 60% identical, 61% identical, 62% identical, 63% identical, 64% identical, 65% identical, 70% identical, 75% identical, 77% identical, 80% identical, 85% identical, 88% identical, 92% identical, 93% identical, 94% identical, 95% identical, 96% identical, 97% identical, 98% identical, 99% identical or even 100% identical.

With the proviso that the Exon 2 of SEQ ID NO.: 1, 3 and/or 5 is excluded

With the proviso that the “Fragment 1” and/or “Fragment 2” and/or “Fragment 3” and/or “Fragment 4” and/or “Fragment 5” and/or “Fragment 6” and/or “Fragment 7” and/or “Fragment 8” and/or “Fragment 9” and/or “Fragment 10” and/or “Fragment 11” and/or “Fragment 12” and/or “Fragment 13” and/or “Fragment 14” and/or “Fragment 15” and/or “Fragment 16” and/or “Fragment 17” “Fragment 18” and/or “Fragment 19” and/or “Fragment 20” and/or “Fragment 21” and/or “Fragment 22” and/or “Fragment 23” and/or “Fragment 24” and/or “Fragment 25” and/or “Fragment 26” of SEQ ID NO.: 1, SEQ ID NO.: 3, and/or SEQ ID NO.: 5 is excluded.

With the proviso that the “Fragment 27” and/or “Fragment 28” and/or “Fragment 29” and/or “Fragment 30” and/or of SEQ ID NO.: 1 and/or SEQ ID NO.: 5 is excluded.

With the proviso that the “Fragment 31” and/or “Fragment 32” of SEQ ID NO.: 1 is excluded.

With the proviso that the nucleotide sequence of SEQ ID NO.: 1, SEQ ID NO.: 3 and/or SEQ ID NO.: 5 is excluded.

The cDNA sequence encoding SEQ ID NO. 2, SEQ ID NO.: 4, and/or SEQ ID NO.: 6 in the parasite line 3D7 is provided in the sequence listing as SEQ ID NO.: 1, SEQ ID NO.: 3 and/or SEQ ID NO.: 5.

Again, it is apparent for all the aspects of the invention that the nucleic acid molecules of the invention may be less than or equal to any length between 30-10662 (SEQ ID NO.: 1); 30-8985 (SEQ ID NO.: 3) or 30-10041 (SEQ ID NO.: 5) nucleotides, such as less than or equal to 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 110, 120, 130, 140, 150, 160, 170, 180, 190, 200, 225, 250, 275, 300, 350, 400, 450, 500, 600, 700, 800, 900, 1000, 1250, 1500, 1750, 2000, 2500, 3000, 3500, 4000, 4500, 5000, 6000, 7000, 8000, 8100, 8200, 8300, 8400, 8500, 8600, 8700, 8800, 8900, 8955, 8956, 8957, 8958, 8959, 8960, 8961, 8962, 8963, 8964, 8965, 8966, 8967, 8968, 8969, 8970, 8971, 8972, 8973, 8974, 8975, 8976, 8977, 8978, 8979, 8980, 8981, 8982, 8983, 8984, 8985 nucleotides in length for SEQ ID NO.: 3 and further 9000, 9100, 9200, 9300, 9400, 9500, 9600, 9700, 9800, 9900, 10000, 10011, 10021, 10031, 10041 nucleotides in length for SEQ ID NO.: 5, and furthermore 10050, 10100, 10200, 10300, 10400, 10500, 10600, 10650, 10651, 10652, 10653, 10654, 10655, 10656, 10657, 10658, 10659, or 10660 nucleotides in length for SEQ ID NO.: 1.

Still with respect to all aspects of the invention it may be preferred that the nucleic acid molecules of the invention may have a length of 30-40, 30-50, 30-60, 30-70, 30-80, 30-90, 30-100, 30-200, 30-400, 30-500, 30-1000, 30-1200, 30-1300, 30-1400, 30-1500, 30-1600, 30-1700, 30-1800, 30-1900, 30-2000, 30-2250, 30-2500, 30-2750, 30-3000, 30-3500, 30-4000, 30-4500, 30-5000, 30-6000, 30-7000, 30-8000, 30-9000 nucleotides.

In preferred embodiments of the invention, sub-sequences of the nucleic acid molecules of the invention have a minimum length of 30 nucleotides and in even more preferred embodiments these sub-sequences are at least 300 nucleotides long.

Sequence identity of the nucleic acid molecules

Preferred nucleic acid embodiments further include nucleic acids encoding fragments of the polypeptide of the invention that are involved in interaction with endothelial receptors. In addition, it is an object of preferred embodiments that sub-sequences of the nucleic acid molecule of the invention comprise nucleotides encoding one or more B-cell epitopes and/or one or more T-cell epitopes.

Some characteristic structures lie within the peptide sequence of SEQ ID NO.: 2, SEQ ID NO.: 4 and/or SEQ ID NO.: 6 and therefore also within the nucleotide sequence encoding this peptide sequence. Such structures comprise, but are not necessarily limited to, a DBL20 domain followed by a C2 domain. On the other hand, some common features have been identified for proteins encoded by Group B, Group C, and Group B/C var genes including, but not limited to the DBL2δ domain. These features are not found within the amino acid sequence of SEQ ID NO.: 2, SEQ ID NO.: 4 and/or SEQ ID NO.: 6.

Further embodiments comprise nucleic acid molecules that complement full-length of SEQ ID NO.: 1, SEQ ID NO.: 3 and/or SEQ ID NO.: 5 or sequences identical in part hereto as well as nucleotide sequences that complement fragments of full-length SEQ ID NO.: 1, SEQ ID NO.: 3 and/or SEQ ID NO.: 5, or sequences identical in part hereto.

Preferred complementary nucleic acid molecules of the invention comprise nucleic acid molecules that are complementary to fragments of SEQ ID NO.: 1, SEQ ID NO.: 3 and/or SEQ ID NO.: 5, which have a nucleotide sequence that encodes a polypeptide or parts of a polypeptide that are involved in interaction with endothelial receptors. Additionally, preferred complementary nucleic acid molecules of the invention are complementary to sequences encoding one or more B-cell epitopes and/or one or more T-cell epitopes.

As discussed for the polypeptide-based compounds of the invention it is also apparent that the nucleotide based embodiments may represent only part of the full-length sequence. In addition these nucleotide sequences may be present in combination with exogenous sequences. For all the aspects of the invention, it is thus apparent that the nucleic acids molecules of the invention may include sequences that have anywhere between 1-100% sequence identity to the full-length sequence of SEQ ID NO.: 1, SEQ ID NO.: 3 and/or SEQ ID NO.: 5, such as at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97% or, preferably, 100% sequence identity to SEQ ID NO.: 1, SEQ ID NO.: 3 and/or SEQ ID NO.: 5, or a fragment or sub-sequence thereof.

It is to be understood that the nucleotide sequence of SEQ ID NO.: 1, SEQ ID NO.: 3 and/or SEQ ID NO.: 5, when present within the genome of the intact Plasmodium falciparum parasites as well as the polypeptide sequence of SEQ ID NO.: 2, SEQ ID NO.: 4 and/or SEQ ID NO.: 6, when present in or on the surface of intact red blood cells infected with P. falciparum are excluded from the scope of the present invention. This applies to all embodiments of the invention described in the present application. Compounds of the invention may however comprise sub-sequences of SEQ ID NO.: 1, SEQ ID NO.: 3 and/or SEQ ID NO.: 5 and sub-sequences of SEQ ID NO.: 2, SEQ ID NO.: 4 and/or SEQ ID NO.: 6 isolated and/or purified from the Plasmodium parasites or infected RBC.

### Vectors

In addition, recombinant polypeptides comprising sub-sequences of the amino acid sequence of SEQ ID NO.: 2, SEQ ID NO.: 4 and/or SEQ ID NO.: 6 may be generated by use of the above-mentioned nucleic acid embodiments. These can be cloned into vectors by the use of cloning techniques known in the art. The sequence encoding the polypeptide of interest is thereby linked to a heterologous promoter sequence. It may be preferred to optimise the codon context and codon pairing for the particular expression system. With respect to the polypeptide embodiments of the invention the incorporation of a secretory leader sequence may also be of use. The vector can be an expression vector in any of the mammalian, yeast, amphibian, insect, parasite, or bacterial expression systems known in the art. It is therefore apparent that, with the exception of Plasmodium infected RBC, prokaryotic and eukaryotic cells hereunder mammalian cells and transformed cell lines as well as cells in animals possessing nucleotide and/or amino acid embodiments described herein, are within the scope of the present invention.

### Cells and Cell Lines

Propagation of such cells or cell lines may be performed with the intention of providing recombinant forms of one or more of the nucleic acid or polypeptide embodiments of the invention in amounts that are sufficient for further processing or purification. It is therefore within the scope of the present invention to provide preparations of compounds, which comprise polypeptides of the invention as well as nucleic acid molecules encoding these polypeptides. Preparations of such compounds may have a desired degree of purity referring to the relative amounts of the desired polypeptide and for instance whole cell proteins and unwanted variants of the desired polypeptide as defined above. The existence of a wide range of protein purification and concentration techniques is known to the skilled artisan. These techniques include gel electrophoresis, ion-exchange chromatography, affinity and immunoaffinity chromatography, ceramic hydroxyapatite chromatography, differential precipitation, molecular sieve chromatography, isoelectric focusing, gel filtration, and diafiltration.

For the various types of chromatography, the desired molecules are suspended in a buffer, which promotes adhesion of the molecules to the active surface of the resin and are then applied to the chromatography column. Removal of contaminants is performed by washing the resin in a buffer of intermediate ionic strength or pH. Elution of the desired molecules is performed by changing the ionic strength or pH of the buffer to values that will promote the dissociation of the molecules from the active surface of the resin used. In the case of immunoaffinity chromatography, the polypeptide may be purified by passage through a column containing a resin to which is bound antibodies which are specific for at least a portion of the polypeptide. Furthermore, His- or GST tags may be added to the polypeptides of the invention. Subsequently, the resulting fusion proteins can be purified by affinity chromatography on for instance glutathione sepharose 4B and HIS tag Metal Chelate Affinity Chromatography.

It is readily apparent that a person skilled in the art can create nucleic acid molecules of virtually any length by ligating a nucleic acid molecule encoding any of the amino acid sequences of the present invention or any part thereof to an exogenous nucleotide sequence. Recombinant nucleic acid molecules generated by this approach are embodiments of the invention. A recombinant construct can be capable of replicating autonomously within a host cell or, alternatively, it can become integrated into the chromosomal DNA. Such a recombinant nucleic acid molecule can comprise a sequence of genomic DNA, cDNA, synthetic or semi-synthetic origin. Again, it is preferred that such nucleic acid molecules are encoding one or more B-cell epitopes and/or one or more T-cell epitopes.

The nucleic acid embodiments of the present invention can be altered by genetic engineering so as to introduce substitutions, deletions and/or additions. In preferred embodiments of the invention, these alterations will provide for sequences encoding functionally equivalent molecules or molecules with the same or improved properties. Such changes of the polypeptide embodiments can be generated using techniques that are known to a person skilled in the art, including random mutagenesis and site-directed mutagenesis.

The use of recombinant polypeptides of the invention may be preferred when it is required that the preparations of these polypeptides are essentially free of any other antigen with which they are natively associated, i.e. free of any other antigen from Plasmodium parasites. As an alternative this may also be accomplished by synthesizing the polypeptide fragments by the well-known methods of solid or liquid phase peptide synthesis.

### Pharmaceutical Compositions

Additional aspects of the present invention relate to pharmaceutical compositions based on any of the polypeptide embodiments of the invention. In a presently preferred embodiment these pharmaceutical compositions relates to compositions capable of eliciting an immune response, which may or may not be in form of a vaccine. Preferably, such a composition comprises at least one amino acid sequence according to the present invention.

It is an object of presently preferred embodiments of the present invention to provide such an amino acid sequence selected from the group consisting of at least one of
a) SEQ ID NO.: 2, SEQ ID NO.: 4 and SEQ ID NO.: 6 and
b) a sequence having at least 80% sequence identity to a); and
c) sub-sequences of a) or b) with a minimum length of 30 amino acids; and
d) sub-sequences of a) or b) comprising at least one B-cell epitope.

Alternatively, the pharmaceutical composition according to the present invention may be based on any of the nucleotide embodiments of the invention. In a preferred embodiment, the pharmaceutical composition comprises at least one nucleotide sequence selected from the group consisting of at least one of
a) SEQ ID NO.: 1, SEQ ID NO.: 3 and SEQ ID NO.: 5 or a sequence complementary thereof; and
b) a nucleotide sequence having at least 80% sequence identity to a); and
c) sub-sequences of a) or b) with a minimum length of 30 nucleotides; and
d) sub-sequences of a) and b) which comprise at least one sequence encoding a B-cell epitope.

Such compositions may or may not further comprise a vector containing said amino acid or nucleotide sequence.

In a specially preferred embodiment, any of the pharmaceutical compositions described in the present application may further comprise a pharmaceutically acceptable carrier and/or an adjuvant.

Peptides, proteins and carbohydrates antigens are usually poorly immunogenic, or not immunogenic at all, when administered on their own. Better delivery systems and adjuvants may be needed to improve the ease of delivery and immunogenicity. In particular, in vivo immunisation with peptides and proteins to generate MHC class I specific response has been difficult. One possible technique to solve this is the use of ISCOMs, which are lipid carriers that act as adjuvants but have minimal toxicity, and they appear to load peptide and proteins into the cell cytoplasma, allowing MHC class I restricted T-cell response to these peptides, which make them useful in human immunisation.

Pharmaceutical compositions comprising the nucleotide and polypeptide embodiments of the invention can be produced by conventional techniques so that the said sequences are present as monomeric, multimeric or multimerised agents. Furthermore, antibodies generated from the polypeptide embodiments of the invention may constitute part of such pharmaceutical compositions. In addition to the active ingredients, pharmaceutical compositions may further comprise one or more physiologically acceptable carriers, proteins, supports, adjuvants as well as components that may facilitate the delivery of the active components of the compositions. As described above, a large number of adjuvants are available including but not limited to Freund's adjuvant, mineral gels such as aluminium hydroxide, and surface-active substances such as lysolecithin, pluronic polyols, polyanions, peptides, oil emulsions, keyhole limpet hemocyanin, and dinitrophenol. As a pharmaceutical composition, the nucleic acid and peptide embodiments of the invention will be purified and processed through one or more formulation steps. A large variety of formulation buffers will be physiologically acceptable, such as phosphate, citrate, and other organic acids.

It is further understood that a pharmaceutical composition must be clinically safe. More specifically, it must be free of virus and bacteria that can cause infection upon administration of the composition to a subject. It may therefore be necessary to process the composition through on or more steps of virus filtration and/or inactivation. The removal of virus by filtration can be obtained by passing the composition through a nanofilter, whereas virus inactivation can be accomplished by the addition of various detergents and/or solvents or other antiviral compounds to the composition.

### Antibodies

The polypeptide and/or nucleic acid embodiments of the invention may be used in their purified form to generate various types of antibodies, and it is understood that such antibodies will also be considered as compounds of the invention. These antibodies may include, but are not limited to polyclonal, monoclonal, chimeric, single chain, Fab fragments and fragments produced by a Fab expression library. A person skilled in the art knows that antibodies can be produced by e.g. immunisation of various hosts including goats, rabbits, rats, and mice. The term ‘immunisation’ refers to the injection of a polypeptide with immunogenic properties. Depending on the host species various types of adjuvants can be used in order to increase the immunological response including but not limited to Freund's adjuvant, mineral gels such as aluminium hydroxide, and surface-active substances such as lysolecithin, pluronic polyols, polyanions, peptides, oil emulsions, keyhole limpet hemocyanin, and dinitrophenol.

It is preferred to use shorter sequences of the polypeptide of the invention fused to a powerful immunogenic molecule such as keyhole limpet hemocyanin resulting in the production of antibodies against this chimeric molecule. Accordingly, antibodies capable of recognising SEQ ID NO. 2, SEQ ID NO.: 4 and/or SEQ ID NO.: 6 can be produced by injection of synthetic peptides consisting of as little as 3 to 30 amino. Thus, it is an object of preferred embodiments of the present invention to provide such small synthetic peptides of 3-250 amino acids, such as but not limited to 15-150 amino acids, 20-140 amino acids, 30-130 amino acids, 40-120 amino acids, 50-110 amino acids, 60-100 amino acids, 70-90 amino acids, 80-85 amino acids corresponding to a particular sequence of the SEQ ID NO. 2, SEQ ID NO.: 4 and/or SEQ ID NO.: 6 polypeptides. As an alternative, a more diverse set of antibodies can be generated by injection of a purified polypeptide embodiment of the invention.

Monoclonal antibodies directed against any of the polypeptides of the present invention, such as a purified polypeptide embodiment of the invention, can be produced using any of the conventional techniques that provide for the production of antibodies from cell lines in continuous culture. These techniques include the hybridoma technique, the human B-cell hybridoma technique, and the EBV-hybridoma technique.

It will be readily appreciated that polypeptides of the invention can be incorporated into vaccines capable of inducing protective immunity against a specific subtype of malaria. In relation to the present invention it is preferred that the vaccine is directed specifically against the infectious activity of Plasmodium falciparum in the brain or adhesion to endothelial cells, which is characteristic of severe malaria.

One important aspect of the present invention therefore relates to a vaccine comprising one or more B-cell epitopes from a polypeptide encoded by any of the nucleotides according to the present invention. This vaccine is characterised in that it induces an antibody response wherein said antibody specifically recognises a molecule expressed on the surface of an intact erythrocyte infected by VSA_{SM} type parasites or parasites that have been selected for their ability to mediate adhesion to endothelial receptors. Generally, this molecule is recognised by the antibodies from semi-immune children or children suffering from severe malaria.

In preferred embodiments, antibodies directed against the polypeptides and/or nucleic acids of the invention can be administered to a subject in order to provide protection against the retention and sequestration of iRBC to endothelial cells which is characteristic of severe malaria.

Effective amounts of an agent that will promote an immune response against a compound of the present invention can be administered to subjects living in endemic areas so as to prevent the contraction of malaria.

In another embodiment, a subject believed to be at risk for contracting malaria may be identified either by conventional methods or by one of the in vitro diagnostic techniques, which constitute other embodiments of the present invention. An effective amount of an agent that inhibits SEQ ID NO.: 2, SEQ ID NO.: 4, SEQ ID NO.: 6 and/or homologues hereof mediated sequestration or elicits an immune response in a subject can then be administered to this subject.

Another embodiment of the present invention relates to an isolated antibody or isolated antiserum induced in response to one or more polypeptides according to the invention and/or to one or more nucleic acids as defined in the present application.

Another embodiment of the present invention relates to an isolated antibody or isolated antiserum induced in response to one or more polypeptides according to the invention and/or to one or more nucleic acids as defined in the present application, wherein said antibody is capable of binding to a molecule expressed on the surface of an intact erythrocyte infected by a parasite causing malaria.

Another embodiment of the present invention relates to an isolated antibody or isolated antiserum induced in response to one or more polypeptides according to the invention and/or to one or more nucleic acids as defined in the present application, wherein said antibody is capable of recognising paracites selected in vitro for expression of VSA_{SM}.

A presently preferred embodiment relates to an antibody as described by the present application, wherein said antibody is capable of binding to a molecule expressed on the surface of an intact erythrocyte infected by a parasite capable of mediating cyto-adhesion of intact erythrocyte infected by a parasite to human endothelial cells, but not the CD36 receptor.

### Vaccines

Additional aspects of the present invention relate to vaccines based on any of the embodiments of the invention, such as but not limited to a vaccine comprising any of the polypeptides, the nucleic acids or the recombinant vectors according to the present invention, said vaccine characterised in that it induces an immune response, wherein said immune response specifically recognises a molecule expressed on the surface of an intact erythrocyte infected by a parasites.

In one embodiment such a vaccine relates to a vaccine comprising one or more B-cell and/or T-cell epitopes originating from any of the polypeptides, the nucleic acids or the recombinant vectors of the present invention, said vaccine characterised in that it induces an immune response, wherein said immune response specifically recognises a molecule expressed on the surface of an intact erythrocyte infected by a parasites.

One presently preferred embodiment of the present invention relates to a polypeptide-based vaccine comprises at least one amino acid sequence selected from the group consisting of at least one of
a) SEQ ID NO.: 2, SEQ ID NO.: 4 and/or SEQ ID NO.: 6 and
b) a sequence having at least 80% sequence identity to a); and
c) sub-sequences of a) or b) with a minimum length of 30 amino acids; and d) sub-sequences of a) or b) comprising at least one B-cell epitope.

Sub-sequences of the polypeptide of the invention used in a vaccine may be any of the above mentioned amino acid lengths and in addition to these fragments or sub-sequences of the polypeptide of the invention, larger polypeptides comprising sub-sequences of the invention as part of their sequence, are also embodiments of the present invention. It is preferred, however, that these sub-sequences have a minimum length of 30 amino acids and that they are at least 80% identical to a region of comparable length within the sequence of SEQ ID NO.: 2, SEQ ID NO.: 4 and/or SEQ ID NO.: 6.

In recent years there has been increased focus on nucleic acid vaccines. Other aspects of the present invention therefore concern genetic immunisation in which nucleotide based vaccines such as vaccines based on DNA molecules or on RNA molecules, which result in the expression of one or more B-cell and/or T-cell epitopes from a polypeptide encoded by a member of the SEQ ID NO.: 1, 3, and/or 5 gene family. As for the polypeptide based vaccine this vaccine is characterised in that it induces an antibody response wherein said antibody specifically recognises a molecule expressed on the surface of an intact erythrocyte infected by VSA_{SM} type parasites that have been selected for their ability to mediate adhesion to endothelial cells or for increased antibody recognition by plasma from young semi-immune children.

One embodiment of the present invention relates to a nucleotide based vaccine, such as a DNA vaccine, which results in the expression of a polypeptide comprising one or more B-cell and/or T cell epitopes from any of the polypeptide sequences of the present invention, wherein said vaccine is capable of inducing an immune responds which specifically recognises a molecule expressed on the surface of an intact erythrocyte infected by parasites, and wherein said parasites furthermore has a VSA_{SM} phenotype that have been selected for antibody recognition or adhesion to endothelial receptors in vitro, and wherein said molecule is recognised by antibodies from young children living in areas of high malaria transmission intensity.

One embodiment relates to a DNA vaccine comprising at least one nucleic acid sequences to the present invention, wherein said vaccine is capable of inducing an immune response, wherein said immune response specifically recognises a molecule expressed on the surface of an intact erythrocyte infected by parasites.

Another embodiment relates to a natural, synthetic or recombinant DNA or RNA vaccine having any nucleotide sequence according to the present invention, wherein the vaccine is capable of eliciting development of anti-Plasmodium antibodies.

In a preferred embodiment, the present invention relates to a nucleotide-based vaccine, which may be a DNA or RNA vaccine, comprising a vector comprising at least one nucleotide sequence selected from the group consisting of at least one of
a) SEQ ID NO.: 1, SEQ ID NO.: 3 and SEQ ID NO.: 5 or a sequence complementary thereof; and
b) a nucleotide sequence having at least 80% sequence identity to a); and
c) sub-sequences of a) or b) with a minimum length of 30 nucleotides; and
d) sub-sequences of a) and b) which comprise at least one sequence encoding a B-cell epitope.

The vaccine may thus comprise any of the sub-sequences of the nucleotide sequence of the invention, which may have any of the sequence identities described above. It is preferred, however, that these sub-sequences have a minimum length of 30 nucleotides and that they are at least 80% identical to a region of comparable length within the sequence of SEQ ID NO.: 1, SEQ ID NO.: 3 and/or SEQ ID NO.: 5.

According to this aspect of the invention one approach is to incorporate the DNA encoding a polypeptide of the invention or parts hereof into a viral or bacterial vector. The following organisms, among numerous others, may be employed for this purpose: Coxsackie virus, vaccinia virus, Salmonella typhi or Salmonella typhimurium (for oral administration). In each case the carrier organism must be acquired by the host cell and the relevant DNA sequences used for production of the polypeptide of the invention or parts hereof. These in turn are recognised as abnormal by the host or recipient and an immune response ensues.

Alternatively, the parasite nucleic acid sequence may be incorporated into an RNA virus or used to prepare viral replicons. This approach allows for the delivery of coding sequences, such as mRNA, to the host cell without risking a replicative, infectious process.

In order to obtain expression of immunogenic polypeptides it is required that elements of a nucleotide-based vaccine are capable of entering into the relevant target cells of the subject receiving such a vaccine. Therefore, preferred embodiments of the invention include vaccines, which further comprise one or more agents and/or vectors to facilitate such entry.

In a further preferred embodiment, the vector component of a nucleotide-based vaccine comprises a promoter for driving the expression in a mammalian cell line, a nucleotide sequence encoding a leader peptide for facilitating secretion/release of a polypeptide sequence from a mammalian cell, and a terminator.

The simple concept of a nucleotide-based vaccine is the inoculation of a recipient using the relevant DNA sequence alone. This ‘naked DNA’ approach avoids the administration of polypeptide directly, but its effectiveness depends on the ability of the host cell to utilise the injected DNA as a template for RNA and subsequent protein synthesis.

It is anticipated that the principal value of providing SM-specific protective immunity to sporozoite-induced infection will be, but not limited to individuals who have not previously been exposed to malaria or have only had a few cases of malaria. In the case of severe malaria such individuals will be infants and young children in endemic areas, travellers/tourists, soldiers or individuals from a non-malaria endemic area moving to or travelling in endemic areas.

While not being limited by way of theory it is believed that the protection against malaria obtained by the use of a vaccine is most likely a result of IgGs blocking the interaction between the iRBC and endothelial receptors in various organs of the body such as, but not limited to the brain. It is also possible, however, that opsonized erythrocytes are killed by macrophages or T-cells, either by fagocytosis or by other means.

In a preferred embodiment, the vaccine is therefore capable of inducing an IgG response and, accordingly, it comprises a polypeptide comprising one or more B-cell epitopes. It is desirable, however, that polypeptides comprising one or more T-cell epitopes are also part of the vaccine since assistance from T-cells may be required in order to obtain a good antibody response.

In another preferred embodiment of the invention, the vaccine is therefore based on the use of polypeptides of the invention wherein said polypeptides comprises one or more B-cell epitopes in combination with one or more T-cell epitopes. In a another preferred embodiment of the invention, the vaccine comprises B-cell epitopes in combination with T-cell epitopes originating from an exogenous molecule, and in an further preferred embodiment, the peptides of the vaccine comprises only B-cell epitopes. In equally preferred embodiments of the invention, the vaccine is based on nucleotide sequences encoding polypeptides, which have the characteristics with respect to antigen epitopes described above.

Techniques exist for enhancing the antigenicity of immunogenic peptides including incorporation of these into a multimeric structure, binding to a highly immunogenic protein carrier, for example, keyhole limpet hemocyanin, or diptheria toxoid, and administration in combination with adjuvants or any other enhancers of immune response. Furthermore, it will be understood that polypeptides specific for a plurality of Plasmodium stages and species may be incorporated in the same vaccine composition to provide a multivalent vaccine. In addition, the vaccine composition may comprise antigens to provide immunity against other diseases in addition to malaria.

Immunogenic polypeptides of the invention as well as nucleic acid molecules encoding such polypeptides may be injected as is, or for convenience of administration, it can be added to pharmaceutically acceptable carriers or diluents. Suitable pharmaceutically acceptable carriers will be apparent to those skilled in the art, and include water and other polar substances, including lower molecular weight alkanols, polyalkanols such as ethylene glycol, polyethylene glycol, and propylene glycol as well as non-polar carriers.

Routes of administration, antigen dose, number and frequency of injections are all matters of optimisation within the scope of ordinary skill in the art, particularly in view of the fact that there is already experience in the art of providing protective immunity by the injection of irradiated sporozoites. Protective antibodies are usually best elicited by a series of 2 to 3 doses given about 2 to 3 weeks apart. The series can be repeated when concentrations of circulating antibodies in the vaccinee drops. The polypeptide is present in the vaccine in an amount sufficient to induce an immune response against the antigenic polypeptide and thus to protect against Plasmodium infection thereby protecting the subject against malaria.

Vaccination protocols can include the identification of a subject in need of a vaccine, for instance infants and young children living in regions populated with P. falciparum or non-immune individuals e.g. tourist/travellers and soldiers travelling through such regions, and administration of one or more effective doses of the vaccine to this subject.

In some aspects, the present invention can be used to both inhibit the adhesion of iRBC to endothelial receptors and to generate an immune response directed at SEQ ID NO. 2, SEQ ID NO.: 4 and/or SEQ ID NO.: 6. It is therefore within the scope of the invention to provide uses of any of the polypeptides of the present invention as medicaments that are therapeutically or prophylactically useful or both.

Alternatively, such therapeutic and prophylactic effects can be obtained as a result of the expression of polypeptides of the invention within a diseased subject or a subject at risk for contracting malaria. Therefore, it is also within the scope of the invention to provide uses of any of the nucleic acid molecules of the present invention as medicaments that are therapeutically or prophylactically useful or both.

A currently particular preferred embodiment of the present invention relates to a vaccine comprising at least one nucleic acid according to the present invention or at least one vector according to the present invention, the vaccine effecting in vivo expression of at least one antigen by a subject, to whom the vaccine has been administered, the amount of expressed antigen being effective to confer substantially increased resistance to malaria caused by Plasmodium falciparum.

Embodied in the invention is also a method for generating a vaccine against malaria comprising
a) injecting a sequence according to any of claims 1-14 and/or 15-28 in a subject
b) enabling said subject to generate antibodies specifically recognising any of the polypeptide sequences according to claim 1-14
c) purify said antibodies
d) selecting antibodies having cross-reactivity to parasites causing malaria
e) selecting antibodies having the ability to inhibit adhesion to endothelial cells.

### Medical Use

### Polypeptides

One presently particular preferred aspect of the present invention relates to an isolated polypeptide comprising an amino acid sequence selected from the group consisting of at least one of SEQ ID NO.: 2, SEQ ID NO.: 4 and SEQ ID NO.: 6 for use as a medicament.

In a presently preferred embodiment the present invention relates to use of any of the amino acid sequences according to the present invention capable of mediating cyto-adhesion of intact erythrocyte infected by a parasite to human endothelial cells, but not to the CD36 receptor.

Such amino acid sequences can be segregated by an assay known to the skilled person comprising Chinese hamster ovary (CHO) cells transfected to express human CD36 and cultured by standard methodology to measure iRBC adhesion to these receptors. In brief, parasites can be radiolabelled by incubating the cultures overnight in the presence of ³H-phenylalanin (1 MBq for a standard culture containing 200 μl packed RBC). Wildtype and CD36-transfected CHO cells can be grown to a monolayer in 96-well microtitre plates (Nunc, Roskilde, Denmark). Late-stage-enriched iRBC (100 μl, 1×10⁷ RBC/ml) can be added to the CHO cell monolayer and incubated for one hour at 37° C. before unbound iRBC can be washed away from the CHO cell monolayer. Finally, the number of CHO-adhering iRBC could be determined by liquid scintillation spectrometry.

In another presently preferred embodiment the present invention relates to use of any of the amino acid sequences according to the present invention, wherein said amino acid is consistently up-regulated after antibody selection-induced change from VSA_{UM} to VSA_{SM} expression or following selection for adhesion to bone marrow derived endothelial cells. Said amino acids do not belong to PfEMP1 groups B, C, B/C, var1 or var2 as defined by Lavstsen et al. 2003, and are further characterised lacking 1-2 cysteine residues in DBLα homology group G (Smith et al. 2000) compared to most PfEMP1 molecules in groups B and C.

For the present aspects of the invention it applies, as describe by the inventors, that it is an object of preferred embodiments of the present invention to provide any polypeptides, which are subject to antibody recognition by antibodies in sera from young children living in areas of high malaria transmission intensity and/or capable of mediating cyto-adhesion of intact erythrocyte infected by a parasite to endothelial cells, but not the CD36 receptor, for use as a medicament. Thus in one embodiment, the amino acid sequence may have at least 80% sequence identity to SEQ ID NO.: 2, SEQ ID NO.: 4 and SEQ ID NO.: 6.

It should be understood by the skilled addressee that any feature and/or aspect discussed above in connection with the sequence identity of the polypeptides according to the invention apply by analogy to antibodies according to the invention.

In another embodiment, the present invention relates to medical uses of an isolated polypeptide, wherein the amino acid sequence is a sub-sequence of with a minimum length of 10 amino acids.

Again, it should be understood that any length of an isolated polypeptide described above by analogy applies to the present aspect of the invention.

A presently preferred embodiment relates to medical use of an isolated polypeptide comprising the amino acid sequence shown in SEQ ID NO:2.

Another presently preferred embodiment relates to medical use of an isolated polypeptide comprising the amino acid sequence shown in SEQ ID NO:4.

Another presently preferred embodiment relates to medical use of an isolated polypeptide comprising the amino acid sequence shown in SEQ ID NO:6.

A particular presently preferred embodiment relates to medical use of an isolated polypeptide consisting of the amino acid sequence shown in SEQ ID NO:2.

Another particular presently preferred embodiment relates to medical use of an isolated polypeptide consisting of the amino acid sequence shown in SEQ ID NO:4.

Another particular presently preferred embodiment relates to medical use of an isolated polypeptide consisting of the amino acid sequence shown in SEQ ID NO:6.

One embodiment of the present invention relates to medial use of an isolated polypeptide according to the present invention, wherein the amino acid sequence has at least 80% sequence identity to SEQ ID NO:2.

Another embodiment of the present invention relates to medial use of an isolated polypeptide according to the present invention, wherein the amino acid sequence has at least 80% sequence identity to SEQ ID NO:4.

Yet another embodiment of the present invention relates to medial use of an isolated polypeptide according to the present invention wherein the amino acid sequence has at least 80% sequence identity to SEQ ID NO:6.

One specially preferred embodiment of the present invention relates to medical use of any of the polypeptides described herein, wherein the amino acid sequence is capable of mediating cyto-adhesion of intact erythrocyte infected by a parasite to human endothelial cells, but not to the CD36 receptor.

Another specially preferred embodiment of the present invention relates to medical use of any of the polypeptides described herein wherein the amino acid is consistently up-regulated after antibody selection-induced change from VSA_{UM} to VSA_{SM} expression.

### Nucleic Acids

The embodiments of the present invention thus relate to polypeptides of the PfEMP1 class or sub-sequences hereof as well as nucleic acid molecules encoding such polypeptides or sub-sequences, thus one aspect of the present invention relates to medical uses of an isolated nucleic acid comprising a nucleotide sequence selected from the group consisting of at least one of SEQ ID NO.: 1, SEQ ID NO.: 3 and SEQ ID NO.: 5 for use as a medicament.

As described by the present inventors the aim of the present invention is to provide a nucleic acid sequence encoding a polypeptide which is capable of mediating cyto-adhesion of intact erythrocyte infected by a parasite to human endothelial cells and/or provide a nucleic acid sequence which is consistently upregulated after antibody selection-induced change from VSA_{UM} to VSA_{SM} expression.

This one embodiment relates to medical use of a nucleic acid, wherein the nucleotide sequence has at least 80% sequence identity to SEQ ID NO.: 1, SEQ ID NO.: 3 and/or SEQ ID NO.: 5.

Another presently preferred embodiment relates to medical use of a nucleic acid wherein the nucleotide sequence is a sub-sequence of with a minimum length of 30 nucleotides.

All the features described herein relating to the nucleic acid molecules of the present invention are also applicable as embodiments relating to the medical use of said nucleic acids, and vice versa.

Another presently preferred embodiment relates to medical use of a nucleic acid, wherein the nucleic acid comprises the nucleotide sequence shown in SEQ ID NO:1.

Another presently preferred embodiment relates to medical use of a nucleic acid, wherein the nucleic acid consists of the nucleotide sequence shown in SEQ ID NO:1.

Another presently preferred embodiment relates to medical use of a nucleic acid, wherein the nucleic acid comprises the nucleotide sequence shown in SEQ ID NO:3.

Another presently preferred embodiment relates to medical use of a nucleic acid, wherein the nucleic acid consists of the nucleotide sequence shown in SEQ ID NO:3.

Another presently preferred embodiment relates to medical use of a nucleic acid, wherein the nucleic acid comprises the nucleotide sequence shown in SEQ ID NO:5.

Another presently preferred embodiment relates to medical use of a nucleic acid, wherein the nucleic acid consists of the nucleotide sequence shown in SEQ ID NO:5.

Another presently preferred embodiment relates to medical use of a nucleic acid, wherein the nucleotide sequence has at least 80% sequence identity to SEQ ID NO:1.

Another presently preferred embodiment relates to medical use of a nucleic acid, wherein the nucleotide sequence has at least 80% sequence identity to SEQ ID NO:3.

Another presently preferred embodiment relates to medical use of a nucleic acid, wherein the nucleotide sequence has at least 80% sequence identity to SEQ ID NO:5.

Another presently preferred embodiment relates to medical use of a nucleic acid, wherein the sequence is consistently upregulated after antibody selection-induced change from VSA_{UM} to VSA_{SM} expression.

Another presently preferred embodiment relates to medical use of a nucleic acid, wherein the nucleic acid sequence encodes a polypeptide which is capable of mediating cyto-adhesion of intact erythrocyte infected by a parasite to human endothelial cells, but not the CD36 receptor.

Another presently preferred embodiment relates to medical use of a nucleic acid, wherein the nucleic acid sequence encodes a polypeptide which is recognised by plasma from young semi-immune children.

In a further preferred embodiment nucleic acid sequence is a re-codonised sequence. Particularly preferred are sequences that are recodonised in order to enhance or optimise expression of the resulting protein or polypeptide in a given expression system.

### Vector

Another aspect of the present invention relates to a recombinant vector comprising any of the nucleic acids defined in the present application operably linked to one or more control sequences for use as a medicament.

### Further Medical Uses

In another aspect, the present invention relates to use of any of the polypeptides according to the present invention for the manufacture of a composition to be administered in order to prophylactically or therapeutically reduce the incidence, prevalence or severity of malaria in a subject.

Thus, in one embodiment, the present invention relates to use of any of the polypeptides according to the present invention for the manufacture of a vaccine for prophylac of malaria.

In another embodiment the present invention relates to use of any of the polypeptides according to the present invention for the manufacture of a composition for vaccination against malaria.

As will be apparent, the present invention further relates to use of any nucleic acid according to the present invention for the manufacture of an composition to be administered in order to prophylactically or therapeutically reduce the incidence, prevalence or severity of malaria in a subject.

In one embodiment, the present invention relates to use of any nucleic acid according to the present invention for the manufacture of a vaccine for malaria prophylaxis.

In another embodiment the present invention relates to use of any nucleic acid according to the present invention for the manufacture of a composition for vaccination against malaria.

As the skilled adressee would recognise the present invention also relates to use of a recombinant vector according to the present invention for the manufacture of a composition to be administered in order to prophylactically or therapeutically reduce the incidence, prevalence or severity of malaria in a subject.

In one embodiment the present invention relates to use of a recombinant vector according to the present invention for the manufacture of a vaccine for prophylactic treatment of malaria.

In another embodiment the present invention relates to use of a recombinant vector according to the present invention for the manufacture of a composition for vaccination against malaria.

In a presently preferred embodiment of the present invention said malaria is caused by Plasmodium falciparum.

### Treatment

It should be understood that any feature and/or aspect discussed above in connection with the uses according to the invention apply by analogy to methods of treatment according to the invention.

Thus, is one aspect the present invention relates to a method for prophylactically or therapeutically reduce the incidence, prevalence or severity of malaria in an subject said method comprising administering to said subject an effective amount of a polypeptide, a nucleic acid or a recombinant vector according to the present invention.

In one embodiment the present invention relates to a method for the prophylactic treatment of malaria in a subject, said method comprises administering to said subject an effective amount of a polypeptide, a nucleic or a recombinant vector according to the present invention.

In another embodiment the present invention relates to a vaccination method against malaria in a subject, said vaccination method comprising administering to said subject an effective amount of a polypeptide, a nucleic acid or a recombinant vector according to the present invention.

### Composition

Another aspect of the present invention relates to use of the polypeptides and/or the nucleic acids as defined by the present application for the preparations of a composition. Thus in one embodiment the invention relates to a composition comprising a polypeptide according to the invention or a nucleic acid according to the invention and a pharmaceutically acceptable diluent, carrier or adjuvant.

In a presently preferred embodiment the composition is an immunogenic composition.

In a presently most preferred embodiment the composition induces an IgG/IgM antibody response.

### Production of Pharmaceuticals

Another aspect of the present invention may be the production of pharmaceuticals based on polypeptides of the invention or sub-sequences hereof or nucleic acid sequences encoding such molecules, as described above. Such pharmaceuticals may also include agents such as but not limited to other polypeptides and in particular antibodies, which are capable of modulating and/or inhibit the adhesion of SEQ ID NO.: 2, SEQ ID NO.:4, SEQ ID NO.:6 and/or homologous hereof to endothelial receptors.

Accordingly, it is within the scope of the invention to provide the use of any of the amino acid sequences according to the present invention for the manufacture of a composition, such as an immunogenic composition, which is to be administered in order to prophylactically or therapeutically reduce the incidence, prevalence or severity of severe malaria (SM) in an individual non-immune to SM causing parasites.

In addition, the invention also relates to the use of a nucleic acid molecule according to the present invention for the manufacture of a composition, such as an immunogenic composition, which is to be administered in order to prophylactically or therapeutically reduce the incidence, prevalence or severity of severe malaria in an individual non-immune to SM causing parasites.

Delivery of these pharmaceuticals can be performed by any conventional route including, but not limited to, transdermal, parenteral, gastrointestinal, transbronchial, and transalveolar administration.

### Biotechnological Tools

The use of the nucleic acid and polypeptide-based embodiments of the present invention can also extend to their use as biotechnological tools and as components of diagnostic assays. Thus, one embodiment relates to an in vitro diagnostic method, said method comprising contacting a sample with any of the polypeptides according to the present invention under conditions allowing an in vitro immunological reaction to occur between said polypeptide and the antibodies possibly present in said sample, and in vitro detect the antigen-antibody complexes possibly formed.

In a presently preferred embodiment said diagnostic assay, further relates to an in vitro diagnostic method, wherein a disease-state profile for a tested subject is generated by determining the concentration or expression level in a sample of any of the polypeptide and/or nucleic acid sequences as defined in the present application.

Additional embodiments of the invention therefore include an in vitro diagnostic method, which comprises contacting a sample such as a tissue or biological fluid with a polypeptide comprising a sequence selected from the group consisting of at least one of
a) SEQ ID NO.: 2, SEQ ID NO.: 4, and SEQ ID NO.: 6; and
b) a sequence having at least 80% sequence identity to a); and
c) sub-sequences of a) or b) with a minimum length of 30 amino acids; and
d) sub-sequences of a) or b) comprising at least one B-cell epitope
   under conditions allowing an in vitro immunological reaction to occur between said polypeptide composition and the antibodies possibly present in the biological sample, and the in vitro detection of the antigen-antibody complexes possibly formed. In one preferred embodiment the polypeptide is immobilised on a solid support.

Other embodiments include an in vitro diagnostic method, which comprises contacting a sample such as a tissue or biological fluid with a nucleotide composition comprising a sequence selected from the group consisting of at least one of
a) SEQ ID NO.: 1, SEQ ID NO.: 3, and SEQ ID NO.: 5 or a sequence complementary thereof; and
b) a nucleotide sequence having at least 80% sequence identity to a); and
c) sub-sequences of a) or b) with a minimum length of 18 nucleotides under conditions allowing an in vitro reaction to occur between said nucleotide composition and e.g. primers and probes.

In some aspects, the nucleic acid embodiments are employed as nucleic acid probes in hybridisation assays, in cloning, or as primers for polymerase chain reaction (PCR). Similarly, the polypeptide-based embodiments can be used as components of immunological reactions such as ELISA, radio-immunoassays (RIA) and adhesion-blocking assays. The scope of such work can be, for example, to characterise SEQ ID NO.: 2, SEQ ID NO.: 4, and/or SEQ ID NO.: 6 or regions of SEQ ID NO.: 2, SEQ ID NO.: 4, and/or SEQ ID NO.: 6 involved in interaction with endothelial receptors or antibodies

In some diagnostic embodiments, nucleic acids complementary to the nucleic acid molecules of the invention or fragments hereof are used to identify SEQ ID NO.: 1, SEQ ID NO.: 3, and/or SEQ ID NO.: 5 nucleic acids (e.g. mRNA) present in a biological sample, for instance a tissue sample or a sample of body fluid such as blood or serum. In a preferred diagnostic embodiment, nucleic acid molecules complementary to fragments of SEQ ID NO.: 1, SEQ ID NO.: 3, and/or SEQ ID NO.: 5 comprising sequences, which are not found in nucleic acids encoding other SEQ ID NO.: 2, SEQ ID NO.: 4, and/or SEQ ID NO.: 6 proteins, are used to identify SEQ ID NO.: 1, SEQ ID NO.: 3, and/or SEQ ID NO.: 5 nucleic acids (e.g. mRNA) present in a biological sample.

In other diagnostic embodiments, nucleic acids identical to the nucleic acid molecules of the invention or fragments hereof are used to identify SEQ ID NO.: 1, SEQ ID NO.: 3, and/or SEQ ID NO.: 5 nucleic acids (e.g. mRNA) present in a biological sample, for instance a tissue sample or a sample of body fluid such as blood or serum. In a preferred diagnostic embodiment, nucleic acid molecules identical to fragments of SEQ ID NO.: 1, SEQ ID NO.: 3, and/or SEQ ID NO.: 5 comprising sequences, which are not found in nucleic acids encoding other SEQ ID NO.: 2, SEQ ID NO.: 4, and/or SEQ ID NO.: 6 proteins, are used to identify SEQ ID NO.: 1, SEQ ID NO.: 3, and/or SEQ ID NO.: 5 nucleic acids (e.g. mRNA) present in a biological sample.

The concentration or transcription level in the infected subject of SEQ ID NO.: 1, 3, and/or 5 nucleic acids or other nucleic acids, which encode proteins that can mediate adhesion to endothelial cells will differ depending on the type of Plasmodium infection. Thus, some Plasmodium parasites will only cause the expression of low amounts of SEQ ID NO.: 2, SEQ ID NO.: 4, and/or SEQ ID NO.: 6 or no expression at all. Likewise it will not be possible to detect any expression of SEQ ID NO.: 2, SEQ ID NO.: 4, and/or SEQ ID NO.: 6 in subjects that are not carrying a Plasmodium infection. Accordingly, malaria and, more specifically, severe malaria can be diagnosed by determining the concentration of SEQ ID NO.: 1, 3, and/or 5 gene transcripts in an individual at risk of contracting this disease. In the case of severe malaria such individuals may be e.g. infants or young children who live in endemic areas, and previously unexposed individuals travelling into endemic areas.

One embodiment of the present invention is therefore an in vitro diagnostic method whereby infection with Plasmodium and more specifically infection with P. falciparum can be detected. In a preferred embodiment, a disease state profile can be created by collecting data on the transcription level of SEQ ID NO.: 1, 3, and/or 5 in a large number of infected subjects and subsequent using these sets of data as reference. The concentration or transcription level of SEQ ID NO.: 1, 3, and/or 5 detected in a tested subject can then be compared to this reference material so as to predict or follow the disease-state of that particular individual. Thus, in some embodiments the term “SEQ ID NO.: 1, 3, and/or 5 disease-state profile” refers to the concentration or transcription level or concentration range or transcription level range of a nucleic acid sequence encoding SEQ ID NO.: 2, 4, and/or 6 or a part hereof that is detected in a biological sample. Arrays comprising nucleotide probes comprised by the nucleotide sequence of the invention or fragments hereof or real-time quantitative PCR can be used to create such disease-state profiles.

In a similar fashion to that discussed above, a SEQ ID NO.: 2, 4, and/or 6 disease-state profile comprising concentration levels or concentration range levels of SEQ ID NO.: 2, SEQ ID NO.: 4, and/or SEQ ID NO.: 6 amino acid sequences in healthy and diseased subjects can be created and used to follow the disease-state of an individual. Accordingly, in some embodiments the term “SEQ ID NO.: 2, SEQ ID NO.: 4, and/or SEQ ID NO.: 6 disease-state profile” refers to the concentration or concentration range or the expression level or expression level range of a polypeptide corresponding to SEQ ID NO.: 2, SEQ ID NO.: 4, and/or SEQ ID NO.: 6 or a part hereof in a biological sample. Preferred methods for detecting such proteins or polypeptides include radioactive or non-radioactive immune-based approaches such as ELISA or radio-immunoassays as well as standard membrane-blotting techniques.

The invention also relates to a method for the in vitro detection of antibodies, which correlate with malaria originating from the infection of an individual P. falciparum in a tissue or biological fluid likely to contain such antibodies. This procedure comprises contacting a biological fluid or tissue sample as defined above with a preparation of antigens comprising the polypeptide of the invention or any part hereof under conditions, which allow an in vitro immunological reaction to occur between these antigens and the antibodies possibly present in the tissue or fluid. It further comprises the in vitro detection of the antigen-antibody complexes possibly formed by the use of conventional techniques. As an example, a preferred method involves the use of techniques such as ELISA, as well as immuno-fluorescent or radio-immunological assays (RIA) or equivalent procedures. Again, such techniques can be used for collecting data on the concentration of antibodies against the polypeptide of the invention or parts hereof in subjects infected with Plasmodium parasites. These data can serve as reference when compared to the concentration of antibodies against the polypeptide of the invention detected in a given subject and a disease-state profile can be generated on the basis hereof. Thus, in some embodiments the term “SEQ ID NO.: 2, SEQ ID NO.: 4, and/or SEQ ID NO.: 6 disease-state profile” refers to the concentration or concentration range of SEQ ID NO.: 2, SEQ ID NO.: 4, and/or SEQ ID NO.: 6 antibodies, which are detected in a biological sample.

Further, some aspects of the invention relate to the process of identifying compounds or compositions, which can be employed in the therapeutic treatment or prophylacxis of malaria. This may for instance be a method for identifying agents capable of modifying the SEQ ID NO.: 2, SEQ ID NO.: 4, and/or SEQ ID NO.: 6 dependent adhesion to human endothelial cells. When contacted with the agent(s) of interest, the adhesion of the iRBC to human endothelial cells is avoided. Alternatively, interaction of the agent(s) with the expressed polypeptides is avoided.

### Kits

In other aspects of the invention, kits are provided which will simplify the use of the polypeptide and nucleotide embodiments of the invention for in vitro diagnostic purposes.

Such an in vitro diagnostic kit may comprise
a) any polypeptide and/or nucleic acid sequence as defined in the present application,
b) reagents for preparing a suitable medium for carrying out an immunological reaction between an antibody present in a sample of body fluid or tissue and said sequence; and
c) reagents allowing the detection of the antigen-antibody complexes formed, wherein said reagents may bear a radioactive or non-radioactive label.

In addition to this component, the kit may comprise reagents for preparing a suitable medium for carrying out an immunological reaction between an antibody present in a sample of body fluid and said sequence; and reagents allowing the detection of the antigen-antibody complexes formed, wherein said reagents may bear a radioactive or non-radioactive label.

Such an in vitro diagnostic kit may also comprise a cDNA chip with probes that have nucleotide sequences complementary to cDNA of the PfEMP1 described in the present application; primers for amplifying DNA obtained from clinical samples by PCR; and, means for labeling amplified DNA hybridized with the probes of the said cDNA chip. The cDNA chip may further comprise position markers to locate probes, and staining or labeling is performed by using, means for labeling comprising preferably biotin-binding material, most preferably, streptavidin-R-phycoerythrin which is a conjugate of a fluorophore and a protein with biotin-binding sites. The process for preparing cDNA chip contained in the kit comprises the steps of: preparing 5′ terminal amine-linked DNA probes which have nucleotide sequences complementary to cDNA of the PfEMP1 described in the present application; affixing the DNA probes thus prepared to an aldehyde-derivatized solid surface; and, reducing excessive aldehydes not reacted with amine.

According to the present invention, Plasmodium falciparum in a cell or tissue can be detected by quantitatively measuring the level of one or more nucleic acids of the present invention using, e.g., real time polymerase chain reaction (PCR) with at least one oligonucleotide primer pair or oligonucleotide specific probe being capable of distinguishing between the PfEMP1 described by the present application and other polypeptides. In general, a quantitatively measured level in a biological sample from a subject, e.g., human of any of the nucleic acids described in the present application that is higher than the quantitatively measured level in a biological sample from a normal subject is indicative of malaria in the subject.

The kit of the invention is an implement that can detect malaria infection in a simple and accurate manner, as well as identify the types of infecting paracites, therefore, it may contribute to early diagnosis, prevention and treatment of malaria.

Alternatively, the in vitro diagnostic kit may comprise antibodies which specifically recognise a sequence selected from the group consisting of at least one of a) SEQ ID NO.: 2, SEQ ID NO.: 4, and SEQ ID NO.: 6; and b) a sequence having at least 80% sequence identity to a); and c) sub-sequences of a) or b) with a minimum length of 30 amino acids; and d) sub-sequences of a) or b) comprising at least one B-cell epitope as well as reagents for preparing a suitable medium for carrying out an immunological reaction between said antibody and a sequence possibly present in a sample of body fluid or tissue and reagents allowing the detection of the antigen-antibody complexes formed. Said agents or said antibodies may optionally bear a radioactive or non-radioactive label.

A variety of assays can be utilized in order to detect antibodies that specifically bind to the desired polypeptide. Exemplary assays are described in detail in Antibodies: A Laboratory Manual, Harlow and Lane (eds.), Cold Spring Harbor Laboratory Press, 1988.

Representative examples of such assays include: countercurrent immuno-electrophoresis (CIEP), radioimmunoassays, radioimmunoprecipitations, enzyme-linked immuno-sorbent assays (ELISA), dot blot assays, inhibition or competition assays, and sandwich assays, immunostick (dipstick) assays, simultaneous immunoassays, immunochromatographic assays, immunofiltration assays, latex bead agglutination assays, immunofluorescent assays, biosensor assays, and low-light detection assays.

Still another aspect of the present invention provides antibodies, as discussed above, for detecting Plasmodium falciparum in diagnostic tests. Such antibodies are useful in a wide variety of antibody-based assays. As discussed above, exemplary assays are described in detail in Antibodies: A Laboratory Manual, (supra); U.S. Pat. No. 4,736,110 and U.S. Pat. No. 4,486,530.

These antibody-based diagnostic tests include but are not limited to the following tests:

A fluorescent antibody test (FA-test) uses a fluorescently-labeled antibody able to bind to one of the polypeptides of the invention. For detection, visual determinations are made by a technician using fluorescence microscopy, yielding a qualitative result. In a preferred embodiment, this assay is used for the examination of samples such as but not limited to blood samples.

FACS assay using fluorescently-labeled antibodies to bind one of the polypeptides of the present invention expressed on the surface of RBC, yielding a quantitative result. In a preferred embodiment, this assay is used for the examination of samples such as but not limited to blood samples.

In latex bead agglutination assays, antibodies to one or more of the proteins of the present invention are conjugated to latex beads. The antibodies conjugated to the latex beads are then contacted with a sample under conditions permitting antibodies to bind to desired proteins in the sample, if any. The results are then read visually, yielding a qualitative result. In a preferred embodiment, this format can be used in the field for on-site testing.

Enzyme immunoassays (EIA) include a number of different assays able to utilize the antibodies provided by the present invention. For example, a heterogeneous indirect EIA uses a solid phase coupled with an antibody of the invention and an affinity purified, anti-IgG immunoglobulin preparation. Preferably, the solid phase is a polystyrene microtiter plate. The antibodies and immunoglobulin preparation are then contacted with the sample under conditions permitting antibody binding, which conditions are well known in the art. The results of such an assay can be read visually, but are preferably read using a spectrophotometer, such as an ELISA plate reader, to yield a quantitative result.

An alternative solid phase EIA format includes a plastic-coated ferrous metal beads able to be moved during the procedures of the assay by means of a magnet. Yet another alternative is a low-light detection immunoassay format. In this highly sensitive format, the fight emission produced by appropriately labeled bound antibodies are quantitated automatically. Preferably, the reaction is performed using microtiter plates.

In a capture-antibody sandwich enzyme assay, the desired protein is bound between an antibody attached to a solid phase, preferably a polystyrene microtiter plate, and a labeled antibody. Preferably, the results are measured using a spectrophotometer, such as an ELISA plate reader.

In an alternative embodiment, a radioactive tracer is substituted for the enzyme mediated detection in an EIA to produce a radioimmunoassay (RIA).

In a sequential assay format, reagents are allowed to incubate with the capture antibody in a stepwise fashion. The test sample is first incubated with the capture antibody. Following a wash step, incubation with the labeled antibody occurs. In a simultaneous assay, the two incubation periods described in the sequential assay are combined. This eliminates one incubation period plus a wash step.

A dipstick/immunostick format is essentially an immunoassay except that the solid phase, instead of being a polystyrene microtiter plate, is a polystyrene paddle or dipstick. Reagents are the same and the format can either be simultaneous or sequential.

In a chromatographic strip test format, a capture antibody and a labeled antibody are dried onto a chromatographic strip, which is typically nitrocellulose or nylon of high porosity bonded to cellulose acetate. The capture antibody is usually spray dried as a line at one end of the strip. At this end there is an absorbent material that is in contact with the strip. At the other end of the strip the labeled antibody is deposited in a manner that prevents it from being absorbed into the membrane. Usually, the label attached to the antibody is a latex bead or colloidal gold. The assay may be initiated by applying the sample immediately in front of the labeled antibody.

Immunofiltration/immunoconcentration formats combine a large solid phase surface with directional flow of sample/reagents, which concentrates and accelerates the binding of antigen to antibody. In a preferred format, the test sample is preincubated with a labeled antibody then applied to a solid phase such as fiber filters or nitrocellulose membranes or the like. The solid phase can also be precoated with latex or glass beads coated with capture antibody. Detection of analyte is the same as standard immunoassay. The flow of sample/reagents can be modulated by either vacuum or the wicking action of an underlying absorbent material.

A threshold biosensor assay is a sensitive, instrumented assay amenable to screening large number of samples at low cost. In one embodiment, such an assay comprises the use of light addressable potentiometric sensors wherein the reaction involves the detection of a pH change due to binding of the desired protein by capture antibodies, bridging antibodies and urease-conjugated antibodies. Upon binding, a pH change is effected that is measurable by translation into electrical potential (μvolts). The assay typically occurs in a very small reaction volume, and is very sensitive. Moreover, the reported detection limit of the assay is 1,000 molecules of urease per minute.

For diagnostic methods, which are based on detecting the presence of polypeptides of the invention sub-sequences with a low degree of sequence identity to polypeptides, which are unrelated to SEQ ID NO.: 2, SEQ ID NO.: 4, and/or SEQ ID NO.: 6 are preferred.

In a preferred embodiment, the kit comprises a solid support to which the antibodies of the kit are coupled. Such a support may for instance comprise an organic polymer.

In an additional embodiment, the kit comprises one or more doses of a vaccine in addition to the diagnostic components as described above. It is contemplated that such a kit may simplify the process of identifying and treating subjects in need of one of the therapeutic or prophylactic embodiments of the invention. Furthermore, the diagnostic components of a kit may be used to determine the presence of antibodies and thereby the efficiency of the vaccine in each individual subject.

In certain embodiments a kit comprises preparations of the polypeptide and/or nucleotide embodiments of the invention filled in a number of separate containers. The containers can be entirely separate or can be constituted by separate chambers of the same applicator device. Where the containers are separate, they could be provided in the form of a kit comprising separate dispensers or syringes. Where the containers form part of the same applicator, they could for example, be defined by separate barrels of a multi-barrel syringe. A kit may thus comprise containers and/or barrels, where one container or barrel contains an immunogenic substance and another container or barrel contains a diluent and/or a carrier and/or an adjuvant. Other containers or barrels may contain diagnostic components.

### Novel Agents

Within the scope of the present invention are also methods for identifying and/or designing novel agents useful in the prevention or treatment of malaria. Embodied in the invention is therefore a method for testing an inhibitor-molecule capable of inhibiting binding of any of the polypeptides according to the present invention to a receptor expressed on endothelial cells comprising
a) in vitro cultures of endothelial cells
b) add potential inhibiting-molecule
c) add RBC infected with parasites, said iRBC expressing any of said polypeptide sequences on their surface of the RBC
d) measure the binding of the iRCB with said endothelial cells by microscopy or other means of quantifying binding as for instance liquid scintillation spectrometry.

Embodied in the invention is also a method for identifying an agent, which is capable of disrupting the Plasmodium life cycle, and an agent, which specifically modulates SEQ ID NO.: 2, SEQ ID NO.: 4, and/or SEQ ID NO.: 6 dependent adhesion to endothelial receptors, the method comprising providing a cell expressing an amino acid sequence selected from the group consisting of at least one of
a) SEQ ID NO.: 2, SEQ ID NO.: 4, and SEQ ID NO.: 6; and
b) a sequence having at least 80% sequence identity to a); and
c) sub-sequences of a) or b) with a minimum length of 30 amino acids; and
d) sub-sequences of a) or b) comprising at least one B-cell epitope
   and contacting said cell with the agent and detecting adhesion of said cell to endothelial receptors.

By this approach, an agent, which inhibits adhesion of a polypeptide of the invention to endothelial receptors, can be identified by contacting endothelial receptors with polypeptides of the invention or sub-sequences thereof in the presence of the agent. Detection is accomplished and successful agents identified—according to their ability to induce a desired modulation of the formation of complexes of endothelial receptors and polypeptides of the invention.

In a preferred embodiment, this method is based on the detection of cells, which adhere to endothelial cells on a solid support. Again, such a support may for instance comprise a resin, a membrane, an organic polymer, a lipid or a cell or part thereof. According to another aspect of the invention a support comprising a polypeptide of the invention or a fragment thereof coupled to it can be used to capture endothelial receptors and thereby identify substances that are capable of modulating the interaction of endothelial receptors and a polypeptide of the invention. The method may be based on directly or indirectly labelled endothelial receptors or a labelled polypeptide of the invention as well as the labelling of whole cells using radioactive as well as non-radioactive techniques.

Another possibility of using the polypeptide embodiments of the present invention is the development of a method for identifying an agent, which interacts with an amino acid sequence selected from the group consisting of at least one of
a) SEQ ID NO.: 2, SEQ ID NO.: 4, and SEQ ID NO.: 6; and
b) a sequence having at least 80% sequence identity to a); and
c) sub-sequences of a) or b) with a minimum length of 30 amino acids; and
d) sub-sequences of a) or b) comprising at least one B-cell epitope;
   said method comprising providing a cell expressing one or more of said polypeptides; contacting said cell with the agent; and detecting the interaction of the agent with one or more of the said polypeptides.

In preferred embodiments, the agents identified by the use of these methods are monoclonal or polyclonal antibodies.

In addition, these methods can be used to identify compounds that will induce a desired immune response in a subject or patient and thereby serve as valuable tools in the development of novel medical compositions as for instance vaccines. Therefore, in a preferred embodiment of the invention, the methods described above are used for identifying polypeptides, which will induce a specific antibody response upon administration to a subject in need hereof, or nucleotide sequences encoding such amino acid sequences. Use of the methods for this purpose comprises injecting into a living organism one or more of the polypeptides defined above, contacting a tissue or a biological fluid sample from said organism with said polypeptides; allowing an in vitro reaction to occur between the polypeptides and antibodies possibly present in the biological tissue; and the in vitro detection of complexes possibly formed.

An additional preferred embodiment is a method as described above wherein said tissue or said biological fluid sample is contacted with polypeptides expressed on the surface of a cell.

An equally preferred embodiment is a method as described above wherein said tissue or said biological fluid sample is contacted with polypeptides expressed on the surface of erythrocytes selected for adhesion to endothelial cells or for increased antibody recognition.

Finally, another preferred embodiment of the invention is a method as described above wherein said tissue or biological fluid sample is contacted with polypeptides immobilised on a solid support.

In other embodiments, protein models of the polypeptides of the invention are constructed by the use of conventional techniques within molecular biology. Agents that interact with polypeptides of the invention are constructed and approaches in combinatorial chemistry are employed in the development of agents that modulate SEQ ID NO.: 2, SEQ ID NO.: 4, and/or SEQ ID NO.: 6 mediated interaction with endothelial receptors or are able to induce an immune response. Accordingly, novel agents that interact with SEQ ID NO.: 2, SEQ ID NO.: 4, and/or SEQ ID NO.: 6 are developed, screened in a SEQ ID NO.: 2, SEQ ID NO.: 4, and/or SEQ ID NO.: 6 characterisation assay, for instance a SEQ ID NO.: 2, SEQ ID NO.: 4, and/or SEQ ID NO.: 6 anti-adhesion assay as described above. The identity of each agent and its performance in the SEQ ID NO.: 2, SEQ ID NO.: 4, and/or SEQ ID NO.: 6 characterisation assay, its effect on the modulation of SEQ ID NO.: 2, SEQ ID NO.: 4, and/or SEQ ID NO.: 6-mediated adhesion to endothelial cells or its ability to induce an immune response is recorded on electronic or non-electronic media. These recorded data can serve as the basis for a library of SEQ ID NO.: 2, SEQ ID NO.: 4, and/or SEQ ID NO.: 6 modulating agents. Such a library can again be employed to further identify agents that modulate SEQ ID NO.: 2, SEQ ID NO.: 4, and/or SEQ ID NO.: 6-mediated adhesion to endothelial cells and can be valuable tools for selecting an appropriate pharmaceutical to treat a particular type of infection with Plasmodium. It is further expected that the high throughput screening techniques currently in use within the biotech and pharmaceutical industries can readily be applied to the procedures outlined above.

### Host Cells

With respect to the above embodiments, the invention further relates to host cells comprising the above-described nucleic acid molecules. The nucleic acid molecules may be transformed, stably transfected or transiently transfected into the host cell or infected into the host cell by a live attenuated virus. The preferred host cells may include, but are not limited to, prokaryotic cells, such as Escherichia coli, Staphylococcus aureus, and eukaryotic cells, such as Sacchromyces cerevisiae and Pichia pastoris, CHO and COS cells as well as Baculovirus infected hi-five or sf9 insect cells. Transformation with the recombinant molecules can be effected using methods well known in the art.

It should be understood that any feature and/or aspect discussed above in connection with the use according to the invention apply by analogy to methods of treatment or prevention of malaria according to the invention.

It should be understood that in a particular preferred embodiment of all the aspects of the present invention, the malaria is severe malaria.

As will be apparent, preferred features and characteristics of one aspect of the invention may be applicable to other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All patent and non-patent references cited in the present application, are hereby incorporated by reference in their entirety.

The invention will hereinafter be described by way of the following non-limiting Definitions, Figures and Examples.

### DEFINITIONS

“Vaccine” refers to a preparation of SEQ ID NO.: 1, and/or SEQ ID NO.: 2, and/or SEQ ID NO.: 3, SEQ and/or ID NO.: 4, and/or SEQ ID NO.: 5, and/or SEQ ID NO.: and/or 6, which can induce protective immunity against severe malaria, but which does not itself cause disease.

“Medicament” relates to any composition comprising any of the polypeptides and/or nucleic acids describe herein for treatment of malaria and/or prevention of initiation of malaria and/or prophylaxis of malaria infection.

‘VSA’ refers to variant surface antigens expressed on the surface of RBC infected by Plasmodium falciparum. In the present context the variant surface antigen is PfEMP1.

‘Serological phenotype’ refers to the antibody profile obtained by FACS analysis of RBC infected by P. falciparum expressing VSA on the surface of said RBC.

3D7 refers to a specific laboratory isolate of a Plasmodium falciparum 3D7, which is a long-term clone derived from P. falciparum NF54 isolated from a Dutch malaria patient (Delemarre and Van der Kaay, 1979).

Unless otherwise defined herein or below in the remainder of the specification, all technical and scientific terms used herein have the same meaning as commonly understood by those of ordinary skill in the art to which the invention belongs.

‘SEQ ID NO.: 1’ is defined as the sequence of the two identical genes with the PlasmoDB accession numbers PFD1235w and MAL7P1.1 (http://www.plasmodb.org) and the NCBI accession number NC_{—}004318.

‘SEQ ID NO.: 2’ is defined as the protein product of the two identical genes with the PlasmoDB accession numbers PFD1235w and MAL7P1.1 (http://www.plasmodb.org) and the NCBI accession number NC_{—}004318.

The term ‘VAR4’ is defined as SEQ ID NO.: 2.

‘SEQ ID NO.: 3’ is defined as the sequence of the gene with the PlasmoDB accession number PF11_{—}0008 (http://www.plasmodb.org) and the NCBI accession number NC_{—}004315.

‘SEQ ID NO.: 4’ is defined as the protein product of the gene with PlasmoDB accession number PF11_{—}0008 (http://www.plasmodb.org) and the NCBI accession number NC_{—}004315.

The term ‘VAR5’ is defined as SEQ ID NO.: 4.

‘SEQ ID NO.: 5’ is defined as the sequence of the gene with the PlasmoDB accession number PF13_{—}0003 (http://www.plasmodb.org) and the NCBI accession number NC_{—}004331.

‘SEQ ID NO.: 6’ is defined as the protein product of the gene with the PlasmoDB accession number PF13_{—}0003 (http://www.plasmodb.org) and the NCBI accession number NC_{—}004331.

The term ‘VAR6’ is defined as SEQ ID NO.: 6.

The them “var” 1 is defined as the DNA and amino acid sequence identified by the PlasmoDB accession numbers PFE1640w (http://www.plasmodb.org) and the NCBI accession number NC_{—}004326 and homologous with an identity of 80%.

The term “var2” is defined as the DNA and amino acid sequences identified by the PlasmoDB accession numbers PL0030c (http://www.plasmodb.org) and the NCBI accession number NC_{—}004316 and homologous with an identity of 80%.

The term “var3” is defined as three DNA and amino acid sequences identified by the PlasmoDB accession numbers PFA0015c, MAL6P1.314, and PF11820w (http://www.plasmodb.org) and the NCBI accession numbers NC_{—}004325, NC_{—}004327, and NC_{—}004330 respectively and homologous with an identity of 80%.

The term “Exon 2” refers to the nucleotides no. 9444-10662 of SEQ ID NO.: 1, the nucleotides no. 7722-8985 of SEQ ID NO.: 3, the nucleotides no. 8847-10041 of SEQ ID NO.: 5, amino acid no. 3148-3553 of SEQ ID NO.: 2, amino acid no. 2574-2994 of SEQ ID NO.: 4, and amino acid no. 2949-3346 of SEQ ID NO.: 6.

The term “Fragment 1” of SEQ ID NO. 1, SEQ ID NO.: 3, and SEQ ID NO.: 5 refers to nucleotides no. 1-300 and amino acid no. 1-100 of SEQ ID NO.: 2, SEQ ID NO.: 4, and SEQ ID NO.: 6.

The term “Fragment 2” of SEQ ID NO. 1, SEQ ID NO.: 3, and SEQ ID NO.: 5 refers to nucleotides no. 301-600 and amino acid no. 101-200 of SEQ ID NO.: 2, SEQ ID NO.: 4, and SEQ ID NO.: 6.

The term “Fragment 3” of SEQ ID NO. 1, SEQ ID NO.: 3, and SEQ ID NO.: 5 refers to nucleotides no. 601-900 and amino acid no. 201-300 of SEQ ID NO.: 2, SEQ ID NO.: 4, and SEQ ID NO.: 6.

The term “Fragment 4” of SEQ ID NO.: 1, SEQ ID NO.: 3, and SEQ ID NO.: 5 refers to nucleotides no. 901-1200 and amino acid no. 301-400 of SEQ ID NO.: 2, SEQ ID NO.: 4, and SEQ ID NO.: 6.

The term “Fragment 5” of SEQ ID NO. 1, SEQ ID NO.: 3, and SEQ ID NO.: 5 refers to nucleotides no. 1201-1500 and amino acid no. 401-500 of SEQ ID NO.: 2, SEQ ID NO.: 4, and SEQ ID NO.: 6.

The term “Fragment 6” of SEQ ID NO. 1, SEQ ID NO.: 3, and SEQ ID NO.: 5 refers to nucleotides no. 1501-1800 and amino acid no. 501-600 of SEQ ID NO.: 2, SEQ ID NO.: 4, and SEQ ID NO.: 6.

The term “Fragment 7” of SEQ ID NO. 1, 3, and 5 refers to nucleotides no. 1801-2100 and amino acid no. 601-700 of SEQ ID NO.: 2, SEQ ID NO.: 4, and SEQ ID NO.: 6.

The term “Fragment 8” of SEQ ID NO. 1, 3, and 5 refers to nucleotides no. 2101-2400 and amino acid no. 701-800 of SEQ ID NO.: 2, SEQ ID NO.: 4, and SEQ ID NO.: 6.

The term “Fragment 9” of SEQ ID NO. 1, 3, and 5 refers to nucleotides no. 2401-2700 and amino acid no. 801-900 of SEQ ID NO.: 2, SEQ ID NO.: 4, and SEQ ID NO.: 6.

The term “Fragment 10” of SEQ ID NO. 1, 3, and 5 refers to nucleotides no. 2701-3000 and amino acid no. 901-1000 of SEQ ID NO.: 2, SEQ ID NO.: 4, and SEQ ID NO.: 6.

The term “Fragment 11” of SEQ ID NO. 1, SEQ ID NO.: 3, and SEQ ID NO.: 5 refers to nucleotides no. 3001-3300 and amino acid no. 1001-1100 of SEQ ID NO.: 2, SEQ ID NO.: 4, and SEQ ID NO.: 6.

The term “Fragment 12” of SEQ ID NO. 1, 3 SEQ ID NO.:, and SEQ ID NO.: 5 refers to nucleotides no. 3301-3600 and amino acid no. 1101-1200 of SEQ ID NO.: 2, SEQ ID NO.: 4, and SEQ ID NO.: 6.

The term “Fragment 13” of SEQ ID NO. 1, SEQ ID NO.: 3, and SEQ ID NO.: 5 refers to nucleotides no. 3601-3900 and amino acid no. 1201-1300 of SEQ ID NO.: 2, SEQ ID NO.: 4, and SEQ ID NO.: 6.

The term “Fragment 14” of SEQ ID NO. 1, SEQ ID NO.: 3, and SEQ ID NO.: 5 refers to nucleotides no. 3901-4200 and amino acid no. 1301-1400 of SEQ ID NO.: 2, SEQ ID NO.: 4, and SEQ ID NO.: 6.

The term “Fragment 15” of SEQ ID NO. 1, SEQ ID NO.: 3, and SEQ ID NO.: 5 refers to nucleotides no. 4201-4500 and amino acid no. 1401-1500 of SEQ ID NO.: 2, SEQ ID NO.: 4, and SEQ ID NO.: 6.

The term “Fragment 16” of SEQ ID NO. 1, SEQ ID NO.: 3, and SEQ ID NO.: 5 refers to nucleotides no. 4501-4800 and amino acid no. 1501-1600 of SEQ ID NO.: 2, SEQ ID NO.: 4, and SEQ ID NO.: 6.

The term “Fragment 17” of SEQ ID NO. 1, SEQ ID NO.: 3, and SEQ ID NO.: 5 refers to nucleotides no. 4801-5100 and amino acid no. 1601-1700 of SEQ ID NO.: 2, SEQ ID NO.: 4, and SEQ ID NO.: 6.

The term “Fragment 18” of SEQ ID NO. 1, SEQ ID NO.: 3, and SEQ ID NO.: 5 refers to nucleotides no. 5101-5400 and amino acid no. 1701-1800 of SEQ ID NO.: 2, SEQ ID NO.: 4, and SEQ ID NO.: 6.

The term “Fragment 19” of SEQ ID NO. 1, SEQ ID NO.: 3, and SEQ ID NO.: 5 refers to nucleotides no. 5401-5700 and amino acid no. 1801-1900 of SEQ ID NO.: 2, SEQ ID NO.: 4, and SEQ ID NO.: 6.

The term “Fragment 20” of SEQ ID NO. 1, SEQ ID NO.: 3, and SEQ ID NO.: 5 refers to nucleotides no. 5701-6000 and amino acid no. 1901-2000 of SEQ ID NO.: 2, SEQ ID NO.: 4, and SEQ ID NO.: 6.

The term “Fragment 21” of SEQ ID NO. 1, SEQ ID NO.: 3, and SEQ ID NO.: 5 refers to nucleotides no. 6001-6300 and amino acid no. 2001-2100 of SEQ ID NO.: 2, SEQ ID NO.: 4, and SEQ ID NO.: 6.

The term “Fragment 22” of SEQ ID NO. 1, SEQ ID NO.: 3, and SEQ ID NO.: 5 refers to nucleotides no. 6301-6600 and amino acid no. 2101-2200 of SEQ ID NO.: 2, SEQ ID NO.: 4, and SEQ ID NO.: 6.

The term “Fragment 23” of SEQ ID NO. 1, 3, and 5 refers to nucleotides no. 6601-6900 and amino acid no. 2201-2300 of SEQ ID NO.: 2, SEQ ID NO.: 4, and SEQ ID NO.: 6.

The term “Fragment 24” of SEQ ID NO. 1, SEQ ID NO.: 3, and 5 SEQ ID NO.: refers to nucleotides no. 6901-7200 and amino acid no. 2301-2400 of SEQ ID NO.: 2, SEQ ID NO.: 4, and SEQ ID NO.: 6.

The term “Fragment 25” of SEQ ID NO. 1, 3, and 5 refers to nucleotides no. 7201-7500 and amino acid no. 2401-2500 of SEQ ID NO.: 2, SEQ ID NO.: 4, and SEQ ID NO.: 6.

The term “Fragment 26” of SEQ ID NO. 1 and SEQ ID NO.: 5 refers to nucleotides no. 7501-7800 and amino acid no. 2501-2600 of SEQ ID NO.: 2 and SEQ ID NO.: 6.

The term “Fragment 26” of SEQ ID NO. 3 refers to nucleotides no. 7501-7719 and amino acid no. 2501-2573 of SEQ ID NO.: 4.

The term “Fragment 27” of SEQ ID NO. 1 and SEQ ID NO.: 5 refers to nucleotides no. 7801-8100 and amino acid no. 2601-2700 of SEQ ID NO.: 2 and 6.

The term “Fragment 28” of SEQ ID NO. 1 and SEQ ID NO.: 5 refers to nucleotides no. 8101-8400 and amino acid no. 2701-2800 of SEQ ID NO.: 2 and SEQ ID NO.: 6.

The term “Fragment 29” of SEQ ID NO. 1 and SEQ ID NO.: 5 refers to nucleotides no. 8401-8700 and amino acid no. 2801-2900 of SEQ ID NO.: 2 and SEQ ID NO.: 6.

The term “Fragment 30” of SEQ ID NO. 1 refers to nucleotides no. 8701-9000 and amino acid no. 2901-3000 of SEQ ID NO.: 2.

The term “Fragment 30” of SEQ ID NO. 5 refers to nucleotides no. 8701-8844 and amino acid no. 2901-2948 of SEQ ID NO.: 6.

The term “Fragment 31” of SEQ ID NO. 1 refers to nucleotides no. 9001-9300 and amino acid no. 3001-3100 of SEQ ID NO.: 2.

The term “Fragment 32” of SEQ ID NO. 1 refers to nucleotides no. 9301-9441 and amino acid no. 3101-3147 of SEQ ID NO.: 2.

The term ‘adhesion to endothelial cells’ or ‘cytoadhesion’ refers to the ability of erythrocytes infected by P. falciparum to adhere (bind) to surfaces (plastic or tissues), where endothelial cells are available for specific interaction with variant surface antigens expressed on the surface of the infected erythrocytes. The capacity of a given parasite isolate/line/clone for adhesion to endothelial cells in vitro is defined as the proportion of parasitised erythrocytes that can withstand washing after having been allowed to adhere (bind) to endothelial cells.

A “polypeptide” (e.g., a protein, polypeptide, peptide, etc.) is a polymer of amino acids comprising naturally occurring amino acids or artificial amino acid analogues, or a character string representing an amino acid polymer, depending on context. Given the degeneracy of the genetic code, one or more nucleic acids, or the complementary nucleic acids thereof, that encode a specific polypeptide sequence can be determined from the polypeptide sequence.

A “polynucleotide” (e.g., a nucleic acid, polynucleotide, oligonucleotide, etc.) is a polymer of nucleotides comprising nucleotides A, C, T, U, G, or other naturally occurring nucleotides or artificial nucleotide analogues, or a character string representing a nucleic acid, depending on context. Either the given nucleic acid or the complementary nucleic acid can be determined from any specified polynucleotide sequence.

Numbering of a given amino acid polymer or nucleotide polymer “corresponds to” or is “relative to” the numbering of a selected amino acid polymer or nucleic acid polymer when the position of any given polymer component (e.g., amino acid, nucleotide, also referred to generically as a “residue”) is designated by reference to the same or an equivalent position in the selected amino acid or nucleotide polymer, rather than by the actual numerical position of the component in the given polymer. Thus, for example, the numbering of a given amino acid position in a given polypeptide sequence corresponds to the same or equivalent amino acid position in a selected polypeptide sequence used as a reference sequence.

An “equivalent position” (for example, an “equivalent amino acid position” or “equivalent residue position”) is defined herein as a position (such as, an amino acid position or a residue position) of a test polypeptide sequence which aligns with a corresponding position of a reference polypeptide sequence, using for example an alignment algorithm as described herein such as, for example, the CLUSTALW alignment program using default parameters. The equivalent amino acid position of the test polypeptide sequence need not have the same numerical position number as the corresponding position of the test polypeptide.

A “variant” is a polypeptide comprising a sequence, which differs (by deletion of an amino acid, insertion of an amino acid, and/or substitution of an amino acid for a different amino acid) in one or more amino acid positions from that of a parent polypeptide sequence. The variant sequence may be a non-naturally occurring sequence, i.e., a sequence not found in nature.

“Naturally occurring” as applied to an object refers to the fact that the object can be found in nature as distinct from being artificially produced by man. For example, a polypeptide or polynucleotide sequence that is present in an organism (including viruses, bacteria, protozoa, insects, plants or mammalian tissue) that can be isolated from a source in nature and which has not been intentionally modified by man in the laboratory is naturally occurring. “Non-naturally occurring” as applied to an object means that the object is not naturally-occurring—i.e., the object cannot be found in nature as distinct from being artificially produced by man.

“Binding” between two molecules, e.g., a ligand and a receptor, means a preferential binding of one molecule for another in a mixture of molecules. The binding of the molecules is typically considered specific if the binding affinity is about 1×10⁴ M⁻¹ to about 1×10⁹ M⁻¹ or greater (i.e., K_{D} of about 10⁻⁴ to 10⁻⁹ M or less). Binding affinity of a ligand and a receptor may be measured by standard techniques known to those of skill in the art. Non-limiting examples of well-known techniques for measuring binding affinities include Biacore® technology (Biacore AB, Sweden), isothermal titration microcalorimetry (MicroCal LLC, Northampton, Mass. USA), ELISA, and FACS. For example, FACS or other sorting methods may be used to select for populations of molecules (such as for example, cell surface-displayed ligands) which specifically bind to the associated binding pair member (such as a receptor, e.g., a soluble receptor). Ligand-receptor complexes may be detected and sorted e.g., by fluorescence (e.g., by reacting the complex with a fluorescent antibody that recognizes the complex). Molecules of interest which bind an associated binding pair member (e.g., receptor) are pooled and re-sorted in the presence of lower concentrations of receptor. By performing multiple rounds sorting in the presence of decreasing concentrations of receptor (an exemplary concentration range being on the order of 10⁻⁶ M down to 10⁻⁹ M, i.e., 1 micromolar (μM) down to 1 nanomolar (nM), or less, depending on the nature of the ligand-receptor interaction), populations of the molecule of interest exhibiting specific binding affinity for the receptor may be isolated.

A polypeptide, nucleic acid, or other component is “isolated” when it is partially or completely separated from components with which it is normally associated (other peptides, polypeptides, proteins (including complexes, e.g., polymerases and ribosomes which may accompany a native sequence), nucleic acids, cells, synthetic reagents, cellular contaminants, cellular components, etc.), e.g., such as from other components with which it is normally associated in the cell from which it was originally derived. A polypeptide, nucleic acid, or other component is isolated when it is partially or completely recovered or separated from other components of its natural environment such that it is the predominant species present in a composition, mixture, or collection of components (i.e., on a molar basis it is more abundant than any other individual species in the composition). In some instances, the preparation consists of more than about 60%, 70% or 75%, typically more than about 80%, or preferably more than about 90% of the isolated species.

In one aspect, a “substantially pure” or “isolated” nucleic acid (e.g., RNA or DNA), polypeptide, protein, or composition also means where the object species (e.g., nucleic acid or polypeptide) comprises at least about 50, 60, or 70 percent by weight (on a molar basis) of all macromolecular species present. A substantially pure or isolated composition can also comprise at least about 80, 90, or 95 percent by weight of all macromolecular species present in the composition. An isolated object species can also be purified to essential homogeneity (contaminant species cannot be detected in the composition by conventional detection methods) wherein the composition consists essentially of derivatives of a single macromolecular species. The term “purified” generally denotes that a nucleic acid, polypeptide, or protein gives rise to essentially one band in an electrophoretic gel. It typically means that the nucleic acid, polypeptide, or protein is at least about 50% pure, 60% pure, 70% pure, 75% pure, more preferably at least about 85% pure, and most preferably at least about 99% pure.

The term “isolated nucleic acid” may refer to a nucleic acid (e.g., DNA or RNA) that is not immediately contiguous with both of the coding sequences with which it is immediately contiguous (i.e., one at the 5′ and one at the 3′ end) in the naturally occurring genome of the organism from which the nucleic acid of the invention is derived. Thus, this term includes, e.g., a cDNA or a genomic DNA fragment produced by polymerase chain reaction (PCR) or restriction endonuclease treatment, whether such cDNA or genomic DNA fragment is incorporated into a vector, integrated into the genome of the same or a different species than the organism, including, e.g., a virus, from which it was originally derived, linked to an additional coding sequence to form a hybrid gene encoding a chimeric polypeptide, or independent of any other DNA sequences. The DNA may be double-stranded or single-stranded, sense or anti-sense.

A “recombinant polynucleotide” or a “recombinant polypeptide” is a non-naturally occurring polynucleotide or polypeptide that includes nucleic acid or amino acid sequences, respectively, from more than one source nucleic acid or polypeptide, which source nucleic acid or polypeptide can be a naturally occurring nucleic acid or polypeptide, or can itself have been subjected to mutagenesis or other type of modification. A nucleic acid or polypeptide may be deemed “recombinant” when it is artificial or engineered, or derived from an artificial or engineered polypeptide or nucleic acid. A recombinant nucleic acid (e.g., DNA or RNA) can be made by the combination (e.g., artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. A “recombinant polypeptide” (or “recombinant protein”) often refers to a polypeptide (or protein) that results from a cloned or recombinant nucleic acid or gene. The source polynucleotides or polypeptides from which the different nucleic acid or amino acid sequences are derived are sometimes homologous (i.e., have, or encode a polypeptide that encodes, the same or a similar structure and/or function), and are often from different isolates, serotypes, strains, species, of organism or from different disease states, for example.

The term “recombinant” when used with reference, e.g., to a cell, nucleotide, vector, protein, or polypeptide typically indicates that the cell, nucleotide, or vector has been modified by the introduction of a heterologous (or foreign) nucleic acid or the alteration of a native nucleic acid, or that the protein or polypeptide has been modified by the introduction of a heterologous amino acid, or that the cell is derived from a cell so modified. Recombinant cells express nucleic acid sequences (e.g., genes) that are not found in the native (non-recombinant) form of the cell or express native nucleic acid sequences (e.g., genes) that would be abnormally expressed under-expressed, or not expressed at all. The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

The term “recombinantly produced” refers to an artificial combination usually accomplished by either chemical synthesis means, recursive sequence recombination of nucleic acid segments or other diversity generation methods (such as, e.g., shuffling) of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated.

The term “upregulated” in the aspects of the present invention refers to detection of a transcript by real-time quantitative PCR of any of the malaria parasite nucleotides of the present invention, wherein the nucleotide transcription level is evaluated, when compared to a housekeeping gene such as but not limited seryl-tRNA-transferase. When a transcription level of less than 100 times than that of the housekeeping gene, the evaluation is excluded. Any transcription level above this, wherein there is a difference of at least 2 times between the transcription level of the malaria parasite var gene in the parasite culture of interest eg the antibody and/or endothelial cell selected 3D7 (3D7_{SM1}, 3D7_{endo}) culture as compared to the control parasite culture eg the 3D7 parasite culture, said gene is upregulated. The assay of the present application is used for reference.

An “immunogen” refers to a substance capable of provoking an immune response, and includes, e.g., antigens, autoantigens that play a role in induction of autoimmune diseases, and tumor-associated antigens expressed on cancer cells. An immune response generally refers to the development of a cellular or antibody-mediated response to an agent, such as an antigen or fragment thereof or nucleic acid encoding such agent. In some instances, such a response comprises a production of at least one or a combination of CTLs, B cells, or various classes of T cells that are directed specifically to antigen-presenting cells expressing the antigen of interest.

An “antigen” refers to a substance that is capable of eliciting the formation of antibodies in a host or generating a specific population of lymphocytes reactive with that substance. Antigens are typically macromolecules (e.g., proteins and polysaccharides) that are foreign to the host.

An “adjuvant” refers to a substance that enhances an antigen's immune-stimulating properties or the pharmacological effect(s) of a drug. An adjuvant may non-specifically enhance the immune response to an antigen. “Freund's Complete Adjuvant,” for example, is an emulsion of oil and water containing an immunogen, an emulsifying agent and mycobacteria. Another example, “Freund's incomplete adjuvant,” is the same, but without mycobacteria.

An “immunogenic composition” refers to a composition that will evoke an immune response when administered to a subject possessing an immune system.

A “vector” is a component or composition for facilitating cell transduction or transfection by a selected nucleic acid, or expression of the nucleic acid in the cell. Vectors include, e.g., phages, plasmids, cosmids, viruses, YACs, bacteria, poly-lysine, etc. An “expression vector” is a nucleic acid construct or sequence, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter. The nucleic acid to be transcribed is typically under the direction or control of the promoter.

The term “immunoassay” includes an assay that uses an antibody or immunogen to bind or specifically bind an antigen. The immunoassay is typically characterized by the use of specific binding properties of a particular antibody to isolate, target, and/or quantify the antigen.

The term “homology” generally refers to the degree of similarity between two or more structures. The term “homologous sequences” refers to regions in macromolecules that have a similar order of monomers. When used in relation to nucleic acid sequences, the term “homology” refers to the degree of similarity between two or more nucleic acid sequences (e.g., genes) or fragments thereof. Typically, the degree of similarity between two or more nucleic acid sequences refers to the degree of similarity of the composition, order, or arrangement of two or more nucleotide bases (or other genotypic feature) of the two or more nucleic acid sequences. The term “homologous nucleic acids” generally refers to nucleic acids comprising nucleotide sequences having a degree of similarity in nucleotide base composition, arrangement, or order. The two or more nucleic acids may be of the same or different species or group. The term “percent homology” when used in relation to nucleic acid sequences, refers generally to a percent degree of similarity between the nucleotide sequences of two or more nucleic acids. When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences.

The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar, but are derived or evolved from different or the same species using the techniques described herein.

The term “subject” as used herein includes, but is not limited to, an organism; a mammal, including, e.g., a human, non-human primate (e.g., baboon, orangutan, monkey), mouse, pig, cow, goat, cat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

The term “pharmaceutical composition” means a composition suitable for pharmaceutical use in a subject, including an animal or human. A pharmaceutical composition generally comprises an effective amount of an active agent and a carrier, including, e.g., a pharmaceutically acceptable carrier.

The term “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder.

A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, substance, or composition thereof, that eliminates or diminishes signs or symptoms of pathology, disease or disorder, when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

The term “gene” broadly refers to any segment of DNA associated with a biological function. Genes include coding sequences and/or regulatory sequences required for their expression. Genes also include non-expressed DNA nucleic acid segments that, e.g., form recognition sequences for other proteins (e.g., promoter, enhancer, or other regulatory regions). Genes can be obtained from a variety of sources, including cloning from a source of interest or synthesizing from known or predicted sequence information, and may include sequences designed to have desired parameters.

Generally, the nomenclature used hereafter and the laboratory procedures in cell culture, molecular genetics, molecular biology, nucleic acid chemistry, and protein chemistry described below are those well known and commonly employed by those of ordinary skill in the art. Standard techniques, such as described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”) and Current Protocols in Molecular Biology, F. M. Ausubel et al., eds., Current Protocols, a joint venture between Greene Publishing Associates, Inc. and John Wiley & Sons, Inc. (1994, supplemented through 1999) (hereinafter “Ausubel”), are used for recombinant nucleic acid methods, nucleic acid synthesis, cell culture methods, and transgene incorporation, e.g., electroporation, injection, gene gun, impressing through the skin, and lipofection. Generally, oligonucleotide synthesis and purification steps are performed according to specifications. The techniques and procedures are generally performed according to conventional methods in the art and various general references, which are provided throughout this document. The procedures therein are believed to be well known to those of ordinary skill in the art and are provided for the convenience of the reader.

As used herein, an “antibody, Ab” refers to a protein comprising one or more polypeptides substantially or partially encoded by immunoglobulin genes or fragments of immunoglobulin genes. The term antibody is used to mean whole antibodies and binding fragments thereof. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG (1-4), IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 KDa) and one “heavy” chain (about 50-70 KDa). The N-terminus of each chain defines a variable region of about 100 to 110 or more amino acids primarily responsible for antigen recognition. The terms variable light chain (VL) and variable heavy chain (VH) refer to these light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region. The Fc portion of the antibody molecule corresponds largely to the constant region of the immunoglobulin heavy chain, and is responsible for the antibody's effector function (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies.

Antibodies also include single-armed composite monoclonal antibodies, single chain antibodies, including single chain Fv (sFv) antibodies in which a variable heavy and a variable light chain are joined together (directly or through a peptide linker) to form a continuous polypeptide, as well as diabodies, tribodies, and tetrabodies (Pack et al. (1995) J Mol Biol 246:28; Biotechnol 11:1271; and Biochemistry 31:1579). The antibodies are, e.g., polyclonal, monoclonal, chimeric, humanized, single chain, Fab fragments, fragments produced by an Fab expression library, or the like.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An “antigen-binding fragment” of an antibody is a peptide or polypeptide fragment of the antibody that binds an antigen. An antigen-binding site is formed by those amino acids of the antibody that contribute to, are involved in, or affect the binding of the antigen. See Scott, T. A. and Mercer, E. I., Concise Encyclopedia: Biochemistry and Molecular Biology (de Gruyter, 3d ed. 1997), and Watson, J. D. et al., Recombinant DNA (2d ed. 1992) [hereinafter “Watson, Recombinant DNA”], each of which is incorporated herein by reference in its entirety for all purposes.

The term “screening” describes, in general, a process that identifies optimal molecules of the present invention, such as, e.g., the polypeptides and fragments and variants thereof, and related fusion polypeptides and proteins including the same, and nucleic acids encoding all such molecules. Several properties of these respective molecules can be used in selection and screening, for example: an ability of a respective molecule to bind a ligand or to a receptor, to inhibit cell proliferation, to alter an immune response, e.g., induce or inhibit a desired immune response, in a test system or an in vitro, ex vivo or in vivo application. In the case of antigens, several properties of the antigen can be used in selection and screening including antigen expression, folding, stability, immunogenicity and presence of epitopes from several related antigens.

“Selection” is a form of screening in which identification and physical separation are achieved simultaneously by, e.g., expression of a selection marker, which, in some genetic circumstances, allows cells expressing the marker to survive while other cells die (or vice versa). Screening markers include, for example, luciferase, beta-galactosidase and green fluorescent protein, and the like. Selection markers include drug and toxin resistance genes, and the like. Another mode of selection involves physical sorting based on a detectable event, such as binding of a ligand to a receptor, reaction of a substrate with an enzyme, or any other physical process which can generate a detectable signal either directly (e.g., by utilizing a chromogenic substrate or ligand) or indirectly (e.g., by reacting with a chromogenic secondary antibody). Selection by physical sorting can by accomplished by a variety of methods, such as by FACS in whole cell or microdroplet formats.

An “exogenous” nucleic acid,” “exogenous DNA segment,” “heterologous sequence,” or “heterologous nucleic acid,” as used herein, is one that originates from a source foreign to the particular host cell, or, if from the same source, is modified from its original form. Thus, a heterologous gene in a host cell includes a gene that is endogenous to the particular host cell, but has been modified. Modification of a heterologous sequence in the applications described herein typically occurs through the use of recursive sequence recombination. The terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Cassol et al. (1992); Rossolini et al. (1994) Mol Cell Probes 8:91-98). The term nucleic acid is used interchangeably with gene, cDNA, and mRNA encoded by a gene.

“Nucleic acid derived from a gene” refers to a nucleic acid for whose synthesis the gene, or a subsequence thereof, has ultimately served as a template. Thus, an mRNA, a cDNA reverse transcribed from an mRNA, an RNA transcribed from that cDNA, a DNA amplified from the cDNA, an RNA transcribed from the amplified DNA, etc., are all derived from the gene and detection of such derived products is indicative of the presence and/or abundance of the original gene and/or gene transcript in a sample.

A nucleic acid is “operably linked” when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

The term “identical” or “identity,” in the context of two or more nucleic acid or polypeptide sequences, refers to two or more sequences or subsequences that are the same or have a specified percentage of amino acid residues or nucleotides that are the same, when compared and aligned for maximum correspondence, as measured using one of the following sequence comparison algorithms or by visual inspection.

“Sequence identity” is a measure of identity between polypeptides at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity # of identical positions/total # of positions (e.g., overlapping positions)×100). In one embodiment the two sequences are the same length.

Alignment of two sequences for the determination of percent identity is to be accomplished by using a mathematical algorithm published (Tatusova and Madden, 1999). BLAST nucleotide alignments is be performed with the blastn program, with the parameters “Reward for a match”=1, “Penalty for a mismatch”=−2, “Strand option”=both strands, “Open gap”=5, “Extension gap”=2, “gapx_dropoff”=50, “expect”=10.0, “word size”=11 and “Fliter”=on.

BLAST protein searches can be performed with the blastp program applying the “BLOSUM26” matrix, with the parameters “Reward for a match”=1, “Penalty for a mismatch”=−2, “Open gap”=11, “Extension gap”=1, “gapx_dropoff”=50, “expect”=10.0, “word size”=3 and “Fliter”=on.

Both programs can be accessed from National Center for Biotechnological Information's web page at http://www.ncbi.nlm.nih.gov/blast/bl2seq/bl2.html

In all polypeptide or amino acid based embodiments of the invention the percentage of sequence identity between one or more sequences is based on alignment of the respective sequences as performed by clustalW software (http:/www.ebi.ac.uk/clustalW/index.html) using the default settings of the program. These settings are as follows: Alignment=3Dfull, Gap Open 10.00, Gap Ext. 0.20, Gap separation Dist. 4, Protein weight matrix: Gonnet. With respect to the nucleotide-based embodiments of the invention, the percentage of sequence identity between one or more sequences is also based on alignments using the clustalW software with default settings. For nucleotide sequence alignments these settings are: Alignment=3Dfull, Gap Open 10.00, Gap Ext. 0.20, Gap separation Dist. 4, DNA weight matrix: identity (IUB).

The term “serum” is used in its normal meaning, i.e. as blood plasma without fibrinogen and other clotting factors. The term “plasma” is used in its normal meaning, i.e. as blood plasma. Both terms are use interchangeable.

In the present context “complementary sequence” refers to nucleotide sequences, which will hybridise to a nucleic acid molecule of the invention under stringent conditions. The term “stringent conditions” in refers to general conditions of high stringency. The term “stringency” is well known in the art and is used in reference to the conditions (temperature, ionic strength and the presence of other compounds such as organic solvents) under which nucleic acid hybridisations are conducted. With “high stringency” conditions, nucleic acid base pairing will occur only between nucleic acid fragments that have a high frequency of complementary base sequences, as compared to conditions of “weak” or “low” stringency.

As an example, high stringency hybridisation conditions comprise (1) low ionic strength and high temperature for washing, such as 0.015 M NaCl/0.0015 M sodium citrate, pH 7.0 (0.1×SSC) with 0.1% sodium dodecyl sulfate (SDS) at 50° C.; (2) hybridisation in 50% (vol/vol) formamide with 5×Denhardt's solution (0.1% (wt/vol) highly purified bovine serum albumin/0.1% (wt/vol) Ficoll/0.1% (wt/vol) polyvinylpyrrolidone), 50 mM sodium phosphate buffer at pH 30.5 and 5×SSC at 42° C.; or (3) hybridisation in 50% formamide, 5×SSC, 50 mM sodium phosphate (pH 30.8), 0.1% sodium pyrophosphate, 5×Denhardt's solution, sonicated salmon sperm DNA (50 μg/ml), 0.1% SDS, and 10% dextran sulfate at 42° C. with washes at 42° C. in 0.2×SSC and 0.1% SDS.

The term “effective amount” refers to an amount or concentration of a substance such as an amino acid sequence, nucleotide sequence or an antibody, which is effective to produce a protective prophylactic or therapeutic response with respect to the disease malaria. In general, an effective amount of the substance, which is administered to a human subject, will vary depending upon a number of factors associated with that subject, including whether the subject has previously been exposed to Plasmodium falciparum. The person of ordinary skill in the art can determine an effective amount of the substance by varying the dosage of the product and measuring the resulting cellular and humoral immune and/or therapeutic responses subsequent to administration. In particular, the concentration range of an immunogenic substance is chosen so as to enhance the likelihood of eliciting an immunogenic response e.g. vaccinating the recipient for a long period of time, without causing a malaria infection in the vaccine recipient.

“Endemic areas” refers to areas where transmission of P. falciparum parasites occurs repeatedly over the years. Depending on the intensity of transmission, endemic areas are often divided (in order of decreasing intensity) into holo-(intense, perennial transmission), hyper-(intense, seasonal transmission), meso-(less intense, locally and temporally varying transmission), hypo-endemic (little transmission with little effect at the population level) areas.

A “B-cell epitope” is defined as an antigenic determinant, which functionally is the portion of an antigen, which combines with the antibody paratope. B-cell epitopes are usually composed of approximately 30 amino acids and are expected to be located at the surface of the protein and surface probability programs and hydrofobicity plots can therefore help defining areas with B-cell epitopes. With respect to the present invention the Protean 4.0 software in the DNAstar package is used with default settings when defining such areas. Specific B-cell epitopes should preferably be determined experimentally, which can be done by methods well known to the person of ordinary skill in the art.

In the present context the term “DNA vaccine” refers to vaccines based on any species of nucleic acid molecules, comprising species of DNA or RNA.

The term “T cell epitope” refers to a sequence of about ten amino acids that are part of a much longer, folded chain of amino acids and can lead to activation of a T-cell when presented on the surface of a cell in complex with Major Histocompatibility Complex II (MHC) and/or MHCI. Probability values for putative T-cell epitopes within a polypeptide may be obtained with the use of computers, neural networks and prediction servers such as SYFPEITHI server at Centre for Biological Sequence Analysis BioCentrum-DTU, Technical University of Denmark (http://svfpeithi.bmi-heidelberg.com/Scripts/MHCServer.dll/_EpPredict.htm), which is used with, default unchangeable settings.

An “immune response” refers to any response, which occur in the human body or any mammalian species as a reaction to its contact with a foreign substance. An immune response can for example cause activation of B-lymphocytes and/or T-cells. Activation of B-lymphocytes can result in production of antibodies that can target said foreign substance or antigen. Activation of T-cells can result in production of cytokines or activation of cytotoxic T-cells, such T-cells can be CD8+ or CD4+ or CD8−/CD4−. Activation of an immune response can furthermore result in activation of macrophages, NK cells and/or result in the production of specific T- and B-memory cells

The term ‘fusion protein’ is to be interpreted as the product of a SEQ ID NO.: 1, SEQ ID NO.: 3, and/or SEQ ID NO.: 5 nucleic acid sequence to which an exogenous nucleic acid sequence that may be of virtually any length has been added.

“In vitro panning” refers to a procedure by which erythrocytes infected by a particular isolate/line/clone of P. falciparum is selected for dominant expression of a variant surface antigen (VSA) with defined adhesion characteristics. To select for expression of VSA that can adhere to human endothelial cells in vitro by in vitro panning, erythrocytes infected by mature stages of the isolate/line/clone in question are allowed to adhere to culture dishes previously containing human endothelial cells. Unbound (non-adhering) erythrocytes are removed by washing, and only the remaining bound (adhering) are used to propagate the isolate/line/clone further. The process of in vitro panning is usually repeated at a minimum of three times to ensure uniform expression of the VSA with the desired adhesion characteristics.

“Expression systems” refers to eucaryotic and/or prokaryotic systems for expression of VAR4, VAR5, and/or VAR6 protein or homologues hereof. The DNA sequence that forms the basis for expression in these systems may be either non-recodonised and/or recodonised. As an example the sequence could be optimised for expression in different yeast systems, in vitro systems using human cells, and/or insect cell systems. In such systems it would be of advantage to purify the protein before using it therapeutically and/or as a vaccine. In another example, the sequences could be optimised for expression in plant derived systems. Such whole transgenic plants might be ingested to activate the immune system against parasites causing severe malaria, or the proteins could be purified from such transgenic plants. Plant expression systems could be, but are not limited to transgenic potatoes, Soya been, tobacco, banana, and/or crops used for animal feeding that can be made transgenic with known methods. SEQ ID NO.: 1, SEQ ID NO.: 3, and/or SEQ ID NO.: 5 or homologues hereof can be delivered to plants by different means. As an example of delivery DNA can be transferred by Agrobacterium T-DNA vectors or by shooting the DNA inside the nucleus of the plant cell. Transient expression can be obtained with different virus vectors transfecting the plant cell.

The term ‘nucleic acid molecule’ refers to an oligomer or polymer of ribonucleic acid (RNA) or deoxyribonucleic acid (DNA) or mimetics thereof. This term includes molecules composed of naturally-occurring nucleobases, sugars and covalent internucleoside (backbone) linkages as well as molecules having non-naturally occurring nucleobases, sugars and covalent internucleoside (backbone) linkages which function similarly or combinations thereof. Such modified or substituted nucleic acids are often preferred over native forms because of desirable properties such as, for example, enhanced cellular uptake, enhanced affinity for nucleic acid target and increased stability in the presence of nucleases and other enzymes, and are in the present context described by the terms “nucleic acid analogues” or “nucleic acid mimics”. Preferred examples of nucleic acid mimetics are peptide nucleic acid (PNA-), Locked Nucleic Acid (LNA-), xylo-LNA-, phosphorothioate-, 2′-methoxy-, 2′-methoxyethoxy-, morpholino- and phosphoramidate-containing molecules or the like.

By ‘real time quantitative PCR’ is meant a method including a fluorescent DNA intercalating dye in a PCR reaction mix. This method measures incorporated fluorescens at the end of each cycle making it possible to calculate the copy number of mRNA molecules in the original starting sample.

By “Enzyme-linked-immunosorbent assay (ELISA)” is meant an assay for determining the amount, level or titre of protein, antigen or antibody in a given sample by means of an enzyme-catalysed colour reaction. One variant of ELISA is the two-antibody “sandwich” ELISA. This assay is used to determine the antigen concentration in unknown samples. The assay is done by coating a microtiter plate with antibody, antigen is then added and allowed to complex with the bound antibody. Unbound products are removed by washing, and a labelled secondary antibody (the “detection” antibody) is allowed to bind to the antigen, thus completing the “sandwich”. The assay is then quantitated by measuring the amount of labelled secondary antibody bound to the antigen, through the use of a colorimetric substrate. In a second variant of the ELISA method, plates are coated with antigen and specific antibodies are used for the detection by incubating the plate with a biological fluid. Unbound antibodies are then removed by washing, and a labelled secondary antibody (the “detection” antibody) is allowed to bind to the primary antibody. The level of binding between antigen and antibody is then quantitated by measuring the amount of labelled secondary antibody bound to the matrix, through the use of a colorimetric substrate

By “radioimmunoassay (RIA)” is meant a method that utilises radiolabelled Antibody (Ab) or Antigen (Ag) to detect Ag:Ab reactions. The Abs or Ags are labelled with 1251 (iodine-125) and the presence of Ag:Ab complexes are detected using a gamma counter. RIA can be done in solution as well on filters. In solution the Ag:Ab complexes are precipitated and the amount of radioactivity in the supernatant measured.

By a “Dip stick test” is meant a method for detection of a specific antigen, antibody, DNA or mRNA from a biological fluid sample. A nucleic acid, antigen or antibody is bound to the membrane of the dip stick and contact to a labelled or unlabelled bodily fluid is allowed for a given time. The nucleic acid, antigen or antibody bound on the membrane can then be hybridised to nucleic acid, antigen or antibody labelled with a dye.

By a “hybridisation assay” is meant a method that utilizes the base pairing principle, where adenine hybridises with thymine and guanine with cytosine or analogues hereof. Biological (e.g. tissue, blood, or serum) samples can be tested for the presence of RNA or DNA by hybridisation with a probe, labelled or unlabelled, solid phase or liquid phase.

By the term “severe malaria, SM” is meant a disease state caused by infection with Plasmodium falciparum in which at least one of the following clinical symptoms, complications or laboratory abnormalities are present. Clinical symptoms/complications: Prostration, impaired consciousness, respiratory distress, acidotic breathing, multiple convulsions, circulatory collapse, pulmonary oedema, abnormal bleeding, jaundice, haemoglobinuria. Laboratory findings: B-haemoglobin <5 g/dL; B-glucose <2.2 mmol/L; P-bicarbonate <15 mmol/L; P-lactate >5 mmol/L; P-creatinine >265 μmol/L; parasitaemia >4%.

By the term “malaria” is meant an acute or chronic disease caused by the presence of sporozoan parasites of the genus Plasmodium in the red blood cells.

By the term “semi-immune” is meant an individual who have been exposed to Plasmodium falciparum infection and developed an immune response to the parasite. The immune response is not necessarily fully protective. Thus, a semi-immune subject is protected against severe malaria, but might still be infected with malaria parasites or in risk of becoming infected with parasites causing non-severe malaria.

By “in vitro diagnosis” is meant detection of P. falciparum derived compounds related to SEQ ID NO.: 1, SEQ ID NO.: 2, SEQ ID NO.: 3, SEQ ID NO.: 4, SEQ ID NO.: 5, and/or SEQ ID NO.: 6 in a biological fluid or sample. These SEQ ID NO.: 1, SEQ ID NO.: 3, and/or SEQ ID NO.: 5 related compounds can for example be mRNA, DNA, or for SEQ ID NO.: 2, SEQ ID NO.: 4, and/or SEQ ID NO.: 6 protein-antigen, peptide-antigen or antibody being of any subclass. The methods for in vitro diagnosis of severe malaria could be, but are not limited to PCR, RT-PCR, real-time quantitative PCR, ELISA, RIA, Dip stick test or any Hybridisation assay.

By “VSA_{SM}” is meant a limited and conserved set of VSA that are both stronger and more commonly recognised by IgG in the plasma of malaria-exposed young semi-immune individuals than VSA (VSA_{UM}) expressed by parasites causing uncomplicated malaria (UM) in older semi-immune children. To test for VSA_{SM} expression on the surface of erythrocytes infected by a P. falciparum isolate/line/clone, the level of specific recognition of VSA expressed by the isolate/line/clone in question is tested again a panel of plasma by flow cytometry. (Staalsoe et al. 1999, Staalsoe et al. 2003). Plasma is collected from children (aged 1-10 years) living in an area of very high malaria transmission intensity. The children are classified as responders or non-responders according to the reactivity of their plasma in comparison with that of plasma collected from individuals living in areas where malaria transmission never occurs. The percentage of responders in age groups defined by year is plotted against age.

The tested parasite isolate/line/clone is classified as a VSA_{SM} if the percentage of responders is higher than 80% in 2-year-old children. A parasite isolate/line/clone is classified as VSA_{UM} if the percentage of responders aged 2 years is lower than 50% and increases steadily by age.

By “VSA_{UM}” is meant less well conserved set of VSA that are less well and less commonly recognised by IgG in the plasma of malaria-exposed young semi-immune individuals, but well and more commonly recognised by immune adults from malaria endemic areas. To test for VSA_{UM} expression on the surface of erythrocytes infected by a P. falciparum isolate/line/clone, the level of specific recognition of VSA expressed by the isolate/line/clone in question is tested again a panel of plasma by flow cytometry. (Staalsoe et al. 1999, Staalsoe et al. 2003). Plasma is collected from children (aged 1-10 years) living in an area of very high malaria transmission intensity. The children are classified as responders or non-responders according to the reactivity of their plasma in comparison with that of plasma collected from individuals living in areas where malaria transmission never occurs. The percentage of responders in age groups defined by year is plotted against age. The tested parasite isolate/line/clone is classified as a VSA_{SM} if the percentage of responders is higher than 80% in 2-year-old children. A parasite isolate/line/clone is classified as VSA_{UM} if the percentage of responders aged 2 years is lower than 50% and increases steadily by age.

With respect to the present invention the term ‘polypeptide’ refers to an amino acid chain of any length, including a full-length protein, oligopeptides, short peptides and fragments thereof, wherein the amino acid residues are linked by covalent bonds.

“Isolated” and “purified”: The term “isolated” requires the material to be removed from the environment in which it was present originally. For example, a polypeptide or nucleic acid, which is expressed in a cell, is not isolated. However, the same polypeptide or nucleic acid, when separated from some or all of the coexisting material occurring in the original environment, will be considered as isolated. It is in accordance with this definition to regard polypeptides and nucleic acids present in cell lysates as isolated. By “purifying” a compound such as a polypeptide or a nucleic acid is meant increasing the degree of purity of a preparation of the compound by removing completely or partially at least one contaminant from the preparation. When applied to a preparation of a compound the term “degree of purity” refers to its relative content by weight of the compound of interest, based on the total weight of the preparation. The degree of purity of a compound may be within the range of 1-100%, such as from 1-100%, 10-100%, 20-100%, 30-100%, 40-100%, 50-100%, 300-100%, 70-100%, 80-100% and 90-100%. ‘Substantially pure’ is herein used to describe a polypeptide or a nucleic acid with a degree of purity of at least 70%, such as at least 75%, at least 80%, at least 85%, at least 90% at least 95%, at least 99% or preferably substantially pure from other components. The % value herein indicates % (w/w).

### FIGURE LEGENDS

FIG. 1

Quantitative fluorometric measurements (m1-3) of plasma Ab recognition of VSA expressed by P. falciparum isolates. Parasites (columns) were obtained from 68 pediatric patients from Ghana (parasite donors). Plasma samples (rows) were obtained from 96 healthy children from the same area as the patients. Small squares represent specific parasite/plasma combinations. For each such combination, Ab levels are indicated by the shading of the square (m1-4). The healthy plasma donors (rows) are sorted by age, and within each of the two clinical categories the parasite isolates (columns) are sorted according to the age of the parasite donors (malaria patients). Within parasite donor age groups, individual isolates are sorted according to level of VSA IgG recognition (sum of scores). Small numbers along the right and bottom edges are for enumeration of plasma samples and parasites, respectively.

FIG. 2

Age dependency of Ab recognition of VSA expressed by 68 P. falciparum isolates. For each parasite/plasma combination, the Ab recognition was scored on a six-level scale, according to Ab recognition of the isolate by 2-fold dilutions of a pool of plasma from adult, parasite-exposed Ghanaians. The overall Ab recognition of individual isolate was subsequently calculated as the sum of scores obtained with each of the 96 plasma samples. The dependency upon the age of the parasite donors (malaria patients) is shown in A and B, whereas the dependency upon the age of the healthy plasma donors is shown in C and D. Parasite isolates obtained from patients with severe P. falciparum malaria are shown in A and C, whereas parasites from patients with nonsevere malaria are shown in B and D. In all panels, means and 95% confidence intervals are indicated.

FIG. 3

Distribution of patient age (A) and VSA Ab fluorescence sum of scores (B) according to cluster assignment of the infecting P. falciparum isolate. Median (center line), 25th and 75th percentiles (box), 10th and 90th percentiles (vertical lines), and outliers (•) are shown for each cluster.

FIG. 4

Plasma Ab recognition of VSA expressed by parasite isolates obtained from 68 Ghanaian children with P. falciparum malaria. The level of fluorescence (mean and 95% confidence intervals) obtained with 2-fold dilutions of a plasma pool from parasite-exposed adult Ghanaians is shown, including the regression line (solid line) and its 95% confidence interval (dashed lines). A, Ab recognition of isolates according to their origin from patients with severe (•) or nonsevere (◯)P. falciparum malaria. B, Ab recognition of isolates according to their origin from young (•; 3-4 years of age) or older (◯; 5-11 years of age) P. falciparum malaria patients.

FIG. 5

Levels of IgG with specificity for variant surface antigens in P. falciparum 3D7 before (panels A-B) and after (C-D) in vitro selection by iRBC reactivity with IgG in the plasma of semi-immune Ghanaian children. Open histograms show VSA-specific IgG reactivity in plasma from semi-immune Ghanaian children (A, C) or clinically immune Ghanaian adults (B, D). Shaded histograms show corresponding reactivity in plasma from donors without P. falciparum exposure (negative control).

FIG. 6

An in vitro culture of ethidium bromide-labelled P. falciparum 3D7-infected RBC following sequential exposure to the SM1 plasma pool, biotinylated secondary anti-human IgG, and streptavidin-coated DynaBeads (m3-1, m3-2). Uninfected RBC (white left-arrows), and iRBC coated (white down-arrows) or not coated (white up-arrows) with DynaBeads, as well as free DynaBeads (black arrows) can be seen in the micrograph.

FIG. 7

Plasma levels of IgG with specificity for variant surface antigens expressed by 3D7, 3D7_{SM1}, and five P. falciparum field isolates from Sudan. Plasma was obtained from Sudanese (A) and Tanzanian (B) individuals, and levels are given on a semi-quantitative scale determined by recognition of intact iRBC by IgG in plasma pools from non-exposed donors (negative control pool) and highly exposed donors (positive control pool), respectively. IgG reactivity in plasma from the five parasite donors to the autologous P. falciparum isolates are indicated by heavy lines.

FIG. 8

Adhesion of 3D7- and 3D7_{SM1}-infected RBC to wild-type (CHO-0) and CD36-transfected (CHO-CD36) Chinese hamster ovary (CHO) cells (m4-1).

FIG. 9

A) Schematic presentation of all 3D7 var gene sequence analyses (m2-1, m2-2, m2-3). Gene names, chromosomal location, transcriptional direction and domain structure are shown along with the cluster to which each gene was assigned by the sequences analyses. Sequences that could not be assigned to any cluster were named X. Three major var gene groups (group A-C), two intermediate groups group B/A and group B/C and two unique genes representing var1 and var2 var gene families were defined (framed). B) Sequence analyses of var genes from other P. falciparum strains than 3D7. Protein accession numbers, originating strain, domain structure and the closest related 3D7 var 5′ sequence are shown along with sequence group allocations as defined in 3D7. *) The genes were assigned to group A, as their DBL1α sequences clustered together with other group A sequences in analysis of DBLα sequences. **) Pseudogene, belongs to the var1 family ̂) Upstream sequences with atypically low similarity to upsB or upsC sequences.

FIG. 10

Schematic representation of head-to-head genomic organisation of rif and upsA flanked var genes. Nine genes are flanked by a rif gene, which has its initiation codon approximately 3 or 4 kb upstream from the var initiation codon, and one var gene by another var gene at −2 kb. Punctured lines represent upsA, dotted lines upsA-rif and full line upsBsh. The diamond marks the putative termination site of upsA characterised by a stretch of TA repeats. Sizes of genes are not in scale.

FIG. 11

Distance tree of 3D7 var gene 500 bp 3′ region generated using the p-distance/NJ method (m2-3). The four dense clusters A through D were supported by both bootstrapping and maximum parsimony (MP) tree (not shown). The relationship of the remaining sequences could not be verified by the MP tree making method. Numbers at the nodes represent bootstrap proportions (BP) on 1000 replicates. The scale bar represents the proportion of different nucleotide compared. PlasmoDB accession numbers are shown. Genes with assigned cluster are collected in FIG. 9.

FIG. 12

Distance tree of DBLαCIDR1 domains of 3D7 PfEMP1 and pseudogene PFE1640w generated using the p-distance/NJ method (m2-3). The clusters A through E were supported by both bootstrapping and maximum parsimony tree (not shown). Numbers at the nodes represent bootstrap proportions (BP) on 1000 replicates. The scale bar represents the proportion of different amino acids compared. PlasmoDB accession numbers are shown.

FIG. 13

Fold changes in var gene transcription by late-stage P. falciparum 3D7 before and after antibody-selection of parasites for selection of VSA_{SM}-type antigens using DynaBeads coated with plasma IgG from three different pools of plasma from semi-immune African children (SM1, SM2, SM3) (m3-2). Transcription levels were measured using real-time PCR and primers specific for 59 var genes and one pseudogene (Salanti et al., 2003) (m5-2). Panel A shows mean fold changes (±SD; 5 experiments) in var gene transcription related to selection using the SM1 pool. Panel B shows fold change values in one experiment using the SM2 pool, while Panel C summarizes one experiment using the SM3 pool. Black bars indicate the 15 most highly transcribed var genes in selected and/or unselected 3D7. A 3-fold change in var gene transcription (dashed lines) was arbitrarily defined as the cut-off for biologically significant changes in var gene transcription. Primers see Table 1. Grouping of var genes is as described in (Lavstsen et al., 2003).

FIG. 14

Changes in var gene transcription in synchronised ring-stage (30 hr) Plasmodium falciparum 3D7 in vitro selected with a pool of plasma antibodies from children from semi-immune African children (SM1, SM2) (se legend to FIG. 13). Panel A shows mean fold changes (±SD; 5 experiments) in var gene transcription related to selection using the SM1 pool. Panel B shows fold change values in one experiment using the SM2 pool. A fold change of 2 (dotted lines) was defined as the cut-off for biological interesting changes in var gene transcription.

FIG. 15

Relative levels of var transcripts in 10 μg of total RNA obtained from 3D7 (lane 1) and antibody-selected 3D7 (lane 2) ring stage iRBC. Northern blots (m7-1) probed with DIG labelled RNA probes targeting (Panel A) PFD1235w/MAL7P1.1 (SEQ ID NO.: 1). Transcript size is 12.5 kb. (Panel B) Ethidium bromide-stained gel shown to allow comparison of total RNA amounts.

FIG. 16

Expression of PfEMP1 in 3D7 and antibody-selected 3D7 trophozoite/schizont stage iRBC. Western blots (m10-5, m10-6) of SDS-extracts obtained before (Lane A) and following (Lane B) antibody selection. Samples were run in 5% SDS-PAGE gels, blotted and probed with antibodies to a relatively conserved sequence of the intracellular acidic segment ATS/Exon2 (lanes A-B) and a specific antibody raised against DBL5-8 of VAR4 (SEQ ID NO.: 2)(Lanes C-D). The antibody-selected parasite expressed the product of PFD1235w/MAL7P1.1 (***) (SEQ ID NO.: 2) and a PfEMP1 with molecular weight (MW) corresponding to the predicted MW of MAL6P1.316 (**). Both selected and unselected parasites expressed a PfEMP1 of a MW corresponding to the predicted MW of PF08_{—}0107 (*). SDS-extracts of RBC infected by NF54 obtained from a Dutch volunteer (Hermsen et al., 2001). Extracts were obtained on day 8, 9, and 10 (Lanes E-G) and the blot probed with the DBL5-δ antibody used in Lanes C-D.

FIG. 17

Surface expression of antigens in P. falciparum 3D7 before (A1-5) and after (B1-5) antibody selection (m3-2, m10-6). Murine plasma antibodies against recombinant DBL5-δ of VAR4 (SEQ ID NO.: 2) reacted with the surface of most antibody-selected 3D7 iRBC in FACS (B1; green line) and fluorescence microscopy (B2; green dots). The antibodies only reacted with a minority of unselected 3D7 (A1, A2), and with none of the trypsin-treated iRBC from antibody-selected 3D7 (B1; purple line). Shaded histograms show flow cytometry reactivity in pre-vaccination mouse plasma. VSA-specific IgG (A3, B3) reactivity in plasma from a semi-immune African child (red line) and a clinically immune African adult (blue line) confirming the VSA_{UM} phenotype of unselected 3D7 and the VSA_{SM} phenotype of antibody-selected 3D7. Localization of the VAR4 (SEQ ID NO.: 2) using confocal microscopy and murine plasma anti-DBL5-δ antibodies (A4, A5, B4, B5). Ethidium-bromide staining of DNA in the nuclei is red/orange and staining of VAR4 (SEQ ID NO.: 2) using FITC-labeled antibodies is green. Pre-vaccination mouse plasma did not stain iRBC (data not shown).

FIG. 18

Plasma antibody levels to recombinant DBL5-δ (A), CIDR1-α (B), DBL3-β (C), and NTS (D) domains of the VAR4 (SEQ ID NO.: 2) in Tanzanian children and adults, and in Danish donors without P. falciparum exposure (DK) (m12-1). For competition ELISA experiments (m12-2), plates were coated with recombinant CIDR1-α domains of the proteins encoded by PFD1235w/MAL7P1.1 (SEQ ID NO.: 1 and SEQ ID NO.: 2) (E) and PFE1644w/var1 (F), and ELISA reactivity measured in three different plasma samples, which had been pre-incubated with increasing concentrations of homologous or heterologous fusion protein as indicated in the top part of each panel. Non-depleted (ND) plasma was included for comparison.

FIG. 19

Similarity of the PFD1235w/MAL7P1.1 encoded protein (SEQ ID NO.: 2) homologues in genotypically distinct parasite isolates. Alignment of DBL1-α/CIDR1-α from two peripheral blood parasites from children (BM021 and BM48) and the protein product of PFD1235w/MAL7P1.1 (SEQ ID NO.: 2) from 3D7 covering amino acids 842-1602 in 3D7 SEQ ID NO. 2. Identical residues appear on black background, conserved amino acid changes on grey, and radical changes on white background.

FIG. 20

Fold changes in var gene transcription in ring-stage (30 hr) (Panel A) and by late-stage (Panel B) P. falciparum 3D7 before and after in vitro selection on human endothelial cells for selection of parasites expressing VSA_{SM}-type antigens (m6-1, m6-2). Transcription levels were measured using real-time PCR and primers specific for 59 var genes and one pseudogene (Salanti et al., 2003). The two panels shows fold change values in one experiment. Black bars indicate the 15 most highly transcribed var genes in selected and/or unselected 3D7. A 3-fold change in var gene transcription (dashed lines) was arbitrarily defined as the cut-off for biologically significant changes in var gene transcription. Primers see Table 1. Grouping of var genes is as described in (Lavstsen et al., 2003).

### EXAMPLES

### Example 1

Erythrocytes infected by P. Falciparum parasites causing severe malaria (SM) are stronger and more commonly recognised by igG in plasma of malaria-exposed individuals than erythrocytes infected by other P. falciparum parasites

P. falciparum-infected red blood cells (iRBC) adhere to endothelial host receptors through parasite-encoded, clonally variant surface antigens (VSA). The VSA-mediated iRBC adhesion and the acquired VSA-specific antibody response is linked to disease severity. Parasites isolated from young children with severe malaria (SM) express a limited and conserved set of VSA (VSA_{SM}) that are both stronger and more commonly recognised by IgG in the plasma of malaria-exposed individuals than VSA (VSA_{UM}) expressed by parasites causing uncomplicated malaria (UM) in older semi-immune children. It is therefore likely that the SM-specific protective immunity acquired in young children in areas of intense parasite transmission is based on antibodies to VSA_{SM} that inhibits the adhesion to endothelial cells of parasites expressing VSA_{SM} on the surface of infected erythrocytes.

### Materials and Methods

m1-1. Isolation of iRBC from malaria patients: Circulating human erythrocytes infected with Plasmodium falciparum (iRBC) were collected in vacutainers containing either heparin or citrate-phosphate-dextrose (CPD) as anticoagulant. Plasma and white blood cells were removed upon centrifugation at 800×g, and the erythrocyte pellet resuspended in an equal volume of freezing solution (28% (v/v) glycerol in 4.2% (w/v) sorbitol and 0.9% (w/v) NaCl in H₂O) and snap-frozen in liquid Nitrogen.
   In vitro culture of P. falciparum parasites: Cryopreserved iRBC were restored by thawing at 37° C. followed by washing in 3.5% NaCl₂ and washing twice in RPMI 1640 culture medium (http://www.lifetech.com). Parasites were maintained in a 5% suspension culture of uninfected human O⁺ erythrocytes in RPMI 1640, supplemented with Albumax, hypoxanthin, glutamine, gentamycin (all http://www.lifetech.com), and non-immune human serum. Culture medium was changed and Giemsa-stained smears were prepared for microscopy on a daily basis.
m1-2. Purification of iRBC from cultures: iRBC with haemozoin-containing trophozoites and schizonts were purified from in vitro cultures (m1-1) by magnet-activated cell sorting (MACS; http://www.miltenvibiotec.com), exploiting the magnetic properties of haemozoin. In short, iRBC were passed through a size-C MACS column mounted with a 0.9 mm×40 mm needle. The column was washed with phosphate buffered saline (PBS) supplemented with 2% foetal calf serum (FCS; PBS-S) until no erythrocytes could be seen in the eluate. The column was removed from the magnet, and the trophozoite- and schizont-containing iRBC retained in the column were then released by further washing. A purity of trophozoite-/schizont-infected iRBC >90% was usually reached by this procedure.
m1-3. Detection of human VSA-specific IgG: Purified IRBC (m1-2) were labelled with 1 μl ethidium bromide (EB; http://www.sigma-aldrich.com) solution (0.1 mg/ml) per 10⁵ erythrocytes to allow discrimination between nucleic acid-containing iRBC and uninfected erythrocytes devoid of DNA/RNA. For each sample, 2×10⁵ erythrocytes in 100 μl PBS-S (m1-3) were used. EB-labelled iRBC were mixed with 1-5 μl of human plasma or antibody preparation, followed by goat anti-human IgG (http://www.dako.com), diluted 1:200 and by fluorescein isothiocyanate (FITC)-conjugated rabbit anti-goat Ig (http://www.dako.com), diluted 1:25. The antibodies were diluted in PBS-S, and 100 μL of the dilution was added per sample. At each step, samples were incubated for 30 min at 5° C. The samples were washed twice in 3 ml PBS-S between each incubation step and once after the last. Samples were kept overnight at 5° C. before analysis on a Coulter EPICS XL-MCL flow cytometer (http://beckman.com).
m1-4. For quantification of FITC fluorescence, the mean fluorescence intensity (MFI) of the ethidium bromide positive red blood cells was calculated using WinList software (http://www.vsh.com). Plasma from Danish donors never exposed to falciparum malaria did not label uninfected erythrocytes or iRBC above the level of the secondary and tertiary antibodies alone. In contrast, the plasma pool prepared from hyper-immune Ghanaians selectively labelled iRBC but not uninfected erythrocytes.
m1-5. Human plasma samples tested: The individual human plasma samples were obtained from the following groups of individuals:
   Plasma from Danish adults without exposure to P. falciparum parasites were obtained at the Copenhagen University Hospital (Rigshospitalet) from laboratory staff and blood donors being screened for the presence of anti-RhD antibodies.
   Plasma from 96 Ghanaian children living in Dodowa Town, 50 km northeast of Accra. The area is characterized by hyperendemic, seasonal transmission of P. falciparum parasites. All children were healthy at the time of blood sampling.
   A pool of plasma from healthy, parasite-exposed adults from the village of Gomoa Onyadze, 80 km west of Accra (Nielsen et al. 2002).

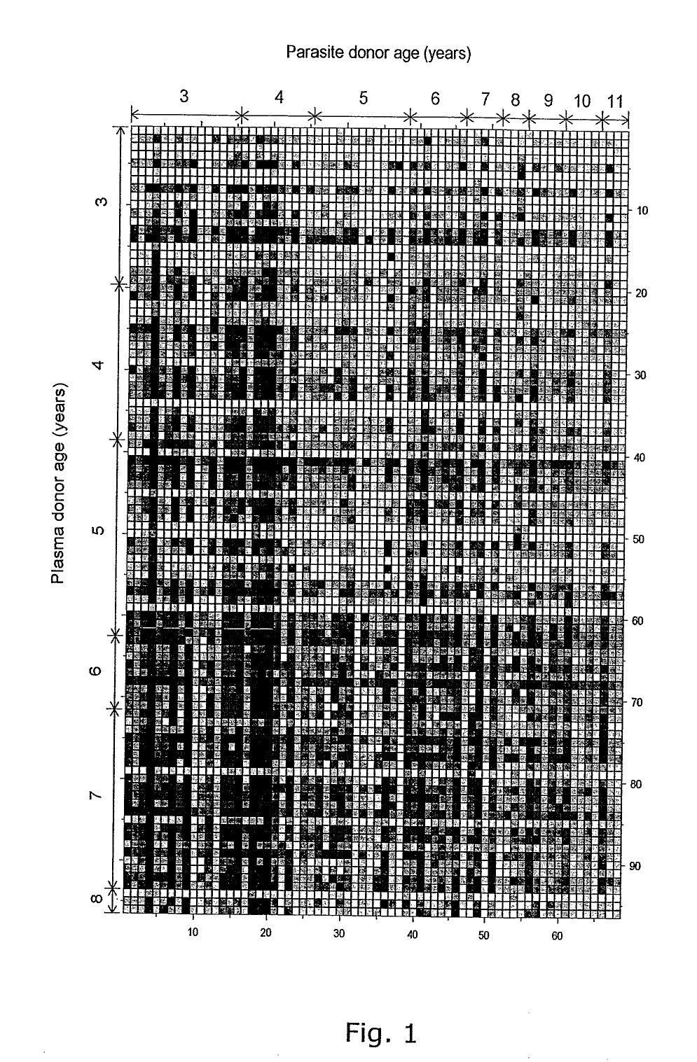

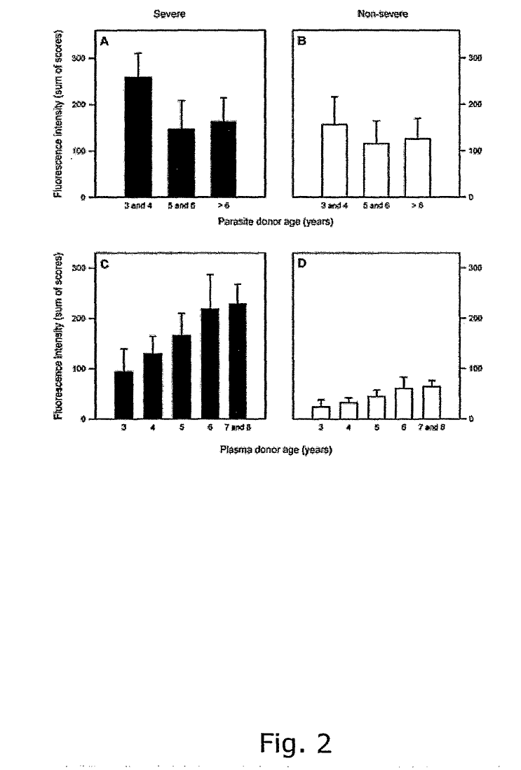

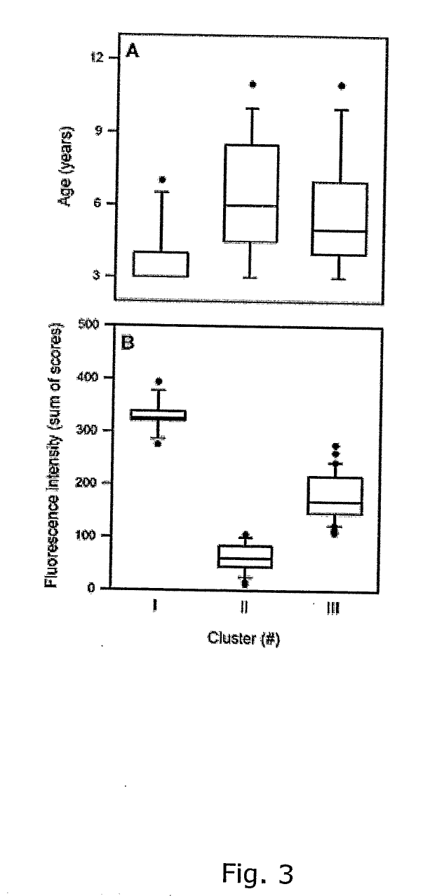

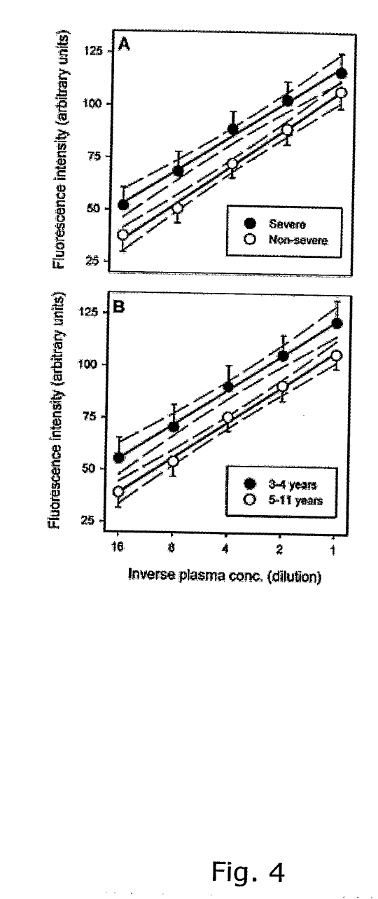

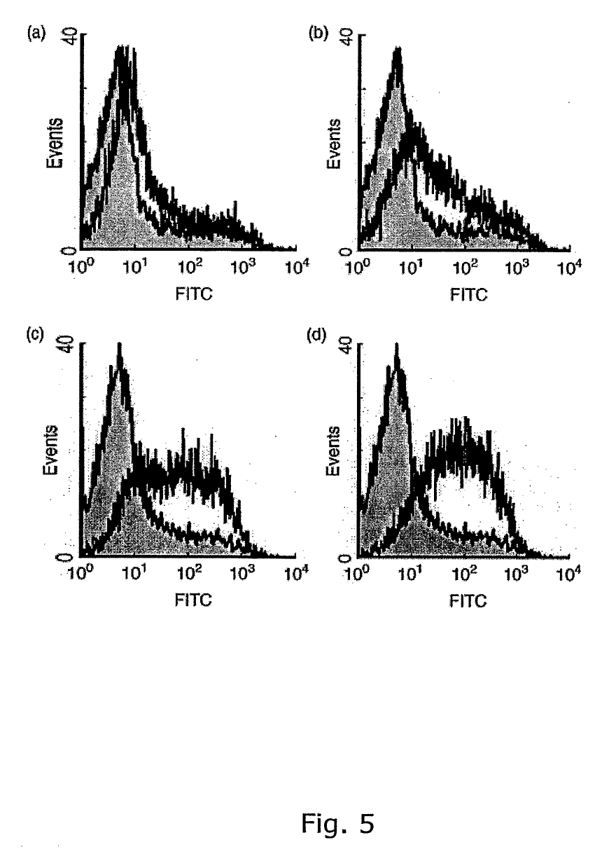

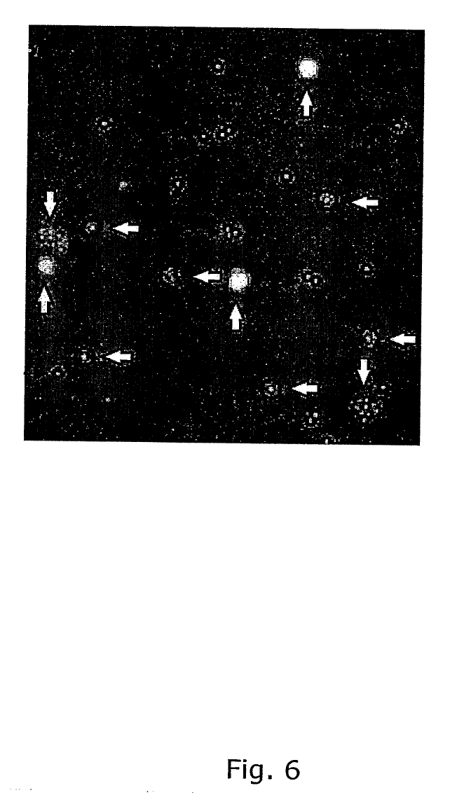

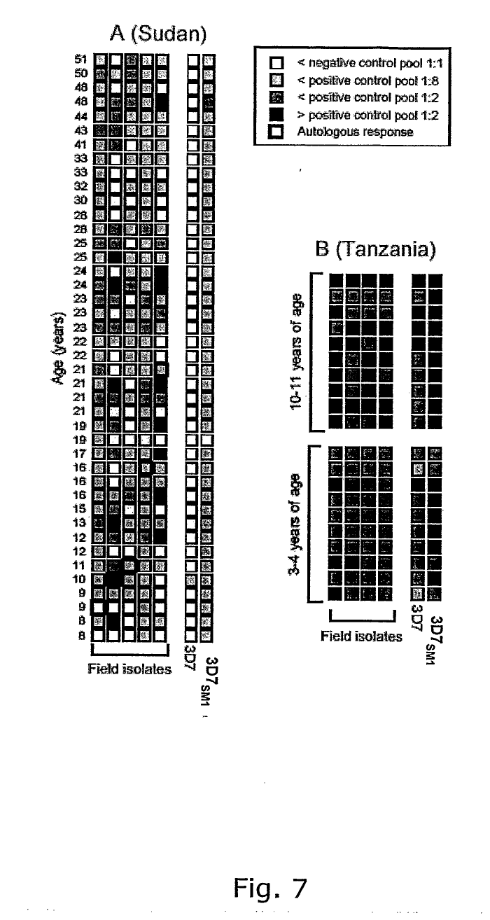

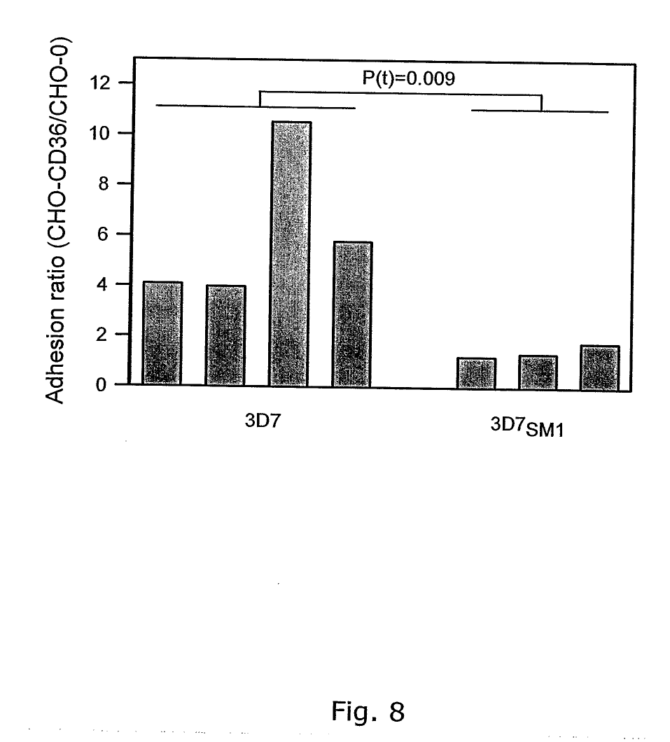

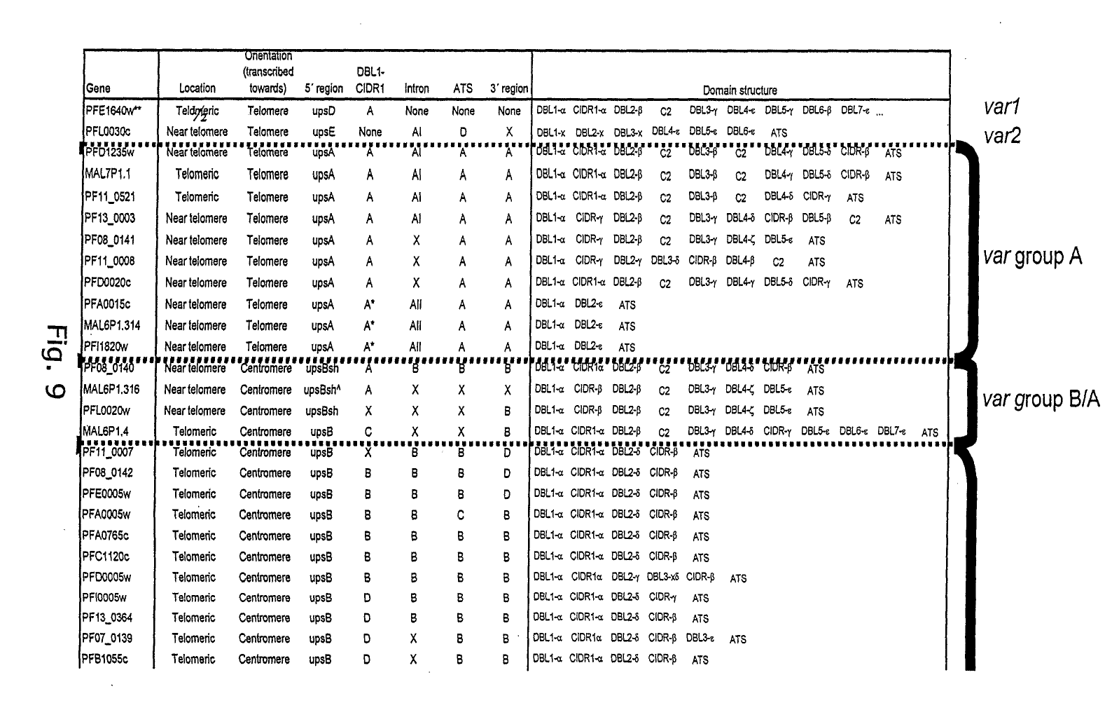

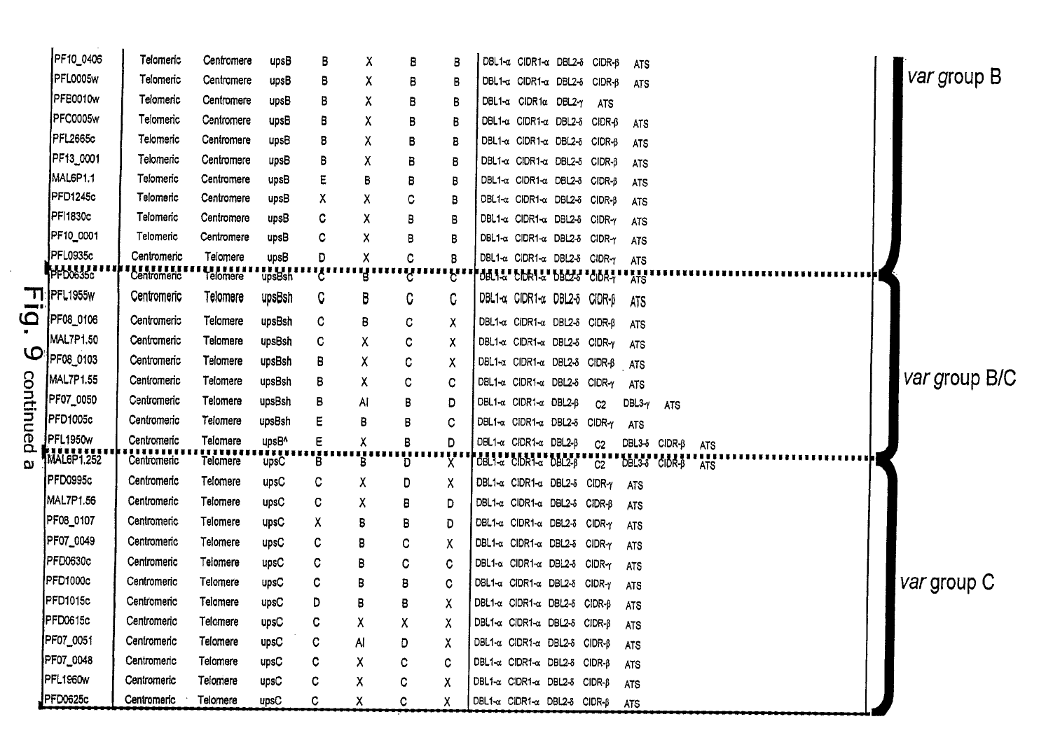

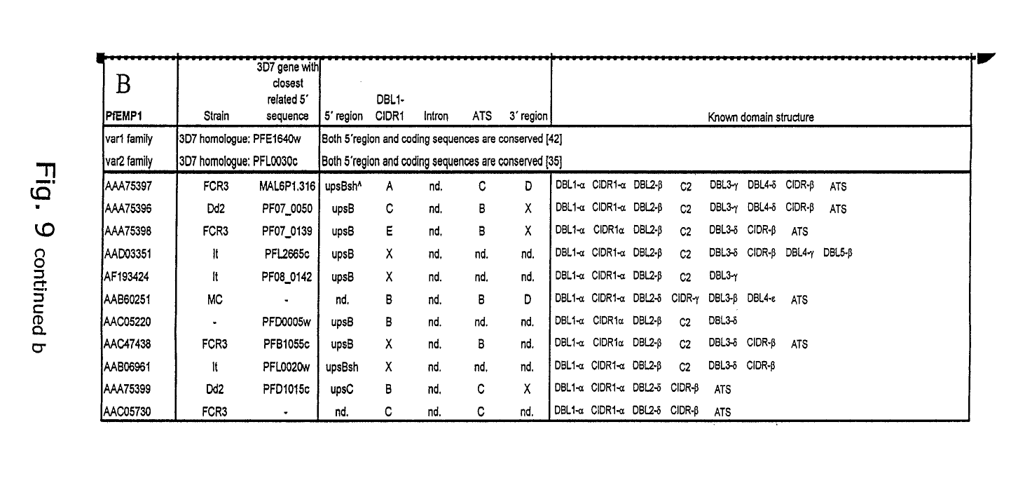

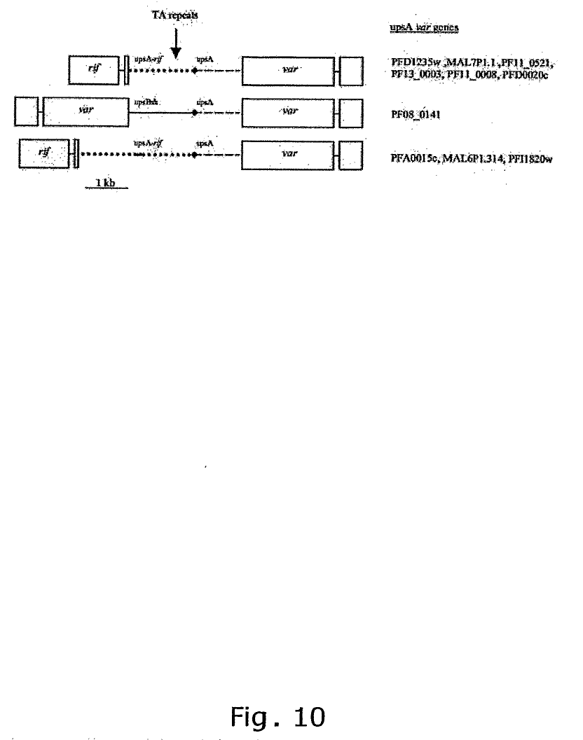

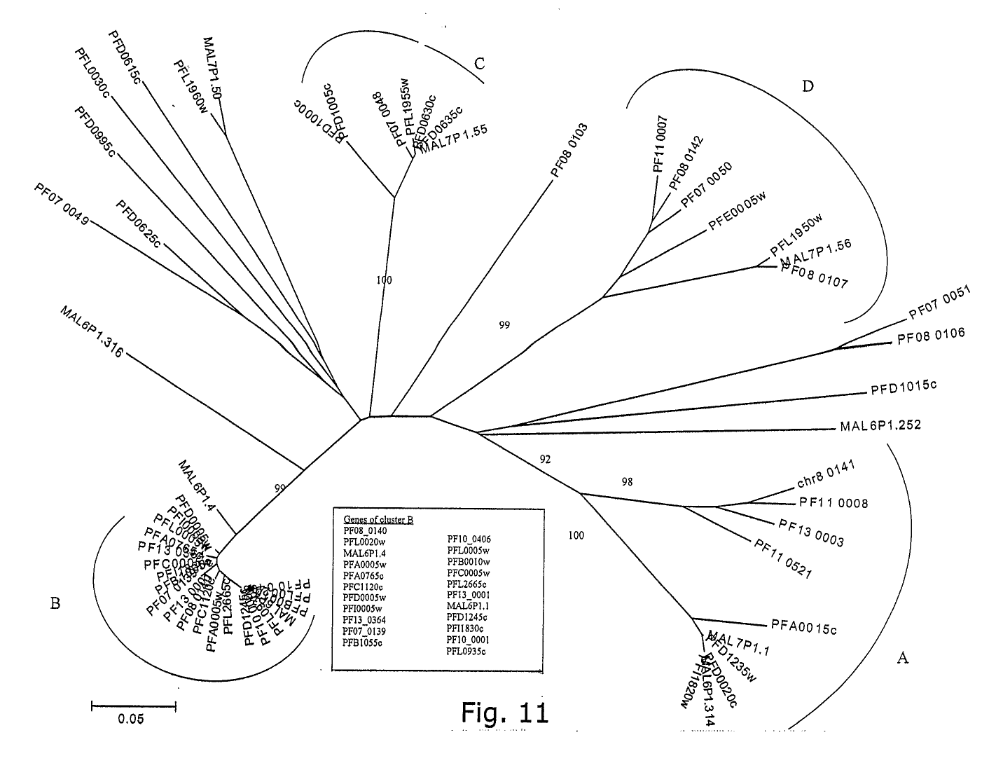

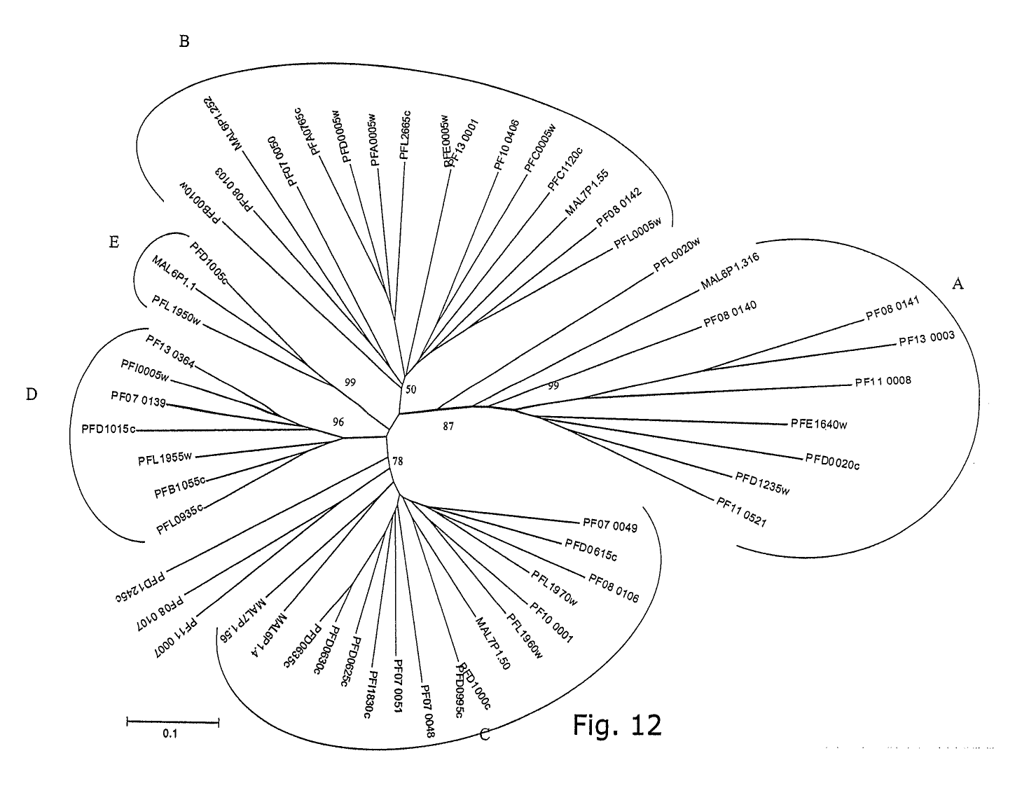

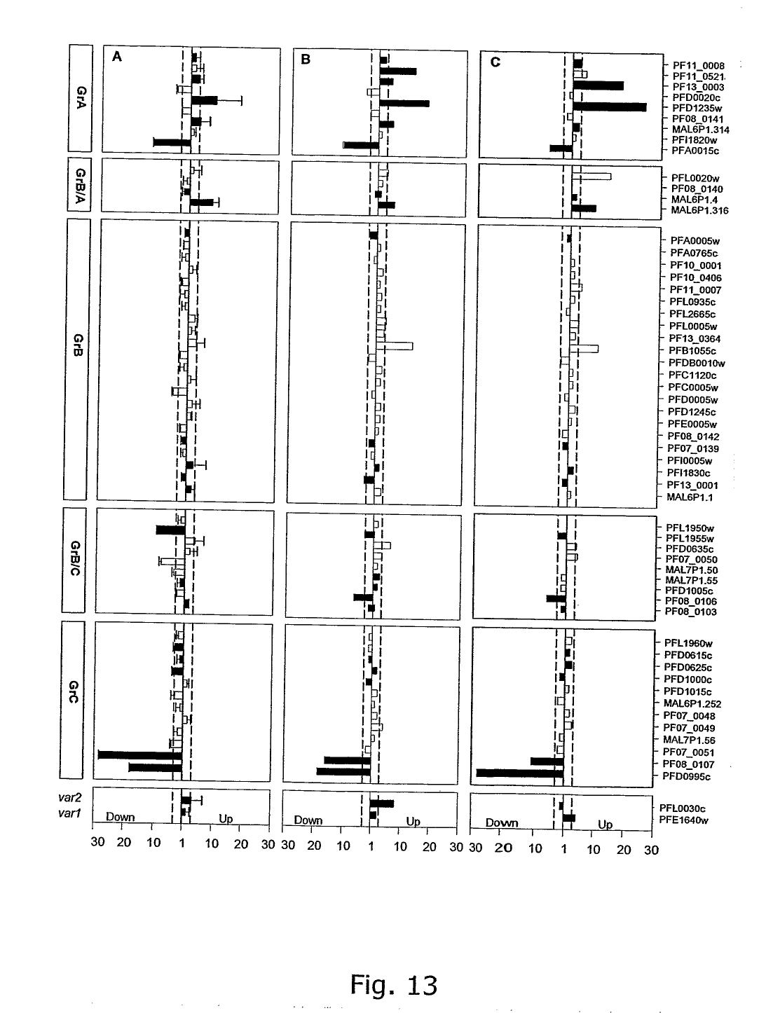

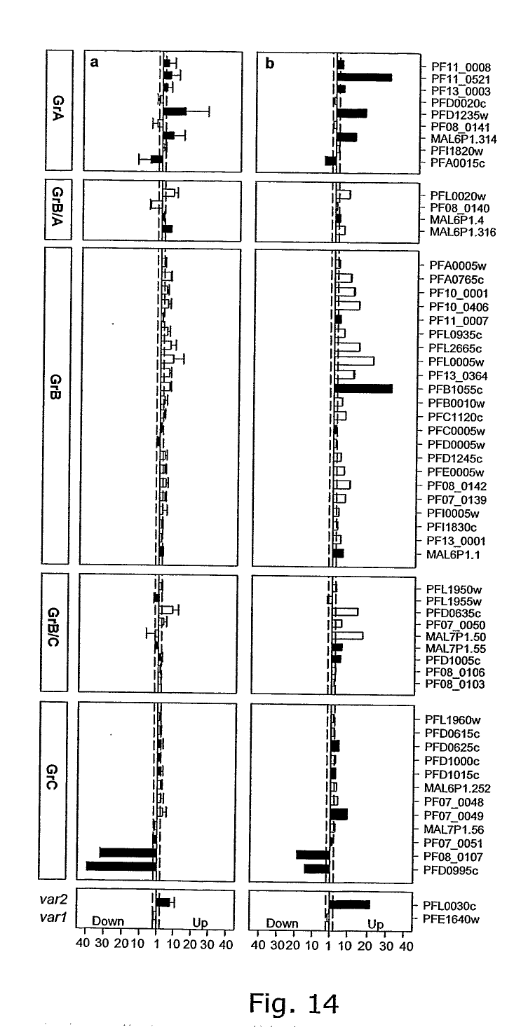

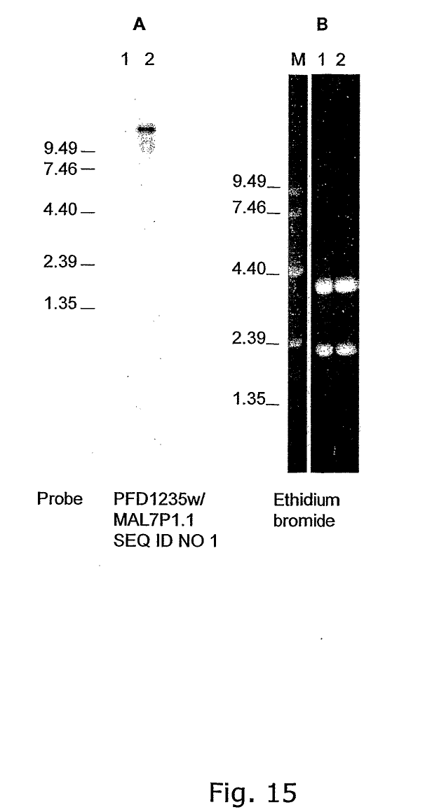

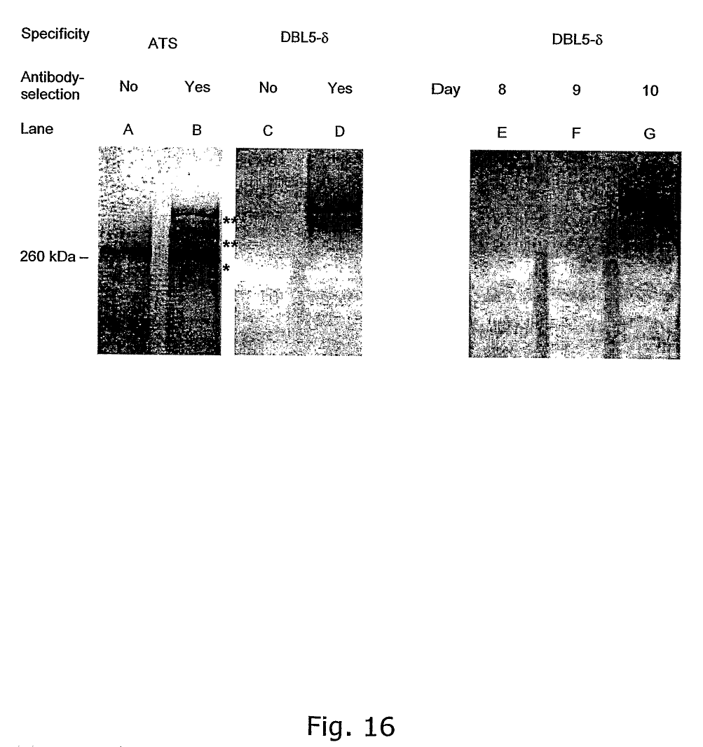

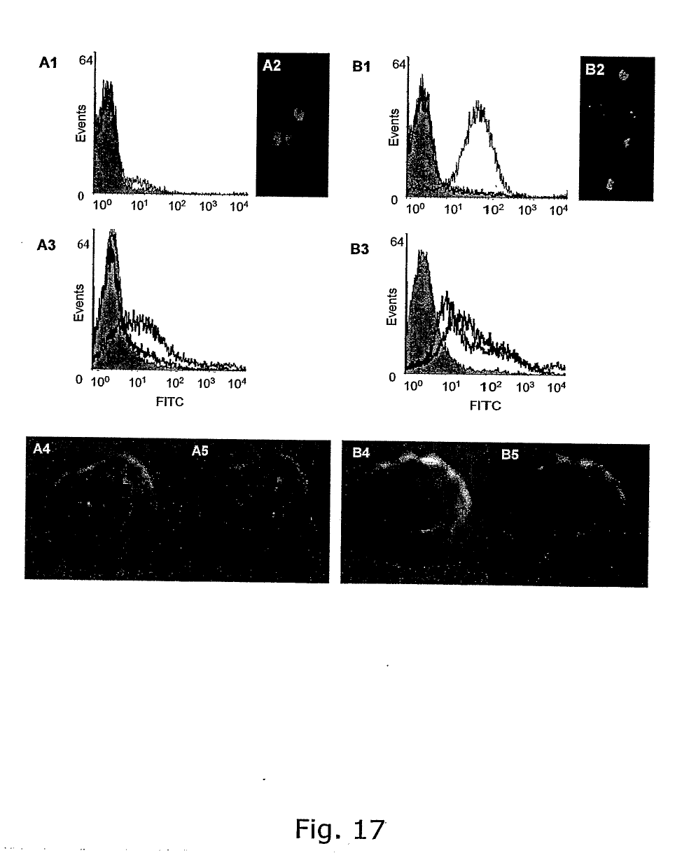

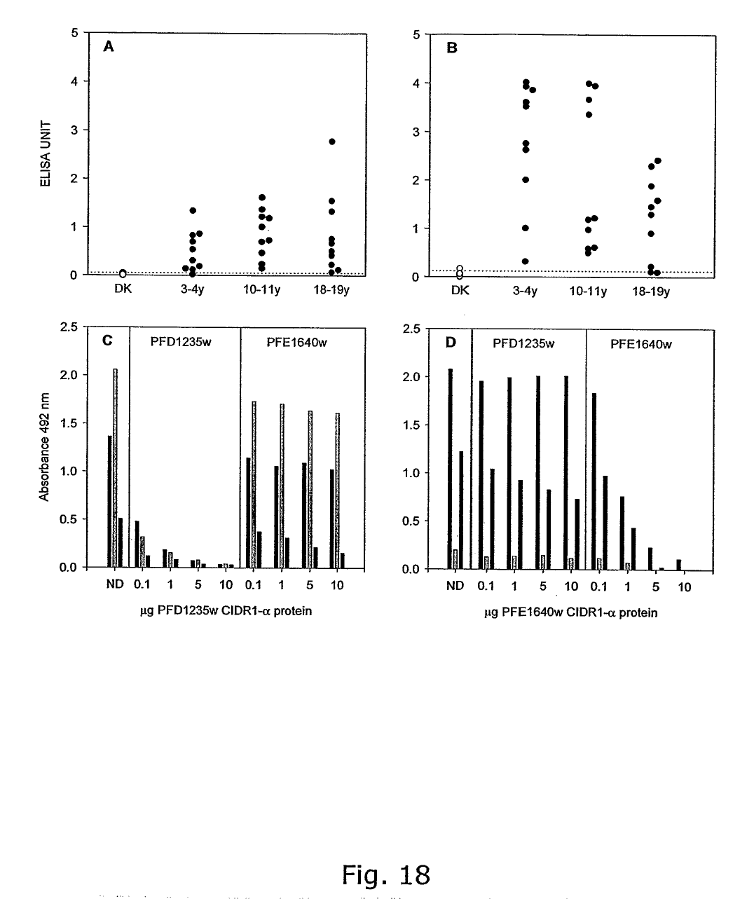

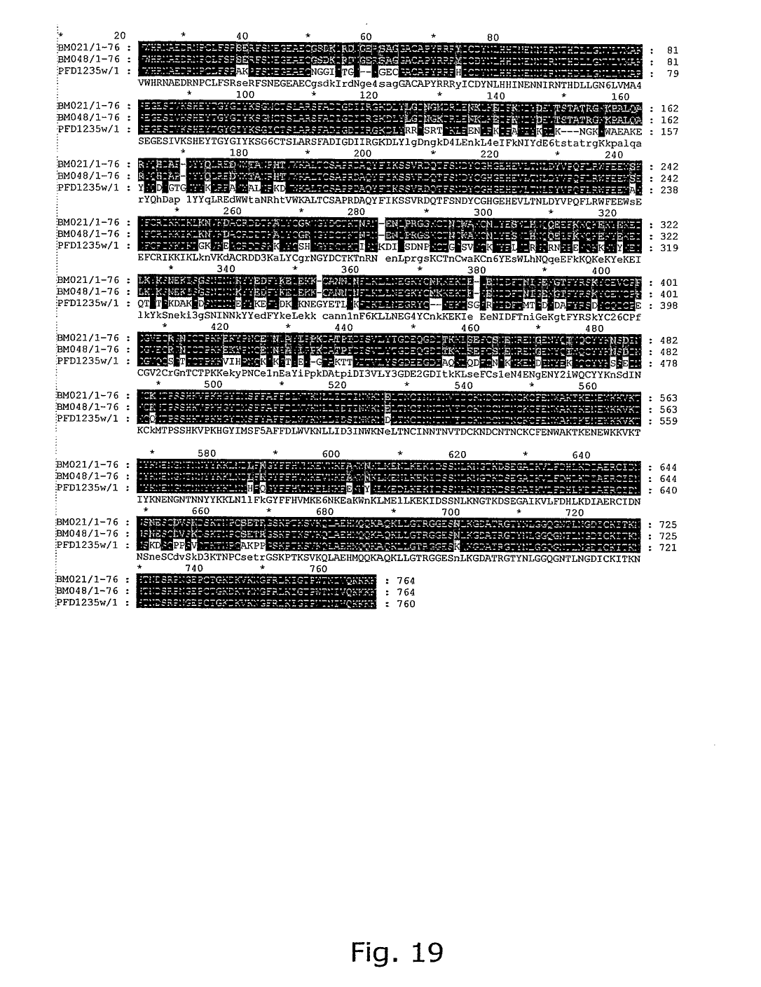

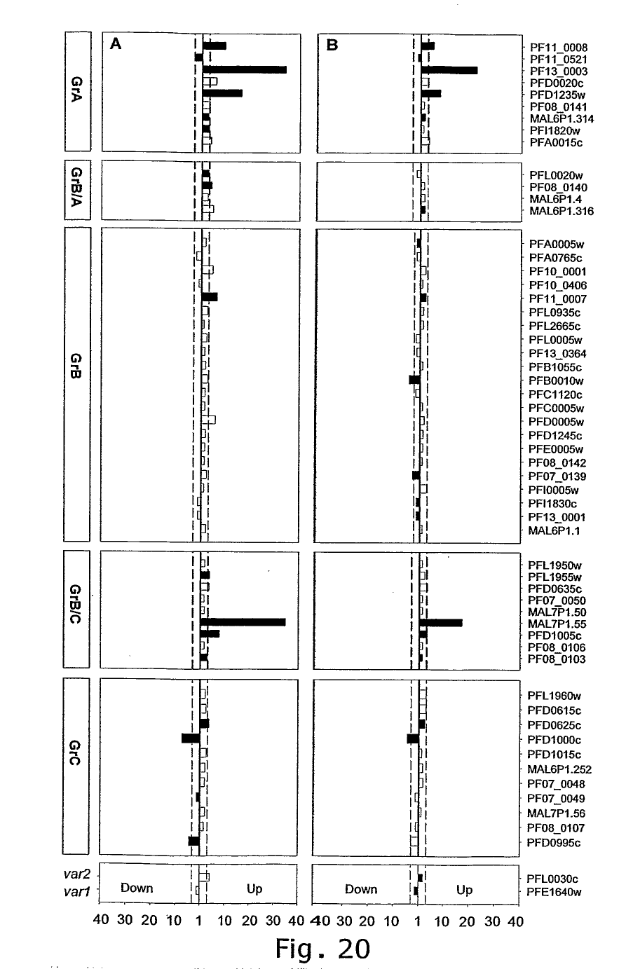

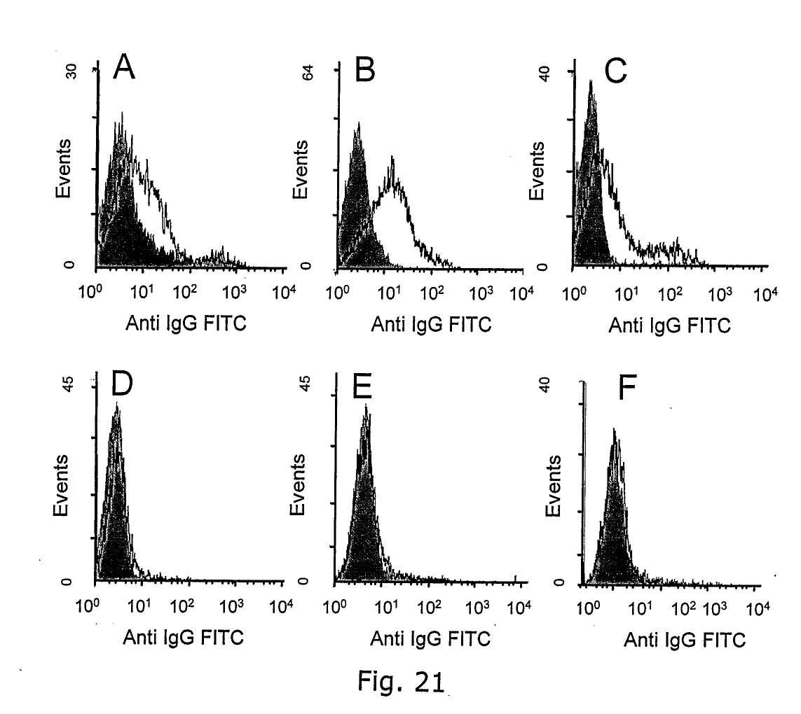

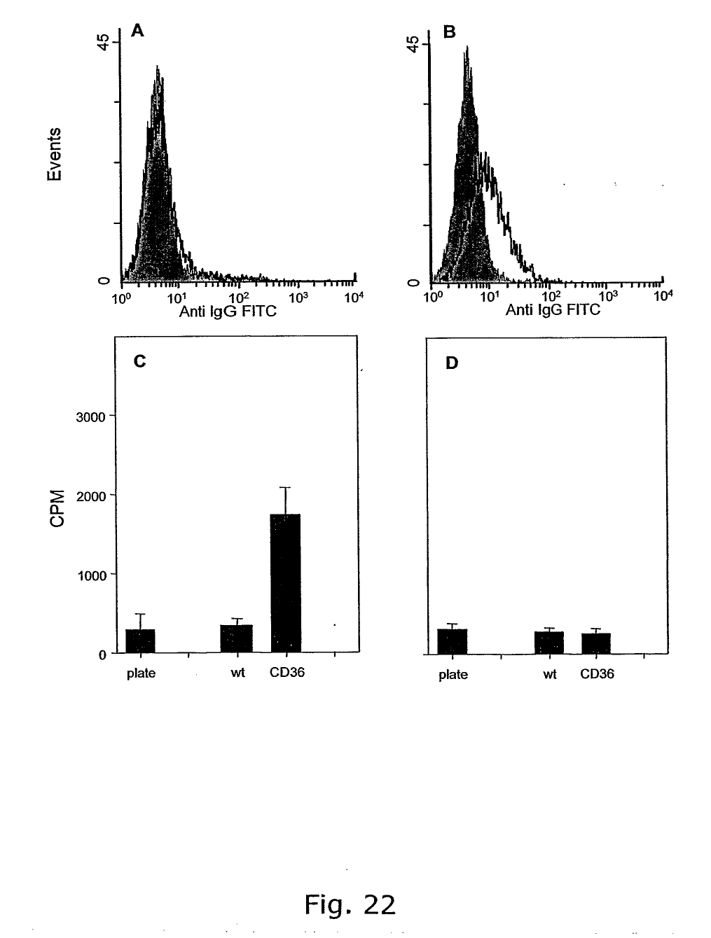

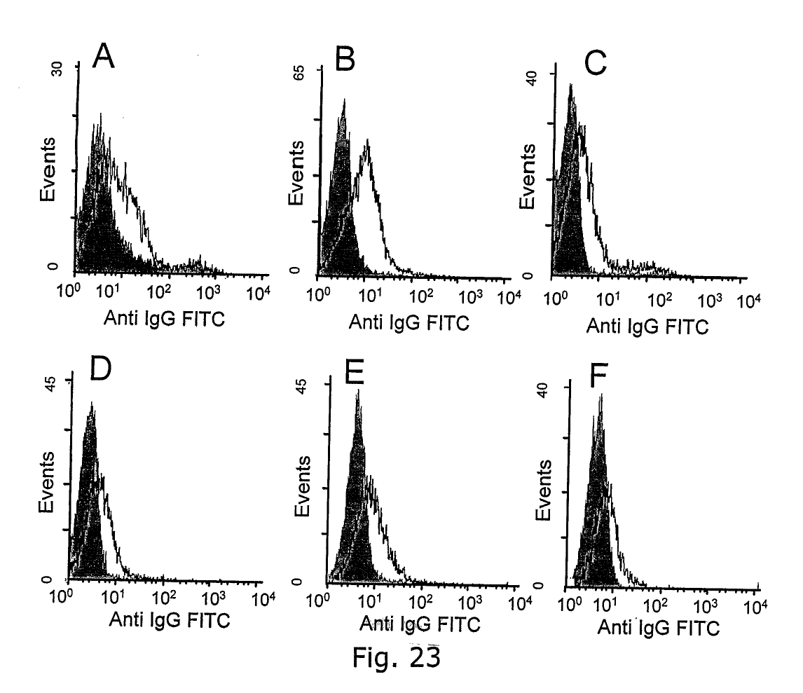

To corroborate the above hypothesis levels of antibodies in plasma from 96 healthy children, aged 3-8 years, with specificity for each of the 68 parasite isolates (FIG. 1) were measured (m1-4). Overall, antibody (Ab) recognition of the parasite VSA differed widely among plasma donors. While plasma samples from some children contained barely detectable levels of Abs specific for VSA expressed by any of the isolates (e.g., plasma donors 15, 34, and 59; see FIG. 1), others had high levels and a broad range of VSA-specific Ab (e.g., plasma donors 14, 41, and 84; FIG. 1). By analyzing the parasite-specific sum of scores from the 96×68 recognition matrix, we found that recognition of VSA was independently associated with both the age of the malaria patient (3-4, 5-30, and 7-11 years; p=0.005) and the clinical picture (severe or nonsevere; p=0.006, by two-factor ANOVA). There was no significant interaction between these two sources of variation (p=0.26). Pair wise multiple comparison procedures (Tukey's post-hoc test) showed that while the VSA sum of scores of parasites from the youngest patient group was significantly different from that from either of the two other age groups (p<0.05), the latter two were not significantly different from each other (p≧0.05). The relationship between severity and age of the parasite donor is illustrated in FIG. 2, A and B. These results show that Ab recognition of parasite VSA was independently affected by both the age and the clinical severity of the malaria patient from whom the parasite was obtained. This is important, because disease severity is inversely correlated with age in areas of endemic parasite transmission, which in all likelihood reflects age-dependent acquisition of protective immunity (reviewed in Riley et al. 1994). Protective immunity appears to involve acquisition of Ab responses to a broad range of VSA (Bull et al. 1998), and consistent with this observation we found that VSA Ab levels correlated with the age of the healthy plasma donors (FIG. 2, C and D). The authenticity of our finding of independent effects of patient age and disease severity is supported by the fact that the age distributions of children with severe and nonsevere disease were similar (by t test, p=0.5) in the present study (5.30±0.4 and 30.0±0.5 years, respectively; mean±SD), due to the exclusion of children <3 years of age.

To further substantiate our findings and to investigate whether parasites from patients with severe P. falciparum malaria expressed particular VSA, we used the 96×68 recognition matrix (FIG. 1) to search for patterns of similarity in the VSA Ab recognition of the parasite isolates. Hierarchical cluster analysis identified three main clusters (data not shown). In one of these (cluster I), all but one (90%) of the isolates were from severe cases, whereas this was the case for only 5 of 20 (25%) in cluster II. The third and largest cluster (III) showed an intermediate pattern, with 21 of 38 (55%) isolates from patients with severe malaria (FIG. 3). The proportion of isolates from severe patients in the three clusters was thus quite different (by X² test, p=0.009). When we analyzed the age distribution of the patients donating the parasites within the clusters, cluster I was composed of parasites from young patients, whereas the other two clusters contained parasites from older patients (FIG. 3A). The cluster-specific differences in patient age composition did not quite reach conventional statistical significance (by Kruskal-Wallis test, p=0.06). The distribution of the parasite-specific sum of scores among the clusters showed that cluster I was composed entirely of parasites expressing very well-recognized VSA, whereas the opposite was true for cluster II. Again, cluster III formed an intermediate group (FIG. 3B). The distribution of sum of scores was significantly different among the three clusters (by Kruskal-Wallis test, p<0.001), with all pairwise differences being significant (by Dunn's post-hoc test, p<0.01 in all cases).

We have used flow cytometry to measure VSA-specific IgG to provide evidence of modulation of VSA expression by acquired immunity. Our method is particularly suited to this type of analysis, as it allows unbiased and quantitative analysis of large matrixes of VSA and corresponding Abs of specified isotype. We found that the level of plasma IgG recognition of VSA expressed by P. falciparum isolates obtained from patients with severe malaria was approximately twice that of VSA from nonsevere isolates (FIG. 4A). In a similar way we found that VSA Ab recognition of isolates from young patients (3-4 years of age) was −2-fold that of isolates from older patients (5-11 years of age; FIG. 4B).

Thus, the VSA antibody recognition of parasites from severe patients is broader and more intense than recognition of VSA expressed by parasites from other malaria patients (FIGS. 1 and 2), independently of the age of the patient. Thus, our data suggest that acquisition of VSA-specific Ab responses gradually restricts the repertoire of VSA thatare compatible with parasite survival in the semi-immune host. Furthermore, it appears to limit the risk of severe disease by preventing the expression of VSA likely to cause life-threatening complications, such as cerebral malaria and severe anemia.

### Example 2

### Sub-Grouping of Plasmodium falciparum 3D7 var Genes Based on Sequence Analysis of Coding and Non-Coding Regions

PfEMP1 is a polymorphic family of high molecular weight adhesion antigens expressed on the surface of infected erythrocytes. PfEMP1 is an important target for protective immunity and is implicated in the pathology of malaria through its ability to adhere to host endothelial receptors. The accumulation of antibodies against a broad repertoire of PfEMP1s is probably the functional basis for the natural acquisition of immunity to malaria (Bull et al. 1998) All var genes are characterised by a two-exon structure. Exon 1 encodes a large extra-erythrocytic and highly variable region containing two to seven Duffy-binding like (DBL) domains and mostly one or two cysteine-rich inter-domain region (CIDR) domains. Based on sequence homologies, the DBL domains can be sub-divided into α, β, γ, δ, and ε types and the CIDR domains into CIDRα other (CIDR-O) types. A subset of var genes furthermore contains a second cysteine-rich domain called C2. Exon 2 encodes the intra-erythrocytic (cytoplasmic) and conserved part of the protein.

The entire genome of the P. falciparum clone 3D7 genome is now known, including its complete var gene repertoire. FIG. 9 shows the domain structure of each of the 59 var genes as well as the truncated pseudo-gene PFE1640w.

### Methods

m2-1. Nucleotide and deduced amino acid sequences as well as location and transcriptional directions of 3D7 var and rif genes were obtained from the Plasmodium Genome Resource—http://www.plasmodb.org.
m2-2. Alignments were performed using the ClustalW multiple alignment method, European Molecular Biology Laboratory, Heidelberg, Germany at default parameters (Gap Open: 10.00; Gap Extention: 0.20, Gap Separation Distance: 4, Protein weight matrix: Gonnet, DNA weight matrix: identity (IUB)). Alignments were corrected by hand using Bioedit (Hall, 1999) to assure homologous sequences for sequence analysis and tree-building. For distance tree-building, the var gene 5′ flanking regions were defined as the 600 bp, 1.4 kb or 2.0 kb upstream of the translation initiation codon and the 3′ flanking region as the 500 bp downstream of the translation stop codon. The available var flanking sequences from other strains than 3D7 varied between 250 and 2100 bp in length. For the most part 3′ sequences were those retrieved by Mercereau-Puijalon et al (Mercereau-Puijalon et al. 2002). Var gene domain structures were defined using definitions described in Smith et al. (Smith et al. 2000).
   DBLαCIDR1 domains were aligned from Pro-Cys (PC) of DBLα homology block A to the conserved Glu-Trp (EW) motif of CIDR M2 area, resulting in sequence lengths of 550 to 650 aa. For analysis of DBL relationships sequences covering Pro-X-Arg-Arg (PXRR) of DBL homology block B to Glu-Trp (EW) of homology block H were aligned.
m2-3. Distance trees were constructed the by p-distance/neighbour-joining (NJ) method as well as maximum parsimony (MP) using MEGA version 2.1 (Kumar et al. 2001). Trees were bootstrapped 1000 times and compared between NJ and MP tree-building methods to assure confidence in topology. Observed clusters from each tree were confirmed visually on alignments.

The 1.5 kb 5′ region of 3D7 var genes has previously been described to group into three major sequence groups, upsA, upsB and upsC (Gardner et al. 2002). To further investigate sequence similarities in this region, we analysed the 2 kb upstream sequences of all 3D7 var genes and the pseudo var gene PFE1640w. In agreement with Gardner et al. (Gardner et al. 2002) the alignments revealed three major sequence groups with high similarity between sequences of each group. However, two sequences did not align well with any of the groups and within the groups, subgroups could be identified. Thus, each of the groups were analysed separately.

Ten var genes had 5′ regions belonging to the upsA group and all but one were positioned head-to-head with a rif gene, the exception PF08_{—}0141 was head-to-head with another var gene (FIG. 10). Using a primer set targeting upsA 3D7 sequences around −900 bp from the translation initiation codon, we could PCR amplify products of the expected sizes in 3D7 genomic DNA, as well as in five of five field-isolates tested (data not shown). These data suggests that upsA regions not are unique to 3D7.

Alignments and tree-building (FIG. 11) of the 500 bp var 3′ regions divided most sequences into four clusters (A-D). 13 sequences fell outside these clusters, and the relationship between these sequences could not be confirmed by bootstrapping or comparison the two tree-building methods used.

Because most PfEMP1 molecules contain a semi-conserved head structure comprising of DBL1α and CIDR1, we restricted the analysis of coding sequences to these domains. In 3D7 all but one var gene encode a DBLIα as the first domain and in all but four genes DBL1 is followed by a CIDR1. Since alignment and tree constructions of DBL1 and CIDR1 domains individually yielded almost identical clusters, we decided to analyse the head structure sequences from the N-terminal region of DBL1 to a conserved motif in the C-terminal region of CIDR1 (FIG. 12). Fifty-two sequences, including that of pseudogene PFE1640w, could be grouped into five clusters, and four sequences could not be assigned any of these. When all CIDR sequences are aligned most CIDR1s fall into separate clusters of CIDRα or CIDRα1 domains (Robinson et al. 2003). The exceptions are three sequences (PF08-0141, PF11_{—}0008, PF13_{—}0003), which fall into a CIDRγ cluster. In FIG. 12, the head structures of these genes fall into group A. Robinson et al. (Robinson et al. 2003) found that most CIDR domains bind CD36 but identified nine, which did not. These constitute cluster A.

FIG. 9 schematically sums up the findings of all the var gene sequence analyses. The combination of clusters and chromosomal organization of the var genes indicate that var genes can be grouped into three major subgroups, var group A, B and C and two intermediate groups group B/A and group B/C, which appear to represent transitions between these three groups. The two genes previously shown to belong to conserved var families, var1 and var2, fell outside these groups. Group A var genes were most easily defined, whereas the borders of the proposed group B and C were less clear (FIG. 9A). The grouping was supported by analyses of both coding and non-coding sequences. However, the best predictors for the groups were the upstream region and chromosomal organization. Thus, genes placed near the telomere and with a transcriptional direction towards the telomere all had upsA sequences and formed group A. Group B were dominated by telomeric located but centromeric transcribed genes flanked by upsB and finally group C harboured all centromeric located genes with a upsC 5′ region.

Group A comprise most large PfEMP1s with a domain structure different from the most common 4-domain type, which is the predominant domain structure of Group B and C. Two genes PF08_{—}0140 and MAL6P1.316 were classified as B/A because they had upsBsh 5′ regions and chromosomal characteristics in common with group B genes, but had DBLα-CIDR1 sequences and domain structure characteristic for group A genes. Interestingly, these two genes are adjacent to a group A var or a pseudo var gene with an upsA region both transcribed in the opposite direction, thereby merging their 5′ regions. Adding the DBL1α-CIDR1 of the flanking pseudogene MAL6P1.317 to the alignments placed this pseudogene within DBLα-CIDR1 cluster A.

The fact that 5′ regions predict var gene chromosomal organisation and domain structure, and sequence similarities in coding and non-coding regions several thousand bases downstream from the translation initiation site implies that recombination, or other mechanisms of homogenizing exchange is much more likely to occur between var genes within a group than between var genes of different groupings. It can be proposed that an original ancestral var gene has been duplicated and diverged in the three main types, and each of these have then diverged into the genes of each group. In this process information may also have been exchanged between genes of different groupings. The data suggests that some exchange have taken place between groups B and C and some characteristics of group A have leaked into these groups, but that characteristics from groups B and C have not gained access to group A.

In conclusion, var genes can be sub-grouped into three major groups (group A, B and C) and two intermediate groups B/A and B/C representing transitions between the three major groups. The best defined var group, group A, comprises telomeric genes transcribed towards the telomere encoding PfEMP1s with complex domain structures different from the 4-domain type dominant of groups B and C. A rif subgroup transcribed towards the centromere was found neighbouring var genes of group A such that the rif and var 5′ regions merged. This organization appeared to be unique for the group A var genes.

### Example 3

### Selection of P. falciparum Isolate 3D7 for Expression of well Recognised VSA in vitro

Establishment of the genetic control of changes in VSA expression in response to in vitro selection is now possible because of the availability of the entire genomic sequence of the P. falciparum clone 3D7, which is a long-term clone derived from P. falciparum NF54 isolated from a Dutch malaria patient (Delemarre and Van der Kaay, 1979).

As a first step towards direct molecular identification of VSA_{SM}-encoding genes in 3D7, we established a method to enforce expression of VSA_{SM}-like antigens in this parasite clone by a novel selection method using plasma from semi-immune children with low levels of VSA_{UM}-IgG but high levels of VSA_{SM}-IgG (FIG. 5).

### Materials and Methods

m3-1. To enrich 3D7 for iRBC expressing VSA well recognized by IgG in the SM1 plasma pool, 1×10⁸ RBC infected with late trophozoite-stage parasites purified by gelatine flotation) were mixed with 200 μL pooled plasma in 3 mL culture medium, and incubated the mixture at room temperature (20-25° C.) for 15 min. Antibodies not reactive with intact iRBC were removed by washing×2 (800 g, 30 min). The iRBC were then resuspended in 3 mL culture medium and incubated as above with 100 μL biotinylated secondary antibody recognizing human IgG (Dako, Glostrup, Denmark). The iRBC were washed twice and resuspended as above and subsequently mixed with 100 μL streptavidin-coated DynaBeads (Dynal, Oslo, Norway). Finally, we isolated DynaBeads-coated iRBC by placing the IRBC suspension in a magnet field until all DynaBeads had settled at the magnet. The culture medium containing RBC and iRBC not covered with DynaBeads was removed by decantation. This procedure was repeated once before the DynaBeads-covered iRBC were transferred to a new culture bottle with medium and uninfected RBC for continuation of in vitro culturing.
m3-2. Human plasma samples used for selection: We used repeated rounds of panning on DynaBeads coated by IgG from two plasma pools (SM1, SM2) from semi-immune Ghanaian children and one plasma pool (SM3) from semi-immune Tanzanian children to select 3D7 parasites expressing VSA that were highly recognized by IgG in these plasma pools (Staalsoe et al., 2003).

Flow cytometry analysis of unselected 3D7 indicated that its VSA expression was heterogeneous but that the majority of the iRBC expressed VSA that were poorly recognised by IgG in the plasma of individuals living in areas of endemic P. falciparum transmission (FIG. 5). In particular, only few iRBC were specifically labelled by IgG in the plasma of semi-immune children with documented high levels of IgG with specificity for VSA expressed by P. falciparum isolated from patients with severe malaria (VSA_{SM}) and low levels of VSA expressed by patients with uncomplicated malaria (VSA_{UM}) (FIG. 5A). Although IgG levels in the plasma of sympatric adults were higher than in children, they were still moderate (FIG. 5B). Taken together, these data indicate that the VSA expressed by unselected 3D7 were of the VSA_{UM} type.

Only a minority of RBC infected by unselected 3D7 expressed VSA_{SM}-type antigens. To increase the proportion of iRBC expressing VSA_{SM}-type molecules in the culture, we first incubated 3D7 with the SM1 pool of plasma from semi-immune children, then with biotinylated anti-human IgG and finally with streptavidin-coated DynaBeads. As expected from the heterogeneous IgG recognition of VSA expressed by unselected 3D7, this resulted in some iRBC being covered by multiple DynaBeads while most were devoid of any beads (FIG. 6). We subsequently isolated the DynaBeads-covered iRBC by exposing the culture to a strong magnetic field, and used only these to propagate the culture further. Flow cytometry analysis showed that the IgG recognition of the VSA expressed by the 3D7_{SM1} sub-clone obtained by three rounds of selection in this manner was much stronger than that of VSA expressed by the unselected 3D7 parental clone (FIG. 5C-D). The biggest increase in IgG recognition of VSA following selection was seen in the plasma from children with documented high levels of VSA_{SM}-type IgG and low IgG levels with specificity for VSA_{UM}-type antigens (FIGS. 5A and 5C). This, and the fact that the level of IgG recognition of 3D7_{SM1} iRBC was both high and similar in children (FIG. 5C) and adults (FIG. 5D) show that the applied selection method had caused a change from predominantly VSA_{UM}-type antigens being expressed in unselected 3D7 to VSA_{SM}-type expression in 3D7_{SM1}.

The 3D7_{SM1} VSA_{SM}-expressing sub-clone was selected by its reactivity to IgG in a plasma pool from West African children living in an area of hyperendemic, seasonal P. falciparum transmission. To investigate whether the high VSA-specific IgG reactivity against 3D7_{SM1} extended beyond this region and in a variety of epidemiological settings, we measured levels of IgG with specificity for the VSA expressed by 3D7_{SM1} in series of plasma samples obtained from individuals (8 to 42 years of age) living in an area of hypoendemic, highly seasonal transmission in Sudan (FIG. 7A) and from children aged 3-11 years living in area of very intense transmission in Tanzania (FIG. 7B). All 20 Tanzanian children had medium-to-high levels of IgG unselected 3D7 iRBC and to all of five local parasite isolates, reflecting the high endemicity in their area of residence (FIG. 7B). Nevertheless, levels of IgG with specificity for the 3D7_{SM1} VSA were higher than those with specificity for unselected 3D7 (FIG. 7B). Importantly, levels of 3D7_{SM1} VSA-specific IgG were higher than IgG with specificity for VSA expressed by any of the local isolates, although 3D7_{SM1} was selected on IgG from West Africa. Taken together, these data suggest the existence of substantial conservation in time and space of the commonly and highly recognised VSA_{SM}-type iRBC surface antigens.

In conclusion, these findings open the possibility of identifying VSA of importance in the pathogenesis of severe disease (VSA_{SM}) by comparing VSA gene expression in isogenic parasites expressing VSA_{UM}- and VSA_{SM}-type iRBC surface molecules that are characterised by their differential IgG recognition pattern rather than by their adhesion specificity.

### Example 4

### 3D7 Parasite Expressing VSA_{SM} does not Adhere to CD36

Most members of the PfEMP1 family of 3D7 posses the capacity to bind CD36 and the binding motives have been mapped to the N-terminal part of the molecules. PfEMP1 that bind CD36 are structurally and functionally related (Robinson et al 2003) and belong to Groups B, C, or B/C (Lavstsen et al. 2003) and Example 2. In this example we show that 3D7 expressing a VSA_{UM} phenotype bind CD36, whereas 3D7 expressing the VSA_{SM} phenotype does not.

Materials and methods:
m4-1. We used Chinese hamster ovary (CHO) cells transfected to express human CD36 and cultured by standard methodology to measure iRBC adhesion to these receptors (Staalsoe et al. 1999; Hasler et al. 1993; Rogerson et al. 1995). In brief, parasites were radiolabelled by incubating the cultures overnight in the presence of ³H-phenylalanin (1 MBq for a standard culture containing 200 μl packed RBC). Wildtype and CD36 transfected CHO cells were grown to a monolayer in 96-well microtitre plates (Nunc, Roskilde, Denmark). Late-stage-enriched iRBC (100 μl, 1×10⁷ RBC/ml) were added to the CHO cell monolayer and incubated for one hour at 37° C. before unbound IRBC were washed away from the CHO cell monolayer. Finally, the number of CHO-adhering iRBC was determined by liquid scintillation spectrometry.

To investigate the relationship between VSA_{SM} expression and iRBC adhesion to CD36, we compared the ability of RBC infected by 3D7 and 3D7_{SM1} to adhere to CD36-transfected (CD36-CHO) CHO cells (FIG. 8). While 3D7 showed four- to 10-fold higher adhesion to CHO-CD36 cells than to wild-type CHO-0 cells, adhesion of 3D7_{SM1} to the transfected cells was never more than twice that to wild-type cells (FIG. 8). The average adhesion ratio was 30·3 for 3D7 and 1·4 for 3D7_{SM1}, corresponding to a difference of 4.9 [95% confidence interval: (1·9-7·9)]. These results indicate that there is a relationship between VSA_{SM} expression and the loss of ability of iRBC to adhere to CD36.

In conclusion, these results indicate that parasites expressing the VSA_{SM} phenotype do not adhere to CD36.

### Example 5

### Selection of P. falciparum Isolate 3D7 for Expression of Well Recognised VSA in vitro Results in Selective Up-Regulation of Group A var Genes

Parasite-encoded PfEMP1 proteins expressed on the surface membrane of iRBC mediate the adhesion of such erythrocytes to a range of host receptors. The PfEMP1 proteins are encoded by the var gene family containing 50-60 members per haploid parasite genome. Different PfEMP1 molecules have different receptor specificities, and clonal switching between expression of the various var gene products in a mutually exclusive manner allows the parasite to modify its adhesion properties. Gene expression and switching can be examined using gene-specific primers and real-time PCR.

Using real-time quantitative PCR we compared var gene expression in the parasite line 3D7 before (3D7_{UM}) and after (3D7_{SM}) selection for antibody recognition (Example 3),
m5-1. RNA was purified from 3D7_{UM} and 3D7_{SM} and used for the synthesis of cDNA. Total RNA was prepared with Trizol LS (http://www.invitrogen.com) as recommended by the manufacturer, and treated with DNAse1 (http://www.invitrogen.com). Absence of DNA in RNA samples was confirmed by stable base fluorescence after 40 cycles of real-time PCR with seryl-tRNA synthetase primers (Salanti et al., 2003)) (Table 1). One μg of DNA-free RNA was reversed transcribed using Superscript II and random hexamer primers ((http://www.invitrogen.com)) at 25° C. for 10 min, 42° C. for 50 min followed by 70° C. for 15 min in a total volume of 40 μl.

### Table 1

Primer sets used in real-time PCR assays to specifically amplify 59 var genes and one pseudogenes (underlined). Where several genes are listed next to a single primer set, primer targets in the listed genes were identical.

| | | | |
|---|---|---|---|
| Primer | | | |
| set | Forward primer | Reverse primer | Target gene(s) |

| | | | |
|---|---|---|---|
| 1 | TGCGCTGATAACTCACAACA | AGGGGTTCATCGTCATCTTC | PFA0005w |
| 4 | GACGAGGAGTCGGAAAAGAC | TGGACAGGCTTGTTTGAGAG | PF10_0001 |
| 5 | GTGCACCAAAAGAAGCTCAA | ACAAAACTCCTCTGCCCATT | PF10_0406 |
| 6 | GAGGCTTATGGGAAACCAGA | AGGCAGTCTTTGGCATCTTT | PF11_0007 |
| 7 | GACGGCTACCACAGAGACAA | CGTCATCATCGTCTTCGTTT | PF11_0008 |
| | | | (SEQ ID NO.: 3) |
| 8 | TGCTGAAGACCAAATTGAGC | TTGTTGTGGTGGTTGTTGTG | PF11_0521 |
| 9 | TCGATTATGTGCCGCAGTAT | TTCCCGTACAATCGTATCCA | PFL0020w |
| 10 | TGGTGATGGTACTGCTGGAT | TTTATTTTCGGCAGCATTTG | PFL0030c |
| 11 | GACGCCTGCACTCTCAAATA | TTGGAGAGCACCACCATTTA | PFL0935c |
| 12 | AGCAAAATCCGAAGCAGAAT | CCCACAGATCTTTTCCTCGT | PFL1950w |
| 15 | CATCCATTACGCAGGATACG | AAATAGGGTGGGCGTAACAC | PFL1960w |
| 17 | GGCACGAAGTTTTGCAGATA | TTTGTGCGTCTTTCTTCGTC | PFL2665c |
| 18 | CGGAGGAGGAAAAACAAGAG | TGCCGTATTTGAGACCACAT | PFL0005w |
| 19 | CGGAATTAGTTGCCTTCACA | CATTGGCCACCAAGTGTATC | PF13_0364 |
| 20 | CACAGGTATGGGAAGCAATG | CCATACAGCCGTGACTGTTC | PF13_0003 |
| | | | (SEQ ID NO.: 5) |
| 21 | CAATTTTGGGTGTGGAATCA | CACTGGCCACCAAGTGTATC | PFB1055c |
| 22 | ATGTGCGCTACAAGAAGCTG | TTGATCTCCCCATTCAGTCA | PFB0010w |
| 23 | CAATCTGCGGCAATAGAGAC | CCACTGTTGAGGGGTTTTCT | PFC1120c/PFC0005w |
| 25 | ATATGGGAAGGGATGCTCTG | TGAACCATCGAAGGAATTGA | PFD0020c |
| 26 | ACCGCCCCATCTAGTGATAG | CACTTGGTGATGTGGTGTCA | PFD0615c |
| 27 | TAAAAGACGCCAACAGATGC | TCATCGTCTTCGTCTTCGTC | PFD0625c |
| 28 | ACTTTCTGGTGGGGAATCAG | TTCACCGCCACCTACTTCAG | PFD0630c/PFD0635c |
| 30 | GACGACGATGAAGACGAAGA | AGATCTCCGCATTTCCAATC | PFD0005w |
| 32 | ACCTAATGGCGAAAACCAAG | ACACTTGCCTTCATCCACTG | PFD1000c |
| 34 | TGCAACGAAACATTAGCACA | AGCAGGGGATGATGCTTTAC | PFD1015c |
| 35 | AAACACGTTGAATGGCGATA | GACGCCGAGGAGGTAAATAG | PFD1235w/MAL7P1.1 |
| | | | (SEQ ID NO.: 1) |
| 36 | TGACGACTCCTCAGACGAAG | CTCCACTGACGGATCTGTTG | PFD1245c |
| 37 | AAGAAAGTGCCACAACATGC | GTTCGTACGCCTGTCGTTTA | PFE1640w |
| 38 | GAAGCTGGTGGTACTGACGA | TATTTTCCCACCAGGAGGAG | PFE0005w |
| 39 | ATTTGTCGCACATGAAGGAA | AACTTCGTGCCAATGCTGTA | MAL6P1.252 |
| 41 | GGTGTCAAGGCAGCTAATGA | TATGTCCTGCGCTATTTTGC | PF08_0141 |
| 43 | GTCGTGGAAAAACGAAAGGT | TATCTATCCAGGGCCCAAAG | PF08_0142 |
| 44 | ATGTGTGCGAGAAGGTGAAG | TGCCTTCTAGGTGGCATACA | MAL6P1.4 |
| 45 | CAATTTTTCCGACGCTTGTA | CACATATAGCGCCGTCCTTA | PF07_0048 |
| 46 | GCGACGCTCAAAAACATTTA | TCATCCAACGCAATCTTTGT | PF07_0050 |
| 49 | GTTGAGTCTGCGGCAATAGA | CTGGGGTTTGTTCAACACTG | PF07_0049 |
| 50 | CACACATGTCCACCACAAGA | ACCCTTCTGTGGTGTCTTCC | MAL7P1.56 |
| 51 | ACGTGGTGGAGACGTAAACA | CCTTTGTTGTTGCCACTTTG | MAL7P1.55 |
| 52 | CGTGGTAGTGAAGCACCATC | CCCACCTTCTTGTGGTTTCT | PF07_0051 |
| 53 | TGACGACGATAAATGGGAAA | TTCTTTTGGAGCAGGGAGTT | PF07_0139 |
| 54 | ACCAAGTGGTGACAAAGCAG | GGGTGGCACACAAACACTAC | PFD1005c/PFD1015c |
| 55 | TTTGTCCGGAAGACGATACA | ATCTGGGGCAGAATTACCAC | PF08_0106 |
| 58 | CACACGTGGACCTCAAGAAC | AAAACCGATGCCAATACTCC | PFI1830c |
| 66 | CCTAAAAAGGACGCAGAAGG | CCAGCAACACTACCACCAGT | PF08_0107 |
| 67 | AAGGGAAGATTGGTGGACAG | AGGGGGATCAGTATCACGTC | MAL6P1.314 |
| 91 | ACAAAGGAACGTCCATCTCC | GCCAATACTCCACATGATCG | PF13_0001 |
| 92 | TGCAAGGGTGCTAATGGTAA | CCTGCATTTTGACATTCGTC | PF08_0103 |
| 93 | GACAAATACGGCGACTACGA | TGTTTCACCCCATTCTTCAA | MAL6P1.1 |
| 94 | TGGAAAGAACATGGACCTGA | TTCCTCGAGGGAAGAATCAC | MAL6P1.316 |
| 95 | TCACAACCTGACCCCCTACT | TCTTCGTCGTTGTCATCCTC | PFD0995c |
| 96 | TGACCAAGACGAAGTATGGAA | TTGATCTCTGTTCGCTGTCC | PFI1820w |
| 97 | TCATTATGGGAAGCACGATT | TGATTTCTACCATCGCAAGG | PFA0015c |
| 98 | ATGGTGGCAAACTTGTGAGA | TCCAATTGGTCTCCTTGACA | PF08_0140 |
| 99 | AGGGAGCATCAGGTGGTAGT | GCTGTGCATGCTTTTTCATT | MAL7P1.50 |
| 100 | GATCAAAAGAGGCGGAGAAG | TTCCAATTGGGGAATTTTTC | PFI0005w |
| 101 | CAAGAGACACAACCGGAAGA | CACTTCCAATTGGGGAATTT | PFA0765c |
| 60* | AAGTAGCAGGTCATCGTGGTT | TTCGGCACATTCTTCCATAA | PF07_0073 |

*Endogenous control gene: seryl-tRNA synthetase (PF07_0073)

To study gene expression of individual var genes a specific primer set for each of 59 var genes and one pseudogenes in the 3D7 genome was designed (Table 1), and real-time PCR was performed on cDNA from 3D7_{UM} and 3D7_{SM}.
m5-2.Real-time PCR was done using a Rotorgene thermal cycler system (http://www.corbettresearch.com). Change in PFD1235w/MAL7P1.1 (SEQ ID NO.: 1), PF11_{—}0008 (SEQ ID NO.: 3), and PF13_{—}0003 (SEQ ID NO.: 5) gene transcription after selection for antibody recognition. Specific primers targeting SEQ ID NO.: 1, SEQ ID NO.: 3, and SEQ ID NO.: 5 (FIGS. 13 and 14) were used to measure transcription before (3D7_{UM}) and after antibody selection (3D7_{SM}). The fold change in transcription levels, normalised against seryl-tRNA synthetase, was calculated for each primer set by the ΔΔCt method (User Bulletin #2, Applied Biosystems, http://www.appliedbiosystems.com). Reactions were performed in 20 μl volumes using QuantiTect SYBR Green PCR master mix and 0.5 mM primers, according to manufacturer's instructions (http://www.giaaen.com). PCR cycling conditions optimised for P. falciparum cDNA were 95° C. for 15 min followed by 40 cycles of 94° C. for 30 sec, 54° C. for 40 sec, and 68° C. for 50 sec with a final extension at 68° C. for 10 min. Data acquisition was done at the end of elongation of each cycle. Specificity of amplification was ascertained by melting-curve analysis of each PCR product.
   Electrophoresis of PCR products and EB staining was performed and revealed no bands from no-template controls and single bands for all targets in cDNA PCR products. Quantification was done using the Rotorgene software version
   4.30 (http://www.giagen.com). Transcription levels of the endogenous P. falciparum genes actin, seryl-tRNA synthetase and aldolase were analysed in order to determine the most accurate endogenous control. P. falciparum seryl-tRNA synthetase displayed the most uniform transcription profile in different parasite isolates and an unchanged pattern throughout the parasite life and was thus used for calculations of fold changes in var gene transcription by the ACT method (described in User Bulletin #2, Applied Biosystems, http://www.appliedbiosystems.com).
m5.3. Genes (PF11_{—}0521, PFL0030c, PFL1950w, PFL005w, PF13_{—}003 (SEQ ID NO.: 5), PFB1055c, PFD1235w/MAL7P1.1 (SEQ ID NO.: 1), PFE1640w, PF11830, PF08_{—}0107, MAL6P1.314, MAL6P1.316, PFD0995c, PFA0015c, seryl-tRNA synthetase) used for absolute quantification were PCR amplified, ligated into the pCR2.1 TOPO vector and transformed into Eschericia coli TOPO10 cells (TA cloning System, Invitrogen). Plasmids were purified using Qiagen Miniprep spin columns (Qiagen, Merck Eurolab, Albertslund, Denmark) and the identity of inserts verified by subsequent sequencing on an ABI Prism 310 (Perkin-Elmer) using the Big Dye terminator reaction mix, ABI Prism proofreading and translation software, and the P. falciparum 3D7 genome database (http://www.plasmodb.org).
   Plasmid concentrations were determined by spectrophotometry and serial 10-fold Tris-EDTA buffer (pH=7.5) dilutions ranging from 5×10⁸ to 5×10⁰ template copies per real-time PCR reaction. Triplicate real-time measurements were made for each dilution and a best-fit standard curve was generated using the RotorGene 2000 software. The standard curves were linear across a range of seven logs of DNA concentrations with correlation coefficients between 0.9779 and 0.9969. The detection limit of the system was ≧20 copies (data not shown). The coefficient of variance (CV) was calculated as 100*(standard deviation/mean). RT-PCR was performed as described using 1 μg total RNA in a total volume of 40 μl of which 0.5 μl was subsequently used for real-time PCR. Absolute values were calculated from the standard curves.

Real-time PCR followed by calculating fold change in 3D7_{UM} compared to 3D7_{sM} demonstrated marked upregulation of three Group A var genes PFD1235/MAL7P1.1w (SEQ ID NO.: 1), PF11_{—}0008 (SEQ ID NO.: 3), and PF13_{—}0003 (SEQ ID NO.: 5). The transcription of Group B, C or B/C var genes was either downregulated or not regulated in the 3D7_{SM} compared to 3D7_{UM} (FIGS. 13 and 14, Table 2). The up-regulated var genes PFD1235w/MAL7P1.1 (SEQ ID NO.: 1), PF11_{—}0008 (SEQ ID NO.: 3), and PF13_{—}0003 (SEQ ID NO.: 5) were also the most dominant var transcript when doing absolute quantification (Table 2) with var gene-specific primers. The upregulated and dominant PFD1235w/MAL7P1.1, PF11_{—}0008 (SEQ ID NO.: 3), and PF13_{—}0003 (SEQ ID NO.: 5) gene showed 3 to 10-fold higher level of expression following antibody selection both in ring-stage and trophozoite/schizont-stage 3D7 parasites (Table 2).

TABLE 2
Copy number of selected var genes and seryl-tRNA synthetase transcripts in unselected and VSA_{UM}-expressing 3D7 and 3D7
after antibody-selection for VSA_{SM} expression

| | | | | | | | | Ring-stage parasites | | | | |
|---|---|---|---|---|---|---|---|---|---|---|---|---|
| | | | Trophozoite/schizont-stage parasites | | | | | | | Antibody- | | |
| | | | Unselected | | Antibody-selected | | Fold | Unselected | | selected | | Fold |
| Cluster^{a} | Primer^{b} | Gene | Copies | CV^{c} | Copies | CV^{c} | change | Copies | CV^{c} | Copies | CV^{c} | change |
| A | 8 | PF11_0521 | 426 | 11 | 7,119 | 27 | 16.7 | 12,570 | 31 | 84,341 | 29 | 6.7 |
| | 20 | PF13_0003 | 1,797 | 7 | 6,129 | 20 | 3.4 | 77,934 | 13 | 179,746 | 9 | 2.3 |
| | 35 | PFD1235w/MAL7P1.1 | 2,931 | 20 | 29,941 | 13 | 10.2 | 101,417 | 27 | 1,152,758 | 12 | 11.4 |
| | 67 | MAL6P1.314 | 3,145 | 13 | 19,356 | 14 | 6.2 | 87,130 | 19 | 767,747 | 8 | 8.8 |
| | 97 | PFA0015c | 77,890 | 12 | 1,333 | 30 | 58.4 | 155,151 | 21 | 18,419 | 14 | 8.4 |
| B/A | 94 | MAL6P1.316 | 13,742 | 15 | 36,637 | 24 | 2.7 | 247,156 | 29 | 1,057,056 | 28 | 4.3 |
| B | 18 | PFL0005w | 15 | 21 | 1,106 | 23 | 73.7 | 928 | 12 | 13,769 | 23 | 14.8 |
| | 21 | PFB1055c | 64 | 16 | 2,533 | 12 | 39.6 | 5,272 | 22 | 26,070 | 5 | 4.9 |
| | 58 | PFI1830c | 79,111 | 39 | 59,860 | 22 | 1.3 | 18,600 | 15 | 13,124 | 12 | 1.4 |
| B/C | 12 | PFL1950w | 226 | 22 | 524 | 8 | 2.3 | 13,236 | 19 | 19,053 | 24 | 1.4 |
| C | 66 | PF08_0107 | 865,266 | 33 | 58,833 | 28 | 14.7 | 10,459,156 | 21 | 611,814 | 15 | 17.1 |
| | 95 | PFD0995c | 23,415 | 19 | 491 | 12 | 47.7 | 567,205 | 19 | 9,148 | 19 | 62 |
| var2 | 10 | PFL0030c | 907 | 14 | 12,562 | 19 | 14 | 12,905 | 30 | 127,631 | 30 | 9.9 |
| var1 | 37 | PFE1640w | 12,279 | 21 | 14,239 | 10 | 1.2 | 8,224 | 17 | 9,111 | 30 | 1.1 |
| | | seryl-tRNA | 609,533 | 13 | 609,533 | 13 | 1 | 643,271 | 20 | 643,271 | 20 | 1 |
| | | synthetase | | 0 | | 0 | | | | | | |

^{a}As described in Lavstsen et al., 2003;
^{b}Salanti et al. 2003 and Table 1;
Coefficient of variation (%) between three different quantification experiments

In conclusion, IgG selection resulted in marked upregulation of SEQ ID NO.: 1, 3, and 5.

### Example 6

Selection of P. falciparum Isolate 3D7 for Adhesion to Human Endothelial Cells in vitro Results in Selective Up-Regulation of Group A var Genes

The particular virulence of P. falciparum is due to the ability of infected erythrocytes to adhere to a variety of host receptors on the endothelial lining such as ICAM-1, VCAM, thrombospondin, ELAM-1, and CD36, and avoid splenic clearance. To show that selection of 3D7 on human endothelial cells leads to a serological phenotype and a transcription profile similar to 3D7_{SM1} we did several rounds of selection on bone marrow derived endothelial cells generating 3D7_{endo}.
m6-1. We used human endothelial cells to select 3D7 for increased VSA_{SM} expression.

Human endothelial cells were cultured by standard methodology to measure iRBC adhesion to receptors on the endothelial cells. In brief, parasites were radiolabelled by incubating the cultures overnight in the presence of ³H-phenylalanin (1 MBq for a standard culture containing 200 μl packed RBC). Late-stage-enriched iRBC (100 μl, 1×10⁷ RBC/ml) were added to the endothelial cell monolayer and incubated for one hour at 37° C. before unbound iRBC were washed away from the endothelial cell monolayer. Finally, the number of endothelial cell-adhering iRBC was determined by liquid scintillation spectrometry.
m6-2. Real-time PCR was done using a Rotorgene thermal cycler system (http://www.corbettresearch.com). Change in PFD1235w/MAL7P1.1 (SEQ ID NO.: 1), PF11_{—}0008 (SEQ ID NO.: 3), and PF13_{—}0003 (SEQ ID NO.: 5) gene transcription after selection for antibody recognition. Specific primers targeting SEQ ID NO.: 1, 3, and 5 (FIG. 20) were used to measure transcription before (3D7_{UM}) and after antibody selection (3D7_{sM}). The fold change in transcription levels, normalised against seryl-tRNA synthetase, was calculated for each primer set by the ΔΔCt method (User Bulletin #2, Applied Biosystems, http://www.appliedbiosystems.com). Reactions were performed in 20 μl volumes using QuantiTect SYBR Green PCR master mix and 0.5 mM primers, according to manufacturer's instructions (http://www.gliagen.com). PCR cycling conditions optimised for P. falciparum cDNA were 95° C. for 15 min followed by 40 cycles of 94° C. for 30 sec, 54° C. for 40 sec, and 68° C. for 50 sec with a final extension at 68° C. for 10 min. Data acquisition was done at the end of elongation of each cycle.

Specificity of amplification was ascertained by melting-curve analysis of each PCR product. Electrophoresis of PCR products and EB staining was performed and revealed no bands from no-template controls and single bands for all targets in cDNA PCR products. Quantification was done using the Rotorgene software version 4.30 (http://www.giagen.com). P. falciparum seryl-tRNA synthetase was used for calculations of fold changes in var gene transcription by the ACT method (described in User Bulletin #2, Applied Biosystems, http://www.appliedbiosystems.com).

The resulting cell line 3D7_{endo} showed a FACS profile similar to that of 3D7_{SM1} (Example 3 and FIG. 5) and a marked up-regulation of the PFD1235w/MAL7P1.1 (SEQ ID NO.: 1), PF11_{—}0008 (SEQ ID NO.: 3), and PF13_{—}0003 (SEQ ID NO.: 5) var genes as analysed by real-time quantitative PCR (FIG. 20).

In conclusion, we find that selection of 3D7 for adhesion to human endothelial cells confer a serological phenotype, VSA_{SM} similar to that of parasites causing severe malaria and to 3D7 selected with childrens plasma. As in 3D7_{SM1} the acquisition of the VSA_{SM} phenotype in 3D7_{endo} is accompanied by marked upregulation of the PFD1235w/MAL7P1.1 (SEQ ID NO.: 1), PF11_{—}0008 (SEQ ID NO.: 3), and PF13_{—}0003 (SEQ ID NO.: 5) var genes.

### Example 7

### Northern Blots of Antibody-Selected, Ring-Stage 3D7 Showed Strong Hybridization Signals Corresponding to Full-Length Transcripts of PFD1235W/MAL7P1.1 (SEQ ID NO.: 1)

### Materials and Methods:

m7-1. For Northern blotting, we used 10 μg of total RNA separated in a standard denaturing MOPS-formaldehyde agarose gel and transferred to positively charged nylon membranes overnight (Sambrook et al., 1989). RNA was cross-linked to the membrane by baking for 30 min at 120° C. DIG-labeled RNA probes were generated using the DIG RNA labeling kit (Roche, Hvidovre, Denmark). Hybridization, washing, and detection were done according to the manufacturer's recommendations with a hybridization temperature of 65° C. in DIG Easy Hyb buffer (Roche).

Northern blots of antibody-selected, ring-stage 3D7 showed strong hybridization signals corresponding to full-length transcripts of the upregulated var gene PFD1235w/MAL7P1.1 (SEQ ID NO.: 1) (FIG. 15A). The corresponding signals were either absent or much weaker in Northern blots of unselected 3D7. Similarly, PF11_{—}0008 (SEQ ID NO.: 3) and PF13_{—}0003 (SEQ ID NO.: 5) were found to be present as full-length transcripts in antibody-selected 3D7, but not in 3D7 (data not shown).

Taken together, these results shows that antibody-selected 3D7 expressing VSA_{SM}-type antigens transcribe full-length PFD1235w/MAL7P1.1 (SEQ ID NO.: 1), PF11_{—}0008 (SEQ ID NO.: 3) and PF13_{—}0003 (SEQ ID NO.: 5).

### Example 8

The PFD1235w/MAL7P1.1 (SEQ ID NO.: 1), PF11_{—}0008 (SEQ ID NO.: 3), and PF13_{—}0003 (SEQ ID NO.: 5) genes that is selectively upregulated in P. falciparum isolate 3D7 following selection for antibody recognition in vitro belong to the Group A var genes and share their characteristics

From Example 3, 4, 5, and 6 it appears that the Group A PFD1235w/MAL7P1.1 (SEQ ID NO.: 1), PF11_{—}0008 (SEQ ID NO.: 3) and PF13_{—}0003 (SEQ ID NO.: 5) genes encodes VSA_{SM}-like proteins, and that this protein is involved in the acquisition of the VSA_{SM} phenotype following antibody-selection of VSA_{UM}-expressing 3D7 (3D7_{SM}). The fact that almost identical results were obtained using plasma pools from children living in West Africa (pools SM1 and SM2) and East Africa (plasma pool SM3) indicate that a PfEMP1 similar to that encoded by PFD1235w/MAL7P1.1 (SEQ ID NO.: 1), PF11_{—}0008 (SEQ ID NO.: 3) and PF13_{—}0003 (SEQ ID NO.: 5) are present in P. falciparum parasites transmitted on both sides of this vast continent.

In conclusion, PFD1235w/MAL7P1.1 (SEQ ID NO.: 1), PF11_{—}0008 (SEQ ID NO.: 3) and PF13_{—}0003 (SEQ ID NO.: 5) seems to belong to a sub-group of var genes, with a constrained ability to recombine, that are functionally conserved and commonly recognised by VSA_{SM}-antibodies.

### Example 9

### PFD1235w/MAL7P1.1 (SEQ ID NO.: 1), PF11_{—}0008 (SEQ ID NO.: 3) and PF13_{—}0003 (SEQ ID NO.: 5) Belongs to a var Sub-Family that is Common and Highly Conserved in P. falciparum Isolates

PFD1235w/MAL7P1.1 (SEQ ID NO.: 1), PF11_{—}0008 (SEQ ID NO.: 3) and PF13_{—}0003 (SEQ ID NO.: 5) are the dominant transcript and is highly upregulated in the P. falciparum isolate 3D7 following selection for antibody selection and adhesion to human endothelial cells (3D7_{SM} and 3D7_{endo}; Example 3, 4, 5, and 6). All the 3D7 var genes differ from each other, but smaller blocks of sequences with high similarity are found in various var genes. To date, only three sub-families of PfEMP1 have been defined (var1-3) (Salanti et al. 2002; Salanti et al. 2003; Kraemer and Smith, 2003). Apart from the var1-3 sub-families, all PfEMP1 genes described so far from other parasite isolates differ from each other, and from the 3D7 var genes. It has therefore been assumed that the global repertoire of var genes is very large. This constitutes an obvious obstacle for the development of vaccines based on var genes and their products, as a high degree of conservation is a prerequisite for vaccine pan-reactivity.

To test the degree of inter-genomic diversity of PFD1235w/MAL7P1.1 (SEQ ID NO.: 1), PF11_{—}0008 (SEQ ID NO.: 3) and PF13_{—}0003 (SEQ ID NO.: 5); 60 different Ghanaian parasite isolates obtained from the peripheral blood of P. falciparum malaria patients were tested.
m9-1. Genomic DNA was isolated (http://www.clontech.com) using the NucleoSpin purification kits according to the manufacturer's recommendations. PCR was carried out in 0.2-ml microfuge tubes in a reaction volume of 20 μl using a PE2400 PCR machine (http://www.perkin-elmer.com). Final concentrations of the PCR reagents

were as follows: Hotstart Taq polymerase (http://www.giagen.com): 0.1 U; primers: 1 μM; dNTP: 2.5 mM, each; and MgCl₂: 1.5 mM). Cycling conditions were optimised for P. falciparum DNA: 15 min at 95° C. followed by 30 cycles of 30 sec at 94° C., 30 sec at 50° C., and 4 min at 68° C., with a final extension for 15 min at 68° C. The PCR products were visualised and size was determined in a 1% agarose gel containing EB. PCR amplification using PFD1235w/MAL7P1.1, PF11_{—}0008, and PF13_{—}0003 specific primers (Table 1) on genomic DNA from 60 isolates from Ghanaian children yielded a definite band of the expected size of 160 bp in 10, 3, 20, respectively of the isolates.
m9-2. To demonstrate the extent of sequence similarity, 2304 bp corresponding to 768 amino acids were cloned and sequenced. Gene-specific primers for PFD1235w/MAL7P1.1 (SEQ ID NO.: 1) were used to perform PCR on genomic DNA from PCR positive isolates. PCR products were gel-purified using the Qiagen gel purification kit according to the manufacturer's instructions (http://www.giaqen.com). Purified PCR fragments were ligated into the PCRII TOPO vector using TOPO TA cloning kit, and transformed into TOP10 competent cells (http://www.invitrogen.com). Positive clones were selected and propagated. Plasmid preparations were made using MiniPrep spin columns (http://www.giagen.com). Sequencing was performed on an ABI Prism 377 using the Big Dye terminator reaction mix (http://www.perkin-elmer.com). Proofreading and translation were done with ABI Prism software.

From the sequencing it was found that 556/768 cloned from BM021 and BM048 were identical to the PFD1235W/MAL7P1.1 (SEQ ID NO.: 1) sequence (FIG. 19). Alignments were done using ClustalW and default settings.

Taken together, these data show that PFD1235w/MAL7P1.1 (SEQ ID NO.: 1) belongs to a conserved and common gene sub-family and thus fulfils two required criteria for any candidate gene in vaccine development. Similarly PF11_{—}0008 (SEQ ID NO.: 3) and PF13_{—}0003 (SEQ ID NO.: 5) were found to belong to var gene sub-families families with similar identities.

### Example 10

### 3D7_{SM1} Expresses VAR4 (SEQ ID NO.: 2) on the Surface of Infected Erythrocytes

To generate recombinant proteins of VAR4 (SEQ ID NO.: 2) domains were cloned into the pBAD-TOPO vector (Invitrogen) by PCR using the following domain-specific oligonucleotide primers and subsequently into the Baculovirus transfer vector pAcGP67:

| | |
|---|---|
| NTS.Fw: | 5′-GAATTCATGGGGAATGCATCATCA-3′ |
| NTS.Rv: | 5′-ATTTCTATCTTCTGCGTT-3′ |
| DBL1-α.Fw: | 5′-GAATTCAACGCAGAAGATAGAAATC-3′ |
| DBL1-α..Rv: | S′-CTTCGATGTATATGTCTG-3′ |
| CIDR1-α.Fw: | 5′-GAATTCGACGCTAAAACTGATAGTA-3′ |
| CIDR1-α.Rv: | 5′-ACATATATCGCCATTCAACG-3′ |
| DBL2-β.Fw: | 5′-GAATTCTTGAATGGCGATATATGTA-3′ |
| DBL2-β.Rv: | 5′-GTATATAGCTGATACTGT-3′ |
| C2.Fw: | 5′-GAATTCACAGTATCAGCTATATACC-3′ |
| C2.Rv: | 5′-ATAACATGGTTGTTGTGA-3′ |
| DBL3-β..Fw: | 5′-GAATTCTCACAACAACCATGTTATG-3′ |
| DBL3-β..Rv: | 5′-ATCTATTCCACCTGTAGT-3′ |
| DBL4-γ.Fw: | 5′-GAATTCACTACAGGTGGAATAGATCA-3′ |
| DBL4-γ.Rv: | 5′-ACATGCGGCATTGAGACT-3′ |
| DBL5-δ.Fw: | 5′-GAATTCAGTCTCAATGCCGCATGTG-3′ |
| DBL5-δ.Rv: | 5′-TCTACAATGTCTGGCACA-3′ |
| CIDR2-β.Fw: | 5′-GAATTCTGTGCCAGACATTGTAGATC-3′ |
| CIDR2-β.Rv: | 5′-TTTGCCACTAGGTACGT-3′ |

m10-1. For production of carboxy-terminally V5 epitope and histidine-tagged protein the PCR
   amplified inserts were excised from pBAD-TOPO constructs by EcoRI and PmeI digestion and subsequently sub-cloned into the EcoRI and blunt-ended BglII sites of the Baculovirus transfer vector pAcGP67-A (BD Biosciences, Brøndby, Denmark). Recombinant Baculovirus were generated by co-transfection of the pAcGP307-A-NTS,
   pAcGP67-A-DBL1-α, pAcGP67-A-C1DR15-α, pAcGP67-A-DBL2-β, pAcGP67-A-C2, pAcGP67-A-DBL3-β, pAcGP67-A-DBL4-γ, pAcGP67-A-DBL5-δ, or pAcGP67-A-CIDR2-β D construct genes and Bsu330I linearized Bakpak6 Baculovirus DNA (BD Biosciences Clontech) into insect Sf9 cells. Recombinant products were expressed by infection of insect High-Five cells with recombinant Baculovirus using a multiplicity of infection of ten. Recombinant proteins were purified from culture supernatants on Co2+ metal-chelate agarose column and eluted with 25 mM HEPES-KOH (pH=7.30), 0.5 mM MgCl₂, 0.5 mM DTT, 100 mM NaCl, 10% glycerol, and 100 mM imidazole.
m10-2. For production of GST fusion protein a conserved sequence of the intracellular acidic
   segment ATS/Exon2 was sub-cloned into the pGEX-4T1 vector by PCR using the following oligonucleotide primers ATS-Fw:
   5′-CGGAATTCAAAACAAAATCATCAGTAG-3′,
   ATS-Rv: 5′-ATAAGAATGCGGCCGCGTTGATTACCACTTAATGTG-3′. The proteins were expressed as fusion proteins at the carboxy-terminus of glutathione S-transferase from Schistosoma japonicum and purified by affinity chromatography on glutathione sepharose 4B (Amersham Pharmacia Biotech).
m10-3. To generate murine antibodies against VAR4 domains and ATS/Exon2 the recombinant
   proteins were used to immunize Balb/c mice (5 μg) and rabbit s (50 μg) (given subcutaneously in Freund's complete adjuvant) followed by two booster injections in Freund's incomplete adjuvant.

A DNA vaccination approach to generate antibodies to VAR4 (SEQ ID NO.: 2) domains were also used. All domains was cloned into the Eucaryotic TA expression vector pCR3.1 (Invitrogen) using the following primers:

| | |
|---|---|
| DBL1-α.Fw: | 5′-GCCRCCATGGACGCAGAAGATAGAAATC-3′ |
| DBL1-α..Rv: | 5′-CTACTTCGATGTATATGTCT-3′ |
| CIDR1-α.Fw: | 5′-GCCRCCATGGACGCTAAAACTGATAGTA-3′ |
| CIDR1-α.Rv: | 5′-CTAACATATATCGCCATTCAACG-3′ |
| DBL2-β.Fw: | 5′-GCCRCCATGGTGAATGGCGATATATGTA-3′ |
| DBL2-β.Rv: | 5′-CTAGTATATAGCTGATACTGT-3′ |
| C2.Fw: | 5′-GCCRCCATGGCAGTATCAGCTATATACC-3′ |
| C2.Rv: | 5′-CTAATAACATGGTTGTTGTGA-3′ |
| DBL3-β..Fw: | 5′-GCCRCCATGGCACAACAACCATGTTATG-3′ |
| DBL3-β..Rv: | 5′-CTAATCTATTCCACCTGTAGT-3′ |
| DBL4-γ.Fw: | 5′-GCCRCCATGGCTACAGGTGGAATAGATCA-3′ |
| DBL4-γ.Rv: | 5′-CTAACATGCGGCATTGAGACT-3′ |
| DBL5-δ.Fw: | 5′-GCCRCCATGGGTCTCAATGCCGCATGTG-3′ |
| DBL5-δ.Rv: | 5′-CTATCTACAATGTCTGGCACA-3′ |
| CIDR2-β.Fw: | 5′-GCCRCCATGGGTGCCAGACATTGTAGATC-3′ |
| CIDR2-β.Rv: | 5′-CTATTTGCCACTAGGTACGT-3′ |

m10-4. Plasmids were propagated in TOP10 cells (Invitrogen) and plasmid was purified using Plasmid GIGA prep kit (Qiagen). Plasmid DNA was injected IM to mice 4 times with 2 weeks intervals and finally boosted with the recombinant protein corresponding to the domain.
m10-5. SDS-extracted trophozoite/schizont-stage iRBC were reduced by boiling in the presence of L-mercaptoethanol and electrophoresed in the Laemmli sodium dodecyl sulfate polyacrylamide gel electrophoresis (SDS-PAGE) system (Sambrook et al., 1989). Immunoblots were prepared on PVDF membranes by semidry blotting using standard methods. All available binding sites on membranes were blocked in Tris-buffered-saline-Tween (TBS-T) containing 5% skimmed milk. Blots were probed with an antiserum raised against a recombinant protein of the highly conserved intracellular acidic segment ATS/Exon2 conserved among most var genes in 3D7, DBL5-δ of PFD1235w/MAL7P1.1 (SEQ ID NO.: 1), and pre-immune mouse sera diluted 1:100 in TBS-T. Bound antibody was detected with relevant IgG alkaline phosphatase-conjugated antibody (Dako, Glostrup, Denmark). All washes were done using TBS-T. Blots were developed by standard methods using p-nitroblue tetrazolium phosphate (NBT, Sigma) and 5-bromo-4-chloro-3-indolylphosphate (BCIP, Sigma) as substrates.
m10-6. Immunostaining and flow cytometry were performed as described (Staalsoe et al., 1999; Nielsen et al., 2002) with some modifications. Briefly, 2.5×10⁵ of MACS purified, ethidium bromide labeled iRBC were incubated for 1 hr in 20 μL of murine sera or for 30 min in 5 μL of human sera. All murine sera had been depleted of anti-human erythrocyte antibodies by absorption. For immunostaining with murine sera, iRBC were sequentially exposed to 100 μL of 1:25 diluted goat anti-mouse Ig (Dako), biotinylated anti-goat Ig (Dako) and 1:200 diluted FITC-conjugated streptavidin (Dako) for 30 minutes each. For immunostaining with human sera, iRBC were incubated in 100 μL of 1:25 diluted biotinylated anti-human IgG (Dako) then in 1:2000 diluted FITC-conjugated streptavidin for 30 min each time. For fluorescence and confocal microscopy, wet mounts of immunostained parasites were prepared and images were obtained using a Leica DM LB2 and a Carl Zeiss Scanning Microscope, respectively. For both microscopes, suitable filters and channels were used to detect FITC and ethidium-bromide staining.
   When appropriate, trypsin treatment was performed as described (Fernandez et al., 1999) with modifications. Cells were washed once in PBS, incubated in 10 volumes of 100 μg/ml TPCK-treated trypsin (Amersham Pharmacia Biotech) in PBS for 10 min at 37° C. The reaction was stopped with 1 volume of 2 mg/ml soybean trypsin inhibitor (Sigma-Aldrich) in RPMI 1640/5% Albumax. Cells were washed twice in PBS then used for immunostaining and flow cytometry.

We next used Western blotting to investigate the protein translation of VAR4 (SEQ ID NO.: 2) (m10-5). Analysis of PfEMP1 expression of unselected late trophozoite/schizont stage 3D7 using antiserum against the conserved intracellular ATS/Exon2 var domain showed a single band with an estimated molecular weight of 260 kDa (FIG. 17, Lane A). This indicates that most of these parasites expressed a PfEMP1 with a four-domain structure, in correspondence to the results of the absolute quantification (Example 5, Table 2) that showed a dominant group C gene transcript encoding a PfEMP1 molecule of this size. Antibody-selected 3D7 expressed a PfEMP1 species of 260 kDa, but also expressed additional high-molecular weight bands (FIG. 17, Lane B). Again, these data agree with the results of the absolute quantification showing that the three most highly transcribed var genes in antibody-selected 3D7 were PF08_{—}0107, MAL6P1.316, and more specifically PFD1235w/MAL7P1.1 (SEQ ID NO.: 1), which encode proteins with predicted molecular weights of approximately 260, 330, and 400 kDa, respectively. Furthermore, an antiserum against Baculovirus-derived DBL5-δ of the PFD1235w/MAL7P1.1 (SEQ ID NO.: 1) gene product revealed a high-molecular weight band (FIG. 17, Lane D) corresponding to the top band in the ATS/Exon2-probed blot (FIG. 17, Lane B). This indicates that PFD1235w/MAL7P1.1 (SEQ ID NO.: 1) is not only a major transcript in antibody-selected 3D7, but that this var gene is also translated into protein.

Antibodies to Baculovirus-derived DBL5-δ of the PFD1235w/MAL7P1.1 (SEQ ID NO.: 1) gene product were tested on IRBC by flow cytometry, immuno-fluorescence and confocal microscopy (m9-6). Only a small percentage of RBC infected by unselected 3D7 reacted with the murine DBL5-δ antibodies (FIG. 18A1), although these iRBC did express VSA detectable by flow cytometry with human plasma (FIG. 18A3). By contrast, a large proportion of RBC infected with antibody-selected 3D7 were recognized by the murine antibodies, and this reactivity was abrogated by prior trypsin treatment of the iRBC (FIG. 18B1). Fluorescence and confocal microscopy using the DBL5-δ murine antibodies showed a distinct punctate pattern on the surface of intact RBC infected by antibody-selected 3D7 (FIGS. 18B2, 18B5), but not by unselected 3D7 (FIGS. 18A2, 18A5).

Taken together, these results indicate that the product of PFD1235w/MAL7P1.1 (SEQ ID NO.: 1 and 2) is expressed on the surface of antibody-selected 3D7 and that the protein confers a VSA_{SM} phenotype. Similar results were obtained for PF11_{—}0008 (SEQ ID NO.: 3 and 4) and PF13_{—}0003 (SEQ ID NO.: 5 and 6) (data not shown).

### Example 11

### Monoclonal Antibodies to VAR4 (SEQ ID NO.: 2), VAR5 (SEQ ID NO.: 4), VAR6 (SEQ ID NO.: 6)

For production of monoclonal antibodies (mAb) mice were immunized by injection of different domains of VAR4, VAR5, and/or VAR6 to stimulate the production of antibodies targeting these domains.
m11-1. The antibody forming cells were isolated from the spleen of the mice. Monoclonal antibodies were produced by fusing single antibody-forming cells to cancer cells (such as cells from myeloma) to make them immortal. The cells were grown using in vitro cell-culture techniques and cloned by limiting dilution. mAb secreted from the hybridomas were purified and used for identification by flow-cytometry of field P. falciparum isolates expressing VAR4, VAR5, and/or VAR6 or homologous hereof.

This strategy resulted in mAb with different specificities and different avidity and the resulting mAb were used to identify epitopes involved in adhesion to receptors on human endothelial cells and to discriminate between even quite similar epitopes.

### Example 12

### VAR4 (SEQ ID NO.: 2) Recombinant Fusion Proteins are Commonly and Specifically Recognised

If VAR4 (SEQ ID NO 2) mediates a VSA_{SM} phenotype, it would be predicted that a high proportion of children in malaria endemic areas have acquired antibodies to this protein. To test this hypothesis we measured plasma levels of IgG against recombinant domains of the VAR4 protein by ELISA in asymptomatic individuals living under high malaria transmission intensity in Tanzania.

To make recombinant proteins of VAR4 (SEQ ID NO.: 2) the PFD1235w/MAL7P1.1 (SEQ ID NO.: 1) NTS, DBL1-α, C1DR1-α, DBL2-β, C2, DBL3-β, DBL4-γ, DBL5-δ, and CIDR2-β were sub-cloned into the pGEX-4T1 vector by PCR using the following domain-specific oligonucleotide primers and a hot start taq polymerase (Qiagen) and PfuTurbo (Stratagene):

| | |
|---|---|
| NTS.Fw: | 5′-GAATTCATGGGGAATGCATCATCA-3′ |
| NTS.Rv: | 5′-ATAAGAATGCGGCCGCATTTCTATCTTCTGC |
| | GTT-3′ |
| DBL1-α.Fw: | 5′-GAATTCAACGCAGAAGATAGAAATC-3′ |
| DBL1-α..Rv: | 5′-ATAAGAATGCGGCCGCCTTCGATGTATATGT |
| | CTG-3′ |
| CIDR1-α.Fw: | 5′-GAATTCGACGCTAAAACTGATAGTA-3′ |
| CIDR1-α.Rv: | 5′-ATAAGAATGCGGCCGCACATATATCGCCATT |
| | CAACG-3′ |
| DBL2-β.Fw: | 5′-GAATTCTTGAATGGCGATATATGTA-3′ |
| DBL2-β.Rv: | 5′-ATAAGAATGCGGCCGCGTATATAGCTGATAC |
| | TGT-3′ |
| C2.Fw: | 5′-GAATTCACAGTATCAGCTATATACC-3′ |
| C2.Rv: | 5′-ATAAGAATGCGGCCGCATAACATGGTTGTTG |
| | TGA-3′ |
| DBL3-β..Fw: | 5′-GAATTCTCACAACAACCATGTTATG-3′ |
| DBL3-β..Rv: | 5′-ATAAGAATGCGGCCGCATCTATTCCACCTGT |
| | AGT-3′ |
| DBL4-γ.Fw: | 5′-GAATTCACTACAGGTGGAATAGATCA-3′ |
| DBL4-γ.Rv: | 5′-ATAAGAATGCGGCCGCACATGCGGCATTGAG |
| | ACT-3′ |
| DBL5-δ.Fw: | 5′-GAATTCAGTCTCAATGCCGCATGTG-3′ |
| DBL5-δ.Rv: | 5′-ATAAGAATGCGGCCGCTCTACAATGTCTGGC |
| | ACA-3′ |
| CIDR2-β.Fw: | 5′-TCCCCCGGGTGTGCCAGACATTGTAGATC-3′ |
| CIDR2-β.Rv: | 5′-ATAAGAATGCGGCCGCTTTGCCACTAGGTAC |
| | GT-3′ |

The proteins encoding single domains were expressed as fusion proteins (E. coli strain BL21) at the carboxyterminus of glutathione S-transferase from Schistosoma japonicum, and purified by affinity chromatography on glutathione sepharose 4B (Amersham Pharmacia Biotech)

### Materials and Methods

m12-1. We used plasma samples from 20 children (3-4 and 10-11 years of age) and 10 young adults (18-19 years of age) living in Mgome village in the Tanga region of Tanzania for ELISA analysis of antibody responses to purified recombinant NTS, DBL3-β, CIDR1-α and DBL5-δ of VAR4-GST domains as previously described. Briefly, proteins were diluted in 0.1 M glycine/HCl (pH 2.75). The wells of Maxisorp micro titre plates (Nunc, Roskilde, Denmark) were coated with antigen by overnight incubation at 4° C. The plates were emptied, and any residual binding capacity was blocked with 100 μL of blocking buffer (1% bovine serum albumin, 0.5 M NaCl, 1% Triton-X-100 in phosphate-buffered saline (PBS), pH 7.2) per well. After incubation for 0.5 hr at room temperature, the plates were washed four times with washing buffer (PBS, 0.5 M NaCl, 10% Triton-X-100, pH 7.4) and 100 μl of plasma diluted 1:200 in blocking buffer was added to each well. The plates were then incubated for one hour at room temperature, and then washed and incubated for one more hour at room temperature with peroxidase-conjugated rabbit anti-human immunoglobulin G (IgG) (Dako, Glostrup, Denmark) diluted 1:1000 in blocking buffer. Subsequently, the plates were washed and 100 μl of o-phenylenediamine substrate (0.30%, Dako) diluted in 0.1 M sodium citrate buffer (pH 5.0) with 0.05% (v/v) H₂O₂, was added to each well. Finally, the plates were incubated at room temperature in the dark before the addition of 100 μl of 2.5 M H₂SO₄ and the optical density (OD) was measured at 492 nm. Control plates were coated with GST alone. Cut-off values were calculated as the mean ELISA unit plus 2 standard deviations obtained with sera of 13 Danish blood donors without malaria exposure as described in (Jakobsen et al., 1993).
m12-2. Competition ELISA was done using recombinant CIDR1-α domains of VAR4 (SEQ ID NO.: 2) and var1 (PFE1640w) using three different plasma samples of high, medium, and low reactivity with the proteins from two children aged 3 years and one child aged 11 years. Blocking of plasma was done using 0.1, 1, 5, and 10 μg/ml of recombinant protein for 2 hr at room temperature. The test plasma samples were diluted 1:50 and tested as described above.

Most parasite-exposed children and adults had IgG directed against DBL5-δ (FIG. 18A), CIDR1-α (FIG. 18B), DBL3-β (FIG. 18C), and NTS (FIG. 18D), with comparable levels in young children and adults. To test the antigen specificity of the antibody recognition, we performed competition ELISA using the CIDR1-α domains of the VAR4 (SEQ ID NO.: 2) and var1 (PFE1640w) proteins. Pre-incubation of the plasma with homologous recombinant protein caused a dose-dependent reduction in OD₄₉₂ values (FIGS. 18E, 18F), whereas the heterologous protein (FIGS. 18E, 18F) did not change the plasma reactivity.

These result showing that a high proportion of young children recognises recombinant domains of VAR4 and the demonstration that our DBL5-δ antiserum did not cross react with products of MAL6P1.316, and PF08_{—}0107 in Western blotting, suggest that the plasma antibody reactivity to recombinant VAR4 (SEQ ID NO.: 2) protein was the result of exposure to parasites expressing PfEMP1 resembling that encoded by PFD1235w/MAL7P1.1 (SEQ ID NO.: 1), rather than being due to a broad cross-reactivity between different CIDR or DBL domains. Similar results were obtained when using VAR5 (SEQ ID NO.: 4) and VAR6 (SEQ ID NO.: 6) (data not shown).

### Example 13

### Experimental Infection of Humans Results in Transcription and Translation of PFD1235w/MAL7P1.1 (SEQ ID NO.: 1 and 2)

We have proposed that the dominance of parasites expressing VSA_{SM}-type antigens among non-immune patients is related to their higher growth rate in such individuals, and that the shift towards VSA_{UM}-type antigens occurs as this strong selective advantage of VSA_{SM}-expressing parasites gradually disappears as VSA_{SM}-specific immunity is acquired.

### Materials and Methods:

m13-1. We used parasites isolated on days 8, 9, and 10 from a Dutch volunteer exposed on day 0 to mosquitoes infected by P. falciparum isolate NF54 as part of ongoing studies of experimental P. falciparum infections (Hermsen et al., 2001). These parasites were cultured in vitro for 27 (day 8 and day 9 isolates) or 33 days (day 10 isolate) to obtain sufficient parasites for DNA/RNA analysis.
m13-2. SDS-extracted trophozoite/schizont-stage iRBC were reduced by boiling in the presence of L-mercaptoethanol and electrophoresed in the Laemmli sodium dodecyl sulfate polyacrylamide gel electrophoresis (SDS-PAGE) system (Sambrook et al., 1989). Immunoblots were prepared on PVDF membranes by semidry blotting using standard methods. All available binding sites on membranes were blocked in Tris-buffered-saline-Tween (TBS-T) containing 5% skimmed milk. Blots were probed with an antiserum raised against a recombinant protein of the highly conserved intracellular acidic segment ATS/Exon2 conserved among most var genes in 3D7, DBL5-δ of VAR4 (SEQ ID NO.: 2), and pre-immune mouse sera diluted 1:100 in TBS-T. Bound antibody was detected with relevant IgG alkaline phosphatase-conjugated antibody (Dako, Glostrup, Denmark). All washes were done using TBS-T. Blots were developed by standard methods using p-nitroblue tetrazolium phosphate (NBT, Sigma) and 5-bromo-4-chloro-3-indolylphosphate (BCIP, Sigma) as substrates.

To further study this hypothesis we studied parasites rescued on days 8, 9, and 10 from a Dutch volunteer receiving a mosquito-transmitted P. falciparum NF54 infection on day 0. NF54 was originally isolated from a non-immune Dutch malaria patient, and isogenic with the 3D7 parasites cloned from it (Salanti et al., 2003). Western blots of Day 10 parasites probed with antiserum against the DBL5-6 domain of the PFD1235w/MAL7P1.1 gene product, detected a high-molecular weight band (FIG. 17, Lane G), which corresponded in size to that observed in antibody-selected 3D7 (FIG. 17, Lane B). This band was not detected in similar blots of parasites obtained on Day 8 and Day 9 (FIG. 17, Lanes E-F). In line with these findings, we observed high PFD1235w/MAL7P1.1 (SEQ ID NO.: 1) copy numbers relative to those of the seryl tRNA synthetase gene on Day 10, but not on Day 8, corresponding to a 38.30 and 8.4 fold increase in mRNA copies of this var gene relative to the copy number of seryl-tRNA synthetase mRNA between Day 8 and Day 10 in rings and trophozoites, respectively (data not shown). Judged by comparison of Ct-values, ring-stage transcription of PFD1235w/MAL7P1.1 (SEQ ID NO.: 1) was relatively low on Day 8 (rank: 55/60), but high on Day 10 (rank: 9/60). The corresponding rankings in trophozoites were 48 on Day 8 and 18 on Day 10.

Taken together, these data show that PFD1235w/MAL7P1.1 (SEQ ID NO.: 1) is translated into a functional protein in vivo, and indicate that this protein may be associated with fast-growing parasites dominating acute P. falciparum malaria in non-immune patients. Similar results were obtained for PF11_{—}0008 (SEQ ID NO.: 3) and PF13_{—}0003 (SEQ ID NO.: 5) (data not shown).

### Example 14

### Anti-Adhesion Assay

It is becoming increasingly apparent that acquired protective immunity to P. falciparum infection relies on antibodies (Abs) specifically recognizing variant parasite antigens expressed on the surface of late stage-infected erythrocytes. In this scenario, only parasites expressing variant antigens to which the host does not possess adequate specific Ab are likely to cause disease, and immunity is likely to depend on the accumulation of a large panel of Ab specificities recognizing different variants of such antigens. Severe malaria is often associated with sequestration of large quantities of parasites in the brain. Parasites causing severe malaria have been shown to adhere receptors on endothelial cells. To show that VAR4, VAR5, and VAR6 are responsible for in vitro adhesion of 3D7_{SM1} and 3D7_{endo} parasites to endothelial receptors, an antibody adhesion assay with murine antibodies against VAR4, VAR5, and VAR6 were performed.

### Materials and Methods:

m14-1. Antiadhesion was measured by ³H labeled parasites: For use in adhesion assays, parasite cultures with a parasitemia of ˜1% late trophozoites and schizonts were first transferred from Albumax II medium (Life Technologies), with a high concentration of hypoxanthine (Hpx), into RPMI 1640 plus 5% normal human serum (low Hpx) and maintained for 24 h. The parasites then were labeled by exposure to [3H]Hpx (Amersham; 8.75 MBq/mL of RBCs) for another 24 h. Finally, the cultures were enriched for late-stage iRBCs and incubated for 30 min, with or without test plasma. Endothelial cells were grown on Microtiter plates (Falcon; Becton Dickinson) then blocked with bovine serum albumin (BSA; 20 mg/mL, 100 μL/well) in PBS at room temperature for 30 min. We added enriched [³H]Hpx-labeled late-stage iRBCs to wells containing endothelial cells (2×10⁶ cells/well) and incubated the wells at 37° C. for 1 h. Nonadherent iRBCs were removed by 4 washes in RPMI 1640. Adherent iRBCs were harvested onto glass fiber pads, and the [³H]Hpx activity was measured in a liquid scintillation counter (Beckman Coulter). Inhibition of iRBC adhesion by plasma was calculated as 1−(testEndothelialCell−controlBSA)/controlEndothelialCell−controlBSA), where testEndothelial is counts per minute of iRBCs preincubated with plasma and adhering to wells containing endothelial cells, and controlEnothelial cells and controlBSA refer to counts per minute of iRBCs not preincubated with plasma and adhering to endothelial cells and BSA-coated wells, respectively.

Cytoadhesion of endothelial cells were significantly inhibited by plasma from individuals suffering from severe malaria, and more importantly binding of 3D7_{SM1} and 3D7_{endo} to endothelial cells were strongly inhibited by the murine anti-VAR4, -VAR5, and -VAR6 antibodiesAntibodies raised against recombinant VAR4, -VAR5, and -VAR6 inhibit parasite adhesion to endothelial cells in vitro (data not shown).

An obvious consequence of this finding is that vaccine induced antibodies against SEQ ID NO.: 2, 4, and/or 6 constructs can hinder binding of parasites to endothelial tissue and thus prevent severe malaria.

### Example 15

### Identification of Receptors and VAR4 (SEQ ID NO.: 2), VAR5 (SEQ ID NO.: 4), and/or VAR6 (SEQ ID NO.: 6) Sites Binding Receptors on Endothelial Cells in vitro

To further study endothelial cell adhesion all domains were cloned into the pDISPLAY vector (Invitrogen) using the following primers as exemplified for VAR4:

| | |
|---|---|
| NTS.Fw: | 5′-TCCCCCGGGATGGGGAATGCATCATCA-3′ |
| NTS.Rv: | 5′-TCCCCGCGGATTTCTATCTTCTGCGTT-3′ |
| DBL1-α.Fw: | 5′-TCCCCCGGGAACGCAGAAGATAGAAATC-3′ |
| DBL1-α..Rv: | 5′-TCCCCGCGGCTTCGATGTATATGTCT-3′ |
| CIDR1-α.Fw: | 5′-TCCCCCGGGGACGCTAAAACTGATAGTA-3′ |
| CIDR1-α.Rv: | 5′-TCCCCGCGGACATATATCGCCATTCAACG-3′ |
| DBL2-β.Fw: | 5′-TCCCCCGGGTTGAATGGCGATATATGTA-3′ |
| DBL2-β.Rv: | 5′-TCCCCGCGGGTATATAGCTGATACTGT-3′ |
| C2.Fw: | 5′-TCCCCCGGGACAGTATCAGCTATATACC-3′ |
| C2.Rv: | 5′-TCCCCGCGGATAACATGGTTGTTGTGA-3′ |
| DBL3-β..Fw: | 5′-TCCCCCGGGTCACAACAACCATGTTATG-3′ |
| DBL3-β..Rv: | 5′-TCCCCGCGGATCTATTCCACCTGTAGT-3′ |
| DBL4-γ.Fw: | 5′-TCCCCCGGGACTACAGGTGGAATAGATCA-3′ |
| DBL4-γ.Rv: | 5′-TCCCCGCGGACATGCGGCATTGAGACT-3′ |
| DBL5-δ.Fw: | 5′-TCCCCCGGGAGTCTCAATGCCGCATGTG-3′ |
| DBL5-δ.Rv: | 5′-TCCCCGCGGTCTACAATGTCTGGCACA-3′ |
| CIDR2-β.Fw: | 5′-TCCCCCGGGTGTGCCAGACATTGTAGATC-3′ |
| CIDR2-β.Rv: | 5′-TCCCCGCGGTTTGCCACTAGGTACGT-3′ |

The ability of the different domains to bind directly to endothelial cells was in this example assayed using a mammalian expression system.
m15-1. Domains were cloned into the pDisplay vector (Invitrogen). This vector allows display of cloned proteins on the cell surface. Each domain was fused at the N-terminus to the murine Ig K-chain leader sequence, which targets the protein to the cell surface, and at the C-terminus to the platelet derived growth factor receptor (PDGFR) transmembrane domain, which anchors the protein to the cell membrane.

A human non-adherent T cell and a CHO cell line was used for transient expression of the recombinant proteins. This approach has enabled us to study cell adhesion to endothelial cells. Blockage of this binding was studied using relevant recombinant domains expressed in the Baculovirus system (Example 10) and commercial antibodies directed against specific endothelial receptors.

VAR4 (SEQ ID NO.: 2), VAR5 (SEQ ID NO.: 4), and/or VAR6 (SEQ ID NO.: 6) regions responsible for receptor binding were identified using random mutagenesis.

### Example 16

To express VAR4 (SEQ ID NO.: 2), VAR5 (SEQ ID NO.: 4), and/or VAR6 (SEQ ID NO.: 6) in eucaryotic organisms the Exon 1 ranging from nt 1 to 9444 (SEQ ID NO.: 1), 7719 (SEQ ID NO.: 3), and 8844 (SEQ ID NO.: 5) were subjected to a full recodonisation:

An artificial codon table was generated by combining the codon usage of Trichoplusia ni and Homo sapiens genes. The codon bias of the synthetic genes were adapted to this “artificial” codon usage. In addition, regions of very high (>80%) or very low (<30%) GC content was avoided and the GC-content was adjusted to 50% where possible. During the optimization process following cis-acting sequence motifs were avoided: internal TATA-boxes, chi-sites and ribosomal entry sites, AT-rich or GC-rich sequence stretches, repeat sequences and RNA secondary structures, and (cryptic) splice donor and acceptor sites, branch points

No reverse-complementary sequence identities longer than 20 nucleotides are found when the optimized sequence is aligned to the transcription of Homo sapiens. No RNA interference should therefore be expected. The entire gene was divided into and constructed as four ˜2 kb long fragments using PstI (2028), KasI (3759) and PvuII (5899) and cloned into pCR-Script-Amp (Stratagene, Calif., USA) KpnI and SacI restriction sites.

The recodonised PFD1235w/MAL7P1.1, PF11_{—}0008, and PF13_{—}0003 NTS, DBL1-α, CIDR1-α, C1DR1-γ, DBL2-β, DBL2-γ, C2, DBL3-β, DBL3-γ, DBL4-γ, DBL4-δ, DBL4-β, DBL5-δ, DBL5-β, and CIDR2β were expressed in Baculovirus infected hi-five insect cells and purified by HIS tag Metal Chelate Affinity Chromatography purification by cloning the domains into the pAcGP307-A (BD Biosciences, Brøndby, Denmark) and Eucaryotic TA expression vector pCR3.1 (Invitrogen).

### Example 17

### Field Isolates of P. falciparum Causing Severe Malaria Shows High Transcription of PFD1235w/MAL7P1.1 (SEQ ID NO. 1), PF11_{—}0008 (SEQ ID NO.: 3), and/or PF13_{—}0003 (SEQ ID NO.: 5) or Homologues Hereof

From Example 3, 4, 5, and 6 it appears that the Group A PFD1235w/MAL7P1.1 (SEQ ID NO. 1), PF11_{—}0008 (SEQ ID NO.: 3), and/or PF13_{—}0003 (SEQ ID 5) gene encodes VSA_{SM}-like proteins, and that these proteins are involved in the acquisition of the VSA_{SM} phenotype following antibody-selection of VSA_{UM}-expressing 3D7 (3D7_{SM}) and adhesion to endothelial cells (3D7_{endo}).

### Materials and Methods

m17.1. Real-time PCR was done using a Rotorgene thermal cycler system (http://www.corbettresearch.com) following purification of RNA and synthesis of cDNA (m5.1). The transcription level of PFD1235w/MAL7P1.1 (SEQ ID NO. 1), PF11_{—}0008 (SEQ ID NO.: 3), and PF13_{—}0003 (SEQ ID NO.: 5) or homologues of hereof were measured using primers targeting this sequence. The absolute copy number was calculated (m5.3) and compared to the absolute copy number of other var genes present in the field isolates as well as housekeeping genes such as seryl-tRNA synthetase. Reactions were performed as in (m5.2)

The transcription of PFD1235w/MAL7P1.1 (SEQ ID NO. 1), PF11_{—}0008 (SEQ ID NO.: 3), and/or PF13_{—}0003 (SEQ ID NO.: 5) or homologues hereof were found to be higher than other var genes in parasites causing severe malaria, but low in parasites causing uncomplicated malaria (data not shown).

### Example 18

### Plasmodium Falciparum Field Isolates Causing Severe Malaria Expresses VAR4 (SEQ ID NO.: 2) on the Surface of Infected Erythrocytes

Analysis of VAR4 (SEQ ID NO.: 2) expression of RBC infected with parasites causing severe malaria using antibodies to Baculovirus-derived DBL5-δ of the PFD1235w/MAL7P1.1 gene product revealed a high-molecular weight band of approximately 400 kDA corresponding to the size of the VAR4 expressed by 3D7_{SM} (Example 5), which was absent in parasites with a VSA_{UM} phenotype (data not shown).

Taken together, the results of Example 17 and 18 indicates that PFD1235w/MAL7P1.1 (SEQ ID NO.: 1) or homologues hereof are not only transcribed, but also expressed on the surface of RBC infected with parasites having a VSA_{SM} phenotype and causing severe malaria, but not parasites causing uncomplicated malaria and having a VSA_{UM} phenotype. Similar results were obtained for PF11_{—}0008 (SEQ ID NO.: 3), and PF13_{—}0003 (SEQ ID NO.: 5) (data not shown).

### Example 19

### Plasmodium Falciparum Field Isolates Causing Severe Malaria Adhere to Endothelial Cells and Adhesion is Inhibited by SEQ ID NO. 2, 4, and/or 6 Specific Antibodies

To show that VAR4, VAR5, and/or VAR6 are responsible for in vitro adhesion of field isolates to endothelial receptors, an antibody adhesion assay with murine antibodies against VAR4, VAR5, and VAR6 were performed.

Materials and methods:
m19-1. Antiadhesion was measured by ³H labeled parasites: For use in adhesion assays, parasite cultures with a parasitemia of ˜1% late trophozoites and schizonts were first transferred from Albumax II medium (Life Technologies), with a high concentration of hypoxanthine (Hpx), into RPMI 1640 plus 5% normal human serum (low Hpx) and maintained for 24 h. The parasites then were labeled by exposure to [3H]Hpx (Amersham; 8.75 MBq/mL of RBCs) for another 24 h. Finally, the cultures were enriched for late-stage iRBCs and incubated for 30 min, with or without test plasma. Endothelial cells were grown on Microtiter plates (Falcon; Becton Dickinson) then blocked with bovine serum albumin (BSA; 20 mg/mL, 100 μL/well) in PBS at room temperature for 30 min. We added enriched [³H]Hpx-labeled late-stage iRBCs to wells containing endothelial cells (2×10⁶ cells/well) and incubated the wells at 37° C. for 1 h. Nonadherent iRBCs were removed by 4 washes in RPMI 1640. Adherent iRBCs were harvested onto glass fiber pads, and the [³H]Hpx activity was measured in a liquid scintillation counter (Beckman Coulter). Inhibition of iRBC adhesion by plasma was calculated as 1−(testEndothelialCell−controlBSA)/controlEndothelialCell−controlBSA), where testEndothelial is counts per minute of iRBCs preincubated with plasma and adhering to wells containing endothelial cells, and controlEnothelial cells and controlBSA refer to counts per minute of iRBCs not preincubated with plasma and adhering to endothelial cells and BSA-coated wells, respectively.

Cytoadhesion to endothelial cells were significantly inhibited by plasma from young semi-immune children, and more importantly binding of field isolates to endothelial cells were strongly inhibited by the murine anti-VAR4, -VAR5, and -VAR6 antibodies. In this example it is shown that antibodies raised against recombinant VAR4, -VAR5, and -VAR6 inhibit field parasite isolate adhesion to endothelial cells in vitro.

An obvious consequence of this finding is that vaccine induced antibodies against SEQ ID NO.: 2, 4, and/or 6 constructs can hinder binding of parasites to endothelial tissue and thus prevent severe malaria.

### Example 20

### A Novel DynaBead Based Method and Adhesion to Endothelial Cells Selects for RBC Infected with Parasites Having a VSA_{SM} Phenotype and which Transcribes Var Genes with Similar Characteristics

Selection of parasites using a novel antibody-DynaBead based method (Example 3) and adhesion to endothelial cells (Example 6) leads to changes in the serological phenotype of the 3D7 parasites from being of a VSA_{UM}- to a VSA_{SM}-type (Example 3 and FIGS. 5C-D). This VSA_{SM} phenotype resembles that of parasites causing severe malaria (Example 1 and FIG. 1). Thus, this method and results provided by it forms the basis for identification of PfEMP1 molecules that could be used as part of a vaccine against severe malaria.

Using this method we were able to identify three different var genes PFD1235w/MAL7P1.1 (SEQ ID NO.: 1), PF11_{—}0008 (SEQ ID NO.: 3), and PF13_{—}0003 (SEQ ID NO.: 5) that were highly transcribed and upregulated in 3D7_{SM} (FIGS. 13, 14, and 20, Example 5 and 6, Table 2). The protein products of these three genes are responsible for the serological phenotype and adhesive properties of 3D7_{SM}. Interestingly, these three genes do not belong to the var1, var2 gene subfamily as defined previously (Salanti et al. 2002, Salanti et al. 2003) or Group B, Group C, and Group B/C var genes as defined recently (Lavstsen et al. 2003). They lack 1-2 cysteine residues in DBLα homology group G (Smith et al. 2000) compared to most PfEMP1 molecules in Group B, C, and B/C. Additionally, they posses a CIDR1α distinctly different from the CIDR1α domain of the MCvar1 PfEMP1 gene product expressed by Malayan Camp parasites and of the A4VAR expressed by A4 parasites that have been shown to bind CD36 (Baruch et al. 1997; Cooke et al. 1998; Bryan et al. 2003; Smith et al. 1998). By contrast, 3D7_{SM} that transcribes PFD1235w/MAL7P1.1 (SEQ ID NO.: 1), PF11_{—}0008 (SEQ ID NO.: 3), and/or PF13_{—}0003 (SEQ ID NO.: 5) do not bind to CD36 (Example 3).

In conclusion, the three genes are present and expressed in field isolates causing severe malaria, but not in parasites with a VSA_{UM} phenotype (Example 17, 18, and 19) indicating that the serve similar functions and might be responsible for the pathogenesis of severe malaria.

### REFERENCES

Baruch D I, Ma X C, Singh H B, Bi X, Pasloske B L, Howard R J. (1997). Identification of a region of PfEMP1 that mediates adherence of Plasmodium falciparum infected erythrocytes to CD36: conserved function with variant sequence. Blood 90, 3766-3775
Bian Z, Wang G, Tian X and Fan J. (1999). Expression of Plasmodium falciparum-infected erythrocyte membrane protein from cerebral malaria patients. Zhongguo Ji Sheng Chong Xue Yu Ji Sheng Chong 17, 359-362.
Bian, Z. Q. and Wang, G. Z. (2000). Antigenic variation and cytoadherence of PfEMP1 of Plasmodium falciparum-infected erythrocyte from malaria patients. Chinese Medical Journal 113, 981-984.
Bryan Y G., Dror B I, Claran B, Allan M G., Sornchai L, Kubes, P., and Ho, M. (2003). Recombinant PfEMP1 peptide inhibits and reverses cytoadherence of clinical Plasmodium falciparum isolates in vivo. Blood 101, 331-337.
Cooke B M, Nicoll C L, Baruch D I, Coppel R L. (1998). A recombinant peptide based on PfEMP-1 blocks and reverses adhesion of malaria-infected red blood cells to CD36 under flow. Mol. Microbiol. 30, 83-90
Delemarre B J & Van der Kaay H J. (1979). Malaria tropica op natuurlijke wijze verkregen in Nederland. Ned Tijdschr Geneeskd 123, 1981-1982.
Dodoo, D., Staalsoe, T., Giha, H., Kurtzhals, J. A., Akanmori, B. D., Koram, K., Dunyo, S., Nkrumah, F. K., Hviid, L., and Theander, T. G. (2001). Antibodies to variant antigens on the surfaces of infected erythrocytes are associated with protection from malaria in Ghanaian children. Infect. Immun. 309, 3713-3718.
Fernandez, V., Hommel, M., Chen, Q., Hagblom, P., Wahigren, M. (1999). Small, clonally variant antigens expressed on the surface of the Plasmodium falciparum-infected erythrocyte are encoded by the rif gene family and are the target of human immune responses. J. Exp. Med. 190, 1393-1403.
Gardner M I, Hall N, Fung E, White O, Berriman M, Hyman R W, Carlton J M, Pain A, Nelson K E, Bowman S, Paulsen I T, James K, Eisen J A, Rutherford K, Salzberg S L, Craig A, Kyes S, Chan M S, Nene V, Shallom S J, Suh B, Peterson J, Angiuoli S, Pertea M, Allen J, Selengut J, Haft D, Mather M W, Vaidya A B, Martin D M A, Fairlamb A H, Fraunholz M I, Roos D S, Ralph S A, Mcfadden G I, Cummings L M, Subramanian G M, Mungall C, Venter I C, Carucci D I, Hoffman S L, Newbold C, Davis R W, Fraser C M, Barrell B. (2002) Genome sequence of the human malaria parasite Plasmodium falciparum. Nature 419, 498-511.
Gupta, S., Snow, R., Donnelly, C., Marsh, K., and Newbold, C. (1999). Immunity to non-cerebral malaria is acquired after one or two infections. Nature Medicin. 5, 340-343.
Hall T A: (1999). BioEdit: a user-friendly biological sequence alignment editor and analysis program for Windows 95/98/NT. Nucl Acids Symp Ser 41, 95-98.
Hasler T, Albrecht G R, Van Schravendijk M R et al. (1993). An improved microassay for Plasmodium falciparum cytoadherence using stable transformants of Chinese hamster ovary cells expressing CD36 or intercellular adhesion molecule-1. Am J Trop Med Hyg 48, 332-347.
Hermsen, C. C., Telgt, D. S. C., Linders, E. H. P., Van de Locht, L. A. T. F., Eling, W. M. C., Mensink, E. J. B. M., and Sauerwein, R. W. (2001). Detection of Plasmodium falciparum malaria parasites in vivo by real-time quantitative PCR. Mol. Biochem. Parasitol. 118, 247-251.
Howard, R. J. and Barnwell, J. W. (1984). Roles of surface antigens on malaria-infected red blood cells in evasion of immunity. Contemp. Top. Immunobiol. 12, 127-191.
Kumar S, Tamura K, Jakobsen I and Nei M. (2001). MEGA2: Molecular Evolutionary Genetics Analysis. Ver. 2.1. Bioinformatics 17, 1244-1245.
Jakobsen, P. H., Hviid, L., Theander, T. G., Afare, E. A., Ridley, R. G., Heegard, P. M. H., Stuber, D., Dalsgaard, K., and Nkrumah, F. K. (1993). Specific T-cell recognition of the merozoite proteins rhoptry-associated protein 1 and erythrocyte-binding antigen 1 of Plasmodium falciparum. Infect. Immun. 61, 268-273.
Kraemer S M, Smith J D. (2003). Evidence for the importance of genetic structuring to the structural and functional specialization of the Plasmodium falciparum var gene family. Mol. Microbiol. 50, 1527-38.
Kirchgatter, K. and Del Portillo, H. A. (2002). Association of severe non-cerebral Plasmodium falciparum malaria in Brazil with expressed PfEMP1 DBL1α sequences lacking cysteine residues. Mol. Med. 8, 16-23.
Lavstsen, T., Salanti, A., Jensen, A. T. R., Arnot, D. E., and Theander, T. G. (2003). Sub-grouping of Plasmodium falciparum 3D7 var genes based on sequence analysis of coding and non-coding regions. Malar. J. 2.
McGregor, I. A., Carrington, S. P., and Cohen, S. (19303). Treatment of East African P. falciparum malaria with West African human g-globulin. Trans. R. Soc. Trop. Med. Hyg. 57, 170-175.
Mercereau-Puijalon O, Barale J C, Bischoff E. (2002). Three multigene families in Plasmodium parasites: facts and questions. Int J Parasitol. 32, 1323-1344.
Newbold, C. I., Craig, A. G., Kyes, S., Berendt, A. R., Snow, R. W., Peshu, N., and Marsh, K. (1997). PfEMP1, polymorphism and pathogenesis. Ann. Trop. Med. Parasitol. 91, 551-557.
Nielsen, M. A., Staalsoe, T., Kurtzhals, J. A., Goka, B. Q., Dodoo, D., Alifrangis, M., Theander, T. G., Akanmori, B. D., and Hviid, L. (2002). Plasmodium falciparum variant surface antigen expression varies between isolates causing severe and nonsevere malaria and is modified by acquired immunity. J. Immunol. 1308, 3444-3450.
Riley, E. M., L. Hviid, T. G. Theander. (1994). Malaria. F. Kierszenbaum, ed. Parasitic Infections and the Immune System 119. Academic, New York.
Robinson, B. A., Welch, T. L., and Smith, J. D. (2003). Widespread functional specialization of Plasmodium falciparum erythrocyte membrane protein 1 family members to bind CD330 analysed across a parasite genome. Mol. Microbiol. 47, 12305-1278.
Rogerson S I, Chaiyaroj S C, Ng K, Reeder I C & Brown G V. (1995). Chondroitin sulfate A is a cell surface receptor for Plasmodium falciparum-infected erythrocytes. J Exp Med. 182, 15-20.
Salanti A, Jensen A T R, Zornig H D, Staalsoe T, Joergensen L, Nielsen M A, Khattab A, Arnot D E, Klinkert M Q, Hviid L and Theander T G. (2002). A sub-family of common and highly conserved Plasmodium falciparum var genes. Mol Biochem Parasitol 122, 111-115.
Salanti, A., Staalsoe, T., Lavstsen, T., Jensen, A. T., Sowa, M. P., Arnot, D. E., Hviid, L., and Theander, T. G. (2003). Selective upregulation of a single distinctly structured var gene in chondroitin sulphate A-adhering Plasmodium falciparum involved in pregnancy-associated malaria. Mol Microbiol 2003. Jul.; 49. (1):179.-91. 49, 179-191.
Sambrook, J., Fritsch, E. F., and Maniatis, T. (1989). Molecular cloning: a laboratory manual. (Cold Spring Harbor: Cold Spring Harbor Laboratory Press).
Silamut, K., Phu, N. H., Whitty, C., Turner, G. D. H., Louwrier, K., Mai, N. T. H., Simpson, J. A., Hien, T. T., and White, N. J. (1999). A quantitative analysis of the microvascular sequestration of malaria parasites in the human brain. Am. J. Pathol. 155, 395-410.
Smith J D, Kyes S, Craig A G, Fagan T, Hudson-Taylor D, Miller L H, Baruch D I, Newbold C I. (1998). Analysis of adhesive domains from the A4VAR Plasmodium falciparum erythrocyte membrane protein-1 identifies a CD36 binding domain. Mol Biochem Parasitol. 97, 133-48.
Smith, J. D., Subramanian, G., Gamain, B., Baruch, D. I., and Miller, L. H. (2000). Classification of adhesive domains in the Plasmodium falciparum erythrocyte membrane protein 1 family. Mol. Biochem. Parasitol. 2000. Oct.; 110. (2):293.
Staalsoe T, Giha H A, Dodoo D, Theander T G and Hviid L. (1999) Detection of antibodies to variant antigens on Plasmodium falciparum infected erythrocytes by flow cytometry. Cytometry 35, 329-336.
Staalsoe T, Nielsen M A, Vestergaard L S, Jensen A T R, Theander T G and Hviid L. (2003). In vitro selection of Plasmodium falciparum 3D7 for expression of variant surface antigens associated with severe malaria in African children. Parasite Immunol 25, 421-427.
Tatusova T A and Madden T L. (1999). FEMS Microbiol Lett. 174, 247-50.
Wahlgren, M., Fernandez, V., Chen, Q., Svard, S., and Hagblom, P. (1999). Waves of malarial variations. Cell 96, 3003-3006.

1. (canceled)

59. An immunogenic composition comprising an amount of an isolated polypeptide effective for inducing an immune response against Plasmodium falciparum parasites in a mammalian subject and a pharmaceutically acceptable carrier, wherein the isolated polypeptide comprises at least one amino acid sequence selected from the group consisting of:
(a) SEQ ID NO:2, SEQ ID NO:4, and SEQ ID NO:6;
(b) a sequence having at least 80% sequence identity to SEQ ID NO:2, SEQ ID NO:4 or SEQ ID NO:6;
(c) a sub-sequence of the sequences in (a) or (b), wherein the sub-sequence has a minimum length of 10 amino acids; and
(d) a sub-sequence of the sequences in (a) or (b), wherein the sub-sequence comprises at least one B-cell epitope or at least one T-cell epitope.

60. A method for inducing an immune response against Plasmodium falciparum parasites in a mammalian subject, comprising administering to a mammalian subject an amount of an immunogenic composition effective to induce an immune response against Plasmodium falciparum parasites in the mammalian subject, wherein the immunogenic composition comprises an isolated polypeptide which comprises at least one amino acid sequence selected from the group consisting of:
(a) SEQ ID NO:2, SEQ ID NO:4, and SEQ ID NO:6;
(b) a sequence having at least 80% sequence identity to SEQ ID NO:2, SEQ ID NO:4 or SEQ ID NO:6;
(c) a sub-sequence of the sequences in (a) or (b), wherein the sub-sequence has a minimum length of 10 amino acids; and
(d) a sub-sequence of the sequences in (a) or (b), wherein the sub-sequence comprises at least one B-cell epitope or at least one T-cell epitope.

61. The methods of claim 60, wherein the immune response prophylactically or therapeutically reduces the incidence, prevalence or severity of malaria in the mammalian subject.

62. A method for prophylactically or therapeutically reducing the incidence, prevalence or severity of malaria in a mammalian subject, comprising administering to a mammalian subject an amount of an immunogenic composition effective to prophylactically or therapeutically reduce the incidence, prevalence or severity of malaria in the mammalian subject, wherein the immunogenic composition comprises an isolated polypeptide which comprises at least one amino acid sequence selected from the group consisting of:
(a) SEQ ID NO:2, SEQ ID NO:4, and SEQ ID NO:6;
(b) a sequence having at least 80% sequence identity to SEQ ID NO:2, SEQ ID NO:4 or SEQ ID NO:6;
(c) a sub-sequence of the sequences in (a) or (b), wherein the sub-sequence has a minimum length of 10 amino acids; and
(d) a sub-sequence of the sequences in (a) or (b), wherein the sub-sequence comprises at least one B-cell epitope or at least one T-cell epitope.


SEQUENCE LISTING (6 sequences)

SEQ ID NO: 1 - DNA, 10662 bp, Plasmodium falciparum
atggggaatgcatcatcatcagagggggaggctaaaacccctagtttaacagaaagtcacaacagtgcaagaaatattttggaaggttatgccgaaagtataaaggaacaggcatcaaaagatgcaaaaatacatggacatcatttgaaaggagatttggcgaaagcagtatttcgtcatccattttctgcatatagacctaactatggaaatccgtgcgaacttgattataggtttcatactaatgtatggcatcgtaacgcagaagatagaaatccttgtctttttagtcgtgcaaaacgtttttcaaatgaaggtgaagcagaatgtaatggtggtataataactggtaataaaggtgaatgtggggcatgtgcaccgtataggagaagacatatatgtgactataatttgcaccatataaacgaaaataatataaggaatactcatgatttattggggaatttgttagttatggcaaggagtgaaggtgaatctattgtgaaaagtcatgaatatacaggttatggtatatacaaatcaggtatatgtacttctcttgctcgcagttttgcagatataggagatattattcgaggaaaagatctgtatcgtcgtgatagtagaacagataaattagaagagaatttaagaaaaattttcgcgaatatatataaagaattgaagaatgggaagaagtgggcggaagcaaaagagtactaccaagatgatggaactggaaattattataaattaagggaagcttggtgggcacttaacagaaaagatgtgtggaaagcattaacatgtagtgcgccaagggatgctcaatatttcataaaatcaagcgtcagggatcaaacattttcaaatgattattgtggccatggtgaacatgaggttcttacaaatttagattatgtccctcaatttttacgatggtttgaagaatgggcagaagagttttgtagaataaaaaaaataaaattaggaaaggttaaggaagcatgtcgtgatgactcaaaaaaattatattgtagtcataatggatatgactgtacgaaaactattcgaaataaagatattttgtctgataatcctaaatgtactgggtgttctgttaaatgcaaagtttatgaactttggttaaggaatcaacgaaatgaatttgaaaaacaaaaaaaaaaatattataaggaaatacagacatatacatcgaaggacgctaaaactgatagtaatattaataacgaatattataaggaattttatgacaaacttaaaaatgagggctatgaaacattgaacaaatttataaaattactaaatgaaggaaggtattgtaaagaaaaaatatcaggagaaaggaatattgattttactatgactggtgataaagacgcgttttatcgctcagactattgccaaatatgtcctgaatgtggagtccaatgtagcggtacaacatgcacaccaaaaaaagtgatacatccgaattgcaaagataaagaaacttatgagcctggtgatgcaaaaaccactgatattactgtcctttatagtggtgatgaagaaggtgatattgcacaaaaattacaagatttttgtaatgataaaaataaagaaaatgatgaaaactatgaaaaatggcaatgctattataaaagtagtgagattaataaatgtcaaatgacaccatcatcacacaaagttccaaaacatggttacattatgtcattttatgctttttttgatttgtgggttaagaatttattaatagatagtataaattggaagaacgatcttacgaattgtataaataatactaatgttacggattgtaaaaatgattgtaacacaaattgtaaatgttttgaaaattgggctaaaacaaaagaaaatgagtggaaaaaagtgaagacgatatacaaaaatgaaaacggaaacacgaacaattattataaaaaacttaataaccattttcaaggttatttttttcacgttatgaaagagcttaacaaagaagaaaaatggtataaacttatggaagatttaaaagaaaaaattgattcttccaatttgaaaaatggtacgaaagattcagaaggcgcaataaaagtgttgttcgatcacttaaaagatatagctgaaagatgcatagacaataattcaaaagattcatgtccaccttcagtggatacgaaaacaaacccctgtgctaaacctcctggtagtaaacccactaaaagtgtaaaacaattagcggaacatatgcaacagaaggcacaaaaacttttgggtactcgtggtggtgaaagtaaattgaagggggatgcaacaagagggacgtataaccttggaggtcaaggaaacacgttgaatggcgatatatgtaaaataacaaaaaatcataccaatgatagtcgtcctaatggtgaaccatgtacaggtaaagataaagttaaaaacgggtttcgcttgaaaataggaaccccgtggacaaatattgtacaaaaaaaaaaaaaaaagtcatacaaagacttctatttacctcctcggcgtcaacatatgtgtacatcaaatttagaaaatttgagcacgagtagcaaaggacttagtaatggtagttttgctagtcactcattattaggtgatgtattgctcgcagcaaaatttgaagcacaaaagataatactagtgtataaaaataagaataatataaatatcagaaaaagaataactgacccaaatgatcaagctactgtatgtcgtgctatacgttacagtttcgccgatctaggagatattatacgaggaaaagacatgtggaatataaacagtgatgcaaaagatcttcaagatcgtttagaaaaaatatttaaaaccattaatgaaaaacttcctaatgaaatccaaaaaagatatacgaaccgtgaaaataaacatttagatttacgttcagactggtgggaagctaatagacatcaagtttggagagctatgaaatgtgcaacaaaaggcatcagcaataacaattgtaatggtatcccaatagaagattacatcccacaacgattaagatggatgacggaatgggccgaatggtattgcaaaaagcagtcacaggaatatgagaagttggaggagaagtgtgggatgtgtacgggtaagggtcagggtgatggtaaagattgtacacagaaggataaagaatgtagtccgtgcaagaaagcatgtgatgcatataagaaggaaatagaaaaatgggaaaaacaatggaaaacagtatcagctatataccaaatattatacgcaaaagcacgaattgttgctagtaatggcggtcctgggtattataatacggaagtacagaagaaagaccgatccgtgtatgacttcttgtacgagttacatttacaaaatggtggcaaaaaaggtcctcctcctgctacacatccttataaatctgttaacacacgtgataaacgtgatgccactgatgatacaacacccactgtgtatagtactgctgcaggatatgtacaccaagaagcacatattggtgattgtaaggaacaacacgttttttgtgataataacggcaacaaggagaagtatgcttttaagaatccaccaaatgtatatgttgaggcgtgtaagtgtatgacgagggaggcaccaccaccaccaacaactccttctactccaaatccgtgtgctgaaactggtggtgtacataccattaaaactgtgactgatgtcgcaaaaatattacagggggaggcaaatgaaacaatgctaaaaaatagttccaatggtaatgataaggatgagagtaaattgaaaggtaaggcagaagaaggggattatagtcgtggaggtacgccaagtgacttcaacaacaatttatgtggtataacacaaaagcattccaatgctcataatgattcacaacaaccatgttatggaaaagatcaaaaaaggttcaatgtaggaacggaatggtcatttaaggataatcatagaaaacggacacaccctgaggcatatatgcctccaagaagggaacatatatgtacatcaaatttggaatatttaattcataagagaaaaaaaccaattatagaaggtgatcctaacaagattattcattccttattgggcgatgtgttacttgcagcaaaatatgaagcagaaaacataaagaaactgtatgaagaaaataacaaccgaaaagatcaggaaggtatatgtcgagctatgaaatatagttttgcagatataggggatattattcgaggaaaagatatgtggatagaaaacaatgatgctaagagattacaaacaaatttgaaagaaatatttactaaaattaaagaaaaaactggaggcaccacatataatgaagataacgatccgtatttaaaattacgtgcagattggtgggaagctaatagagccaaagtatggaaagcaatgaaatgtaaaacaaatggcgtagatatcacttgtgatagtgatcatacaccattggatgattatatcccccaaagattaagatggatgactgaatgggcagaatggtattgtaaagcgcaatcacaggagtataagaagttggaggagaagtgtagtcaatgcaagagtaagggtaaaggtgggaatgagtgttacagagaaacgaaggaatgtaacgattgcaagcaagcatgtgaagaatataaaaggaaaataaaaacatgggcagatcaatggaaggtaatatcaaataaatacgaggatttatacaaaaaagcccaaaatcctactaatgctgttctcaaagataacaaagatgagaaagacaagaatgtgattgattttttgacacaattacaaaaagcaaataatggcgaaaaaactggtgtgcacaccgtgtattctactgctgcaggatatatacatcaagaggcacgtacacgtgaatgccaggaacaaagggagttttgtgataaaaaaaatggcattgacaacacaagttatgcttttaaggatccacctcatgggtatgctacagcgtgtgattgcataaataggtcgcaaacagaggagccgaagaaaaaggaagaaaatgtagagagtgcgtgcaaaatagtggaagaagttctttcgaaaccacgagataaaactacaggtggaatagatcattgtaatccaaagtattatccaagaaaagaaaattatcctggatggaattgtactccaggtcagtttaaatcaggtcatgctggagcatgtatgcctccaagaagaataaaattatgtgtaattaatttacaatatttgaatgagaagaaatcaccagaagaattgagaaaagcttttattcaatgcgctgcaatagaaacgtattggttatggcaaaaatacaaaaaggataagaatggtggtgttgcacaagcaaaattaaatagtggtaccatccctgatgactttaagcgtcaaatgttctatacgtttggagattatagagatttatgtttagatactgatatatcatcaaaagcagatacaagtacaggtgtaggtaaagtaaaaattaatatagattctgttttccaaaaaattgacataactaatgtcgaacaacgtaaaccttggtggggaaaaaacgcagaagctatttgggatggaatgttatgtgctttaagttataatactacaaacaaaaatatggattacaatgcacacacaaaattaaatcccacgtacggctacaacgccataaaatctgaactggaagactttgtgaacagacctcaattccttcgatggttcactgaatggagtgacgaattttgtacagaacgtagtataaagatcaaggagttggaaacaaaatgtaacgattgtactgttagtgagagtggtactagtgatgctacgggtaataaaacatgtgatgataaagataaatgtgacgagtgcaaaagagcatgtacaacatataaaacttggcttaaaaattggaaaactcaatataaaacacaaagcaaaaaatattttgatgataaaagaaaagaactatataaaagtatcgatgacgtcgccagttctacacaagcctatcaatatttacatgcacaattaaaaaaactttgtggtaatgctgattgcaagtgtatggatggtgagtccaaagaaacaaccggacagcctgataactcccacgattcccatatgcctgcatcattagatgatgaacccgaagaagtgaatggaaagtgtaattgtaaagtgaaacatcgtccacaacctccgctagcacttccaccaccagcaccatcgggacctccagctgaagaccaaattgagcatgacaatagaggacgatcggaacgtggtgaccaaggcccactaccagcgcgacctcctcctcccccacaagctgcacaaccaccacaaccaaaaccaaaacgcactggagaaggcctcggtcgtaatctaccaccagctgacagaaataccaatctctccgattccgaagaagaagacgacgaagatgacgacgaagtccaggaggaggaggaaacgccaccgtcggaggcggaggaaggtgaaggacacgtcgagacagaggaggagacgaagccggtgaaggaaaagacggaaggggcgggggccacagaagtcacaaaacaggggtcggcaccaacggcaacaacaccaacagtagaagatatttgcgccacagtggccaaagcacttaagggcgacaaaagtctcaatgccgcatgtgccctcaaatatggcaaaaacaactcacgtttaggttggaaatgtataccaactagtggtgacaaaacagacacaagtgagaatggtgccccacgtcgtgctcgtagtgcccatggtggtaaaagtgatagtgaaaaaggttccatatgtgtgccgccgcgaagacgacgattatatataaaaaagatagtagattgggcggaatcacagtcgaagacagtaacaagtgttaatggagatggtaatgggtcacaggaagtagttagtgttaatggagctagtgagagtggtggtagtggtagtggtactgagtcacaggcgagtgatgtgtcacaaggtaacggcgcgtcgacatcgccacaagtggctctcctccacgcctttgtgaagtccgctgcaatagagacgttttttgcttggcataaatataaagtggataaagaaatagaggaaaaggaaaaacaggcagcacaaaatcatctagttcaacgtaaaacaagcgagaacccccaaaagaaattagaaggtggtgaaatacctgaagattttaagcgtcaaatgttctatactttaggagattatagggatattttagtgggggacaagactatgattgaggcgttagaaaagagtggtgacacgaaaatagaagatatatcggaaaaaataccaaaaattttagatggtgagaacaacaaagctgctggtggtggccccaaacaaccaaatagtggtaaaacaccacaagaatggtggaaagaaaacgcaaaacacatttggcatggaatgatatgtgctttaacatacaacacagacagtaatggaaaggacaaaaaaatacaacaggttaaagctacggacaacacagatcttttccaaaaactgaaaaaagacaacgactacgaaactgtgtcatttggtgctagtggtaccggcgccaaaagcaacgacgataccaaattaaaaaattttgtggtacgccccacatattttcgttggttagaggaatggggagaagagttttgtcgaaaacaaaaacataagttatatataattaaaaaagattgtcgtgataataagttttgtagtggtgatggcttgcgttgtgacgaaaaagttccagataagaaagatatttttaagcatttcgattgtcccagttgtgccagacattgtagatcttatagaaaatggatagaaagaaaaaaaacagaatatgagaaacaagaaagcgcatatagtaaacaaaaaagtaattacgtaaatggaagtaatggtgatggaggtaataataatgataaagaattttacacaaaactagaaacgtgcactaaagcaacaaactttttagaatcattaaaaggacaatgtattggtaataataatggaggcactgacataaaatttagtaatacaaatataacatttggatctgcagaagattgtaaaccttgttctgaatttaaagtaaattgtgaaaatggtagttgtgggagtgctaagcaaaaggattgcccaaataatacgattacttcacaaaatattaaaggtcttactgaccaagtagatatgcgtgttagtgataacactgaaagtggatttgaaggtgatttaggcatttgtcagggtgcaggtatatttaaaggtattagaaaagatgaatggaaatgtggtgatttctgtggtatagatatatgtactctggaaaaaaccaataatgggaaagaaagcgataaaaaatatatcataatgaaagaattcgttaaaagatggctagaatatttttttgaagattataatagaattcaaaaaaaattaaagacatgtaaagaaaatggtaaaggatccacatgtataagaagttgtgtagatgaatggataaagctgaaaaaggatgaatggcaaaaaattaacagtaattaccttgaccaaaatacaaaagaaaatcctgaaggtaataatttaagctcttttttggaggatggaccgtttaagaatgaggttgataaagctataaaaccttgtggtaatttaactgatttcaagaagtcaaagaaatgtaatggcacttccagatcaggaaatagtgaagagagcacaaaatatgatggtgttatatgtttgcttgataatcttaaaaacataataaaaacttgtcaaaacgtacctagtggcaaaccagatacaccgtgtcaaaaatcccccgcccccgttggagacgatgatgatccccttgaagaggaaaacccagtaacacaaccgaacatttgtccgcaaacatcagtggaagaaaaaaaaaaagaggaagaagaaaagtgtgatgaaaaggaggaagaagaagaaaaagaggaggaaaaagataaaggagatgaggaagtaaaagaagaagaaaaagataaaggagatgaggaagaagaagcagaagaagaagaagaagaagaagaggaaacagatagtcacatttatgaagactactctgattcagacgcagaggaagatgatgaagatgaagctgttacagaatccttatcaccttcagagtcacaaccaaaacgattgctacgagaatttccatcccccgaattaaaaaatgccatgttattttctaccatcctctggatggtaggtatcggttttgcggcgttcacttatttttttctaaagaaaaaaccgaaatcacctgttgacctcttacgtgtacttgacatccataaaggcgattatggaacacctaccccgaaatcaaaaaatagatatatcccctatgtgagtgatacatataaagggaaaacatacttatatgttgaaggagatacagacgaagagaaatatatgtttctgtctgatactactgatataacctcttccgaaagtgagtatgaagaattggatattaatgatatatatgtaccaggtagtcctaaatataaaacattgatagaagtagtattggaaccatcaaaaagtgatggtaacacaccaggtaagggtgatggtaacacactaggtgatgatatggtacctaccacgaatacatttacagatgaggaatggaatgaactgaaacaggattttgtatcacaatatatacaaagtagattaccaatggatgtaccacaatatgatgtatcaacggagagtccaatgaatataggaggtaatgttttagatgatggtatggatgaaaaaccttttattacttctattcatgatagggatttaaatagtggagaagaaattagttataatattcatatgagtactaacactaataatgatattccaaaatatgtatcaaataatgtatattctggtatagatttaattaatgatacattaagtgataacaaacatattgatatatatgatgaagtgctaaaaagaaaagaaaatgaattatttggaacaaattataagaaaaatacatcaaacaatagtgtagcaaaaaatactaatagtgatccaattatgaaccaattagatttgttacataaatggttagatagacatagagatatatgtgaaaattgggggaaaaaagaagatattttgaataaattgaatgaacaatggaataaagataatgatggtggtgatataccaaatgataacaaaaagttgaatacggatgtttcgatacaaatagatatggatgaaactaaaggaaagaaggaatttagtaatatggatactatcttggatgatatggaagatgatatatattatgatgtaaatgatgaaaacccatctgtagatgatatacctatggatcataataaagtagatgtaccaaagaaagtacatgttgaaatgaaaatccttaataatacatctaatggatcgttggaacaacaatttcctatatcggatgtatggaatatataa

SEQ ID NO: 2 - protein, 3553 aa, Plasmodium falciparum
MGNASSSEGEAKTPSLTESHNSARNILEGYAESIKEQASKDAKIHGHHLKGDLAKAVFRHPFSAYRPNYGNPCELDYRFHTNVWHRNAEDRNPCLFSRAKRFSNEGEAECNGGIITGNKGECGACAPYRRRHICDYNLHHINENNIRNTHDLLGNLLVMARSEGESIVKSHEYTGYGIYKSGICTSLARSFADIGDIIRGKDLYRRDSRTDKLEENLRKIFANIYKELKNGKKWAEAKEYYQDDGTGNYYKLREAWWALNRKDVWKALTCSAPRDAQYFIKSSVRDQTFSNDYCGHGEHEVLTNLDYVPQFLRWFEEWAEEFCRIKKIKLGKVKEACRDDSKKLYCSHNGYDCTKTIRNKDILSDNPKCTGCSVKCKVYELWLRNQRNEFEKQKKKYYKEIQTYTSKDAKTDSNINNEYYKEFYDKLKNEGYETLNKFIKLLNEGRYCKEKISGERNIDFTMTGDKDAFYRSDYCQICPECGVQCSGTTCTPKKVIHPNCKDKETYEPGDAKTTDITVLYSGDEEGDIAQKLQDFCNDKNKENDENYEKWQCYYKSSEINKCQMTPSSHKVPKHGYIMSFYAFFDLWVKNLLIDSINWKNDLTNCINNTNVTDCKNDCNTNCKCFENWAKTKENEWKKVKTIYKNENGNTNNYYKKLNNHFQGYFFHVMKELNKEEKWYKLMEDLKEKIDSSNLKNGTKDSEGAIKVLFDHLKDIAERCIDNNSKDSCPPSVDTKTNPCAKPPGSKPTKSVKQLAEHMQQKAQKLLGTRGGESKLKGDATRGTYNLGGQGNTLNGDICKITKNHTNDSRPNGEPCTGKDKVKNGFRLKIGTPWTNIVQKKKKKSYKDFYLPPRRQHMCTSNLENLSTSSKGLSNGSFASHSLLGDVLLAAKFEAQKIILVYKNKNNINIRKRITDPNDQATVCRAIRYSFADLGDIIRGKDMWNINSDAKDLQDRLEKIFKTINEKLPNEIQKRYTNRENKHLDLRSDWWEANRHQVWRAMKCATKGISNNNCNGIPIEDYIPQRLRWMTEWAEWYCKKQSQEYEKLEEKCGMCTGKGQGDGKDCTQKDKECSPCKKACDAYKKEIEKWEKQWKTVSAIYQILYAKARIVASNGGPGYYNTEVQKKDRSVYDFLYELHLQNGGKKGPPPATHPYKSVNTRDKRDATDDTTPTVYSTAAGYVHQEAHIGDCKEQHVFCDNNGNKEKYAFKNPPNVYVEACKCMTREAPPPPTTPSTPNPCAETGGVHTIKTVTDVAKILQGEANETMLKNSSNGNDKDESKLKGKAEEGDYSRGGTPSDFNNNLCGITQKHSNAHNDSQQPCYGKDQKRFNVGTEWSFKDNHRKRTHPEAYMPPRREHICTSNLEYLIHKRKKPIIEGDPNKIIHSLLGDVLLAAKYEAENIKKLYEENNNRKDQEGICRAMKYSFADIGDIIRGKDMWIENNDAKRLQTNLKEIFTKIKEKTGGTTYNEDNDPYLKLRADWWEANRAKVWKAMKCKTNGVDITCDSDHTPLDDYIPQRLRWMTEWAEWYCKAQSQEYKKLEEKCSQCKSKGKGGNECYRETKECNDCKQACEEYKRKIKTWADQWKVISNKYEDLYKKAQNPTNAVLKDNKDEKDKNVIDFLTQLQKANNGEKTGVHTVYSTAAGYIHQEARTRECQEQREFCDKKNGIDNTSYAFKDPPHGYATACDCINRSQTEEPKKKEENVESACKIVEEVLSKPRDKTTGGIDHCNPKYYPRKENYPGWNCTPGQFKSGHAGACMPPRRIKLCVINLQYLNEKKSPEELRKAFIQCAAIETYWLWQKYKKDKNGGVAQAKLNSGTIPDDFKRQMFYTFGDYRDLCLDTDISSKADTSTGVGKVKINIDSVFQKIDITNVEQRKPWWGKNAEAIWDGMLCALSYNTTNKNMDYNAHTKLNPTYGYNAIKSELEDFVNRPQFLRWFTEWSDEFCTERSIKIKELETKCNDCTVSESGTSDATGNKTCDDKDKCDECKRACTTYKTWLKNWKTQYKTQSKKYFDDKRKELYKSIDDVASSTQAYQYLHAQLKKLCGNADCKCMDGESKETTGQPDNSHDSHMPASLDDEPEEVNGKCNCKVKHRPQPPLALPPPAPSGPPAEDQIEHDNRGRSERGDQGPLPARPPPPPQAAQPPQPKPKRTGEGLGRNLPPADRNTNLSDSEEEDDEDDDEVQEEEETPPSEAEEGEGHVETEEETKPVKEKTEGAGATEVTKQGSAPTATTPTVEDICATVAKALKGDKSLNAACALKYGKNNSRLGWKCIPTSGDKTDTSENGAPRRARSAHGGKSDSEKGSICVPPRRRRLYIKKIVDWAESQSKTVTSVNGDGNGSQEVVSVNGASESGGSGSGTESQASDVSQGNGASTSPQVALLHAFVKSAAIETFFAWHKYKVDKEIEEKEKQAAQNHLVQRKTSENPQKKLEGGEIPEDFKRQMFYTLGDYRDILVGDKTMIEALEKSGDTKIEDISEKIPKILDGENNKAAGGGPKQPNSGKTPQEWWKENAKHIWHGMICALTYNTDSNGKDKKIQQVKATDNTDLFQKLKKDNDYETVSFGASGTGAKSNDDTKLKNFVVRPTYFRWLEEWGEEFCRKQKHKLYIIKKDCRDNKFCSGDGLRCDEKVPDKKDIFKHFDCPSCARHCRSYRKWIERKKTEYEKQESAYSKQKSNYVNGSNGDGGNNNDKEFYTKLETCTKATNFLESLKGQCIGNNNGGTDIKFSNTNITFGSAEDCKPCSEFKVNCENGSCGSAKQKDCPNNTITSQNIKGLTDQVDMRVSDNTESGFEGDLGICQGAGIFKGIRKDEWKCGDFCGIDICTLEKTNNGKESDKKYIIMKEFVKRWLEYFFEDYNRIQKKLKTCKENGKGSTCIRSCVDEWIKLKKDEWQKINSNYLDQNTKENPEGNNLSSFLEDGPFKNEVDKAIKPCGNLTDFKKSKKCNGTSRSGNSEESTKYDGVICLLDNLKNIIKTCQNVPSGKPDTPCQKSPAPVGDDDDPLEEENPVTQPNICPQTSVEEKKKEEEEKCDEKEEEEEKEEEKDKGDEEVKEEEKDKGDEEEEAEEEEEEEEETDSHIYEDYSDSDAEEDDEDEAVTESLSPSESQPKRLLREFPSPELKNAMLFSTILWMVGIGFAAFTYFFLKKKPKSPVDLLRVLDIHKGDYGTPTPKSKNRYIPYVSDTYKGKTYLYVEGDTDEEKYMFLSDTTDITSSESEYEELDINDIYVPGSPKYKTLIEVVLEPSKSDGNTPGKGDGNTLGDDMVPTTNTFTDEEWNELKQDFVSQYIQSRLPMDVPQYDVSTESPMNIGGNVLDDGMDEKPFITSIHDRDLNSGEEISYNIHMSTNTNNDIPKYVSNNVYSGIDLINDTLSDNKHIDIYDEVLKRKENELFGTNYKKNTSNNSVAKNTNSDPIMNQLDLLHKWLDRHRDICENWGKKEDILNKLNEQWNKDNDGGDIPNDNKKLNTDVSIQIDMDETKGKKEFSNMDTILDDMEDDIYYDVNDENPSVDDIPMDHNKVDVPKKVHVEMKILNNTSNGSLEQQFPISDVWNI

SEQ ID NO: 3 - DNA, 8985 bp, Plasmodium falciparum
atggggtcacaaacatcaaaattttctaaaactgttgttggaaatgaaacacacaacagtgcccgaaatgttttggaaggttttgcaaaagatataaaaagggatgtatcaaataacgcaaaaagacatggaaaagttttgaagggaaatttgagagatgccaaattttatcatgattattctaagttaagagacatacctagaagtccctgtgatcttgatttttggtttcatacgaatgtttggagggacaaagcatatgaaagagatccttgttatggcagacaagcaaaaaataattacaatttggaaggagcagtatgtacgaatagtaaaataaaaggtaatgaaaacaagataaatgacattggagcgtgtgccccatatagaagacgaaatatatgcgattataatttagaacatctaaatgaaagaaatgttttaaatactcatgatttattgggaaatgtgttagttatggcaaaacgtgaaggtgaatctattgttgagaaacatccaaatagaggatcttcagaagtatgtattgcccttgcaagaagttttgcagatataggagatattttaagaggaaaagacatgtatgtcgggtatgatgaaaaagaaaaaaatcgaagaaaacaattagaaaataagttgaaagatattttcgataatatatataaggatttgacgaagaagaaggggaggaatgggaagaagtcggcgctacaagaacgctacaatgatcctaaaggagatttttttcaattacgagaagattggtgggcgcttaatagagaagacgtatggaaagcattaacatgttctgcggatgacagtgaagattatttcatacaatcagaaggtgttacaaaatcatttacaaatcctaaatgtggccatggtgacaatgaggttcttacaaatcttgattatgtccctcaatttttacgttggttcaccgaatgggcagaagagttttgtaggataagaaaaattaaattaggaaaggttaaaaacgaatgtcgtggtgagacatcaggtaaaaggtattgtagtggcgatggttatgattgtactaaaacagatatatcacgtaatattttttatatggatttagattgcccacgttgtgaagaagaatgtagaaaatatgatgaatggattgaaaataaagaaaatgaattagataaacaaaaaaataaatacactaaagaaattgaaaaattaaaagataattctaagagcaattatgataaaaatttttatttaacacttactaaaaaatatggttcaattaacttatttttagatacattaaaagaaggatcacattgtagctacaataccatagaagataaaatagattttaacaaagcaaatcaaacatttactagttcgaaattttgtggggcatgtcctttttatggagttaaatgcaattggaaaacatgtacagaggttaaggaaaatgagtacaaaaaaaaaaataaggttgatagtacacatacgacagaacaacctactgctattgatgtactagttactcatattagaggaacaaatattcccgaagatttaaaggattgtaaaaaatatggtctttttaaaggtatgcgaaaacaagcgtggaaatgtcaatatataaatccatatgatgaatgcaagttgagtccttttgtgaaagatatagatgttgatgatcgtattttatttaaagtattgtttgagcgttggttaaaatatttcatacaagattttaataatgttaaggacaaaattaatagatgtacaaaattcgaaaagggaaaagataatacatgtattaaaggttgtaaacataaatgcgaatgtgtggaaaaatggataaaaataaaggaagcagaatggaaaaaaataaatcaacattataaccaacaaaaaaaacattatacctatagtgttcctcgttgggttaatagttatttgacgcaccagcacttctctagtgattttattaatgcattagaagcttttaaaaatatacggggattagaaaatttgaaggaatgtagtagtgacacttgtaaaattgaaaaaattagaactatagatgatgatttaataaaagaattaatttctaaacttaaagataaatgtgctatgtgtaaaaaccaacataaagcaaccaaaggtaaagaatgttgtggtaaattacctaaaactctaaatgatcaagatgatgaagaagacgaagaatacgaagcaccaccaccaccaacacctcctcgcacccaaaaaaacccctgcgttaatggtcaaaaccagaaggttcgtaaaatcaggagcgtgagacgagtgccgaaaaggatgcaaaaacaagcaagtgttcgtgttcctcgtgctcggcagggaggggagagggagcaggttgtaaagaacgggcgccaggaccaccacctccagcgggtgcttttggtgggggaggcggaggcggaggagccggagacggcggaggaaaagaaggaggaggaaaaggaggaagacacggacgggaaggtacagccaccaccagcagcaacaacacccggggtgaagccgccatgtgacatagttgaaaaacactttaaagataagcacgataatactggtgcaatagatcattgtaatccaaaaaaggattatcctccatggaaaaacgacaaaagtttagtggatgaagatggtgtgtatatgcctcctagaagacaaaaattatgtgtaattaatttagaacattttaaagagaatacatcagatgatttgagagaagcattcattaaatgcgctgccgcagaaacttatttgttatggcaaaaatataaagaggataacaatggtggtgaagatctacaaaaccaattaaaaagtggaaaaattcctgaagattttaaacgtcaaatgttctacacatttggagattatagagatttcttatttggaacagatatatcaaaattaaataaacatacagaagctgttaaaactaatatagatagaattttcccaccaactgagcgaacaaatgatacaatacgtaaagaattttgggaaaaaaacgcagaatctatttggcaaggaatgttatgtgctttaagttataatagtaatgacaaaaaaatggatccagatgtacaaaaagaactaaactccacctacaactacgataccataaaaaataatctagaagactttgcgaacagacctcaattcttacgatggtttattgaatggagtgatgaattttgtcgtgaacggaagaagaaggaagaaaaggtaggatcagcttgtaagaatgattatgagggatgtgcaaacactaaggataatggtaatggtaattgtgttaacgcatgtaatgcatataaaaaatacattacagacaaaaaagaacaatatgaaaaacaagcaaagaaatttgatattgataaaagtcagaataaaccaggatatgaagattattctggcaaaaaggcttctgaatatttgaaagaaaaatgtataaactcatcatgtgattatatgctaaaattaaaagataattccaattactgggaaaaacctcatacaacatacgacgataattcacttcaaaacaaatgtagctgccccctttctccctgcgaaatcgtggataaaacactgggcgacaaaacctcaaagtcttacgccgaagggtgcaaatggaaatatgggaagatgccactaggtttgggatggttatgtaatgacaaagagggtgagaagggtaaggaggacggtttatgtataccgccaaggagaaagagattatatgtaaaagatttagagacattcagcgatcacacaacagtaggtctccgagaagcttttattaaatgtgctgctgtagaaactttttttgcttggcatgaatttacaaaggaaaaagaaagagagtataaagaagaaaaacaaagaaatggagaacttggatttatcgacgaaaacgatcaaataccaaaggacccagataatccacagaacaaaataagaaaaaatggagaaatacatgaagagtttaaaagtcaaatgttctatacccttgcagattatagagatatattatttgggaataatataggtattggtaacgacatggggaaagttaaaagtaatatagataaggttttcgcaaatagtagtggcaaaacacctactgccaaaaaaacaacaccaaaagaatggtgggaaaaaaatgcaaaagatatatgggaaggaatgttatgtgctttaagttatgatacgaaaacaaaaattaagaatgaagaactgcgcaagaaacttatagatccaaagaacagcaactacatgtacgaaaaagttacatttagtagtgataataacacaaatttgtcaaaatttacagaaagacccccgttttttcgatggtttcaggaatggggagaagagttttgtcgaaaaaaaaaaataaaaattgataaaattgaaaaagaatgtcgtggaccatatggtcgaaatcattgtgatggcgatggattcgactgtagtgaaataggcccaaatgagaatggaagttttgcgatttttaaatgtccaagttgtgcaatttcttgtcgatcttataaaacgtggataaacacaaaaaaagacgaatttaaaaaacaagaaaaactatataataaagaaattaaggataataaaagtaattatgataatatatatgataaagaatttgttaaaaacctttgtacagattataagtctgttgactcatttttaaaaaagttaaaagaaggcccatgttgtaacaaaaatactaaagacagtaaaatagattttaaggatacagaggagacatttagaaacgctgaatattgcgatccatgtcctgtatttggagttatttgtaataacggtgattgcagtaattctacagaaaagaagtgtgatgcacaagaatttaaggttacatatgatgtaaaaaataaggaaaaccctaataaagaagtaaatatgcttgtcagtgataaaacagcaaagaaatacccaggtgatttaaacggtgtttgcgaaaattcaagtatctttgaaggtattagagaagataaatggtcatgtggttatttctgtggtttagatatatgtacacccaacaaaactacaggtgatatacatgataaacaaaatgcaccaattagagtactgtttaaacgatggatagaaaattttttaaaagatcataacaaaattaaagacaaaatttctttatgtataaataatgaaaacagaaatatatgtacagatgtttgcagaaaaaattgtgaatgtatagataaatggatagagatgaaaatgaaagaatggaaaatagtacgcgatcgttacgtcaaacaatataatgttgctgattcagtagtttacgaagtgagaagatttttagagggcttgcaacctcaaaatgaccttgaaaaagttaaaggagatgttaatgatttacgtgatttagaggaactaagtgaatgtactaatactgtatcaacagaaaatagaaaatgtagaaaaaaggatgtagtagaaagtttgcttaataaacttaaaaatgaaatacgtcattgtaaaaatgaacgtgacgatagtatgggcaaggaaagttgcaaaacattacccgaacctacagacgatccacaaacagatagtgatacccacgacacacctgacataccaccaggtgacgttgcacccactttttgtaatgttccagcaaatccatgtggcgacaaaagcgccaccaatgtggtaaatgtgacagaggtggcgaaggaaatgcacgaagaggcacacaaggatatgttagagaggagtgttaaaaaggttgagagtaaggttaaggatagtacggttgagagtgtgttaagggctgatgcatcaaaaggtgaatataaacatgaaggtaatccagatgacttgaaacacaacatgtgcaatataacgaaggaacataccaattatcaaaaacgtggtggttataattatcgaggaccatgtacgggtaaaggtaatggtaaagacacaagatttgtcataggaaccatatggaaagatgaagacgaaaaagatgaaaccattaaagttctgttgcctccgcgacgtcgtcatatgtgtacatcaaatttagaatatttacttcatgttaataagggcccacttctaaaagttgaacctgataaaattaatcattcctttttgggggatgttttgcttgcagcaaaatatgaagcagaattcataaaaaccaattatacgagattaaatggccaaaatgacaatggagctaaatgtagagctatgaaatatagttttgctgatataggtgatattatacgaggaaaagatctgtggggaattcaggacttcaaggatctacaaactaagttagtaacaatatttggtaaaattaaagaggaaattcccgatattaaaaaaaaatatagtagtgaaaatcccccatatacgacattacgtgaacattggtgggaagcaaatcgagccaaagtatgggaagcaatgcaatgtccaacaataccaccagtcaccacaagttgtgatactaccactgttacccctcttgtggattacatcccacaaagattacgttggatgaccgaatgggccgaatggttctgcaaaatgcagtcacaggagtatgaggtgttagtgaaacagtgtaggaattgtaggagtggaatatgtgagaatggtaaggatgactgtgtcaagtgcacacaagcttgtaatacatataaacaaaaaataaaaaaatgggaagatcaatggaaggaaatatcaaaaaaatacaaaacattataccaacaagcaaaaggcagtgttaatggtgctactactagtagtactacagatgagaaagacaaagatgtcgttgatttcttgaaaatgttacaccaaaaaaatactgataataccatatatactactgctgcaggatttatacatcaagaagcacatatgactgattgtcaaaaacaaacaattttttgtaaaaacactagttataacgacaagaagaaatatgcttttcgtcatccaccacatgatcatgatgatgcgtgtgcttgcaggccaccatcaacgccagtagacgtctcccgcaaactagacacccaacgtgaccccaaaaaagaggaatctgaacctgaatccgaagaagaagaagacgatgccgaagaggaggaggagccggcaaaggagacggctaccacagagacaacacaaccagcagcaccagcgggaccaccggtaacaccagtaccagaactaccgggaccacccgcaccagcgggaccagcagctgatggccccattgaggatgacgaagacgccgaaaacgaagacgatgatgacgtcggctccgccaccggcacagaagacgatgacgacgacgaagatgacgacgacgaagacgaagaggactcagcagacgaaggtgaaggtgaaggcgacggcggtgacgtcggcgaggaggaagatgaagatcacggcggccaggaggcggagggggtggtaccacaaccagcagcaccacaaccaccaaccccacaactcttggatgaccccctccttaaaaccgccctcatgtcttctaccatcctctggatggtaggtatcggttttgcggcgttgacttactttttactcaagaaaaaatccaaatcttctgttgacttgttgcgtgtactgaatatcccgaaaggagattatgaaatgcctacgttgaaatccaaaaataggtacataccatatagaagtggttcatataaaggcaaaacatatatatatatggaaggagatagtgatagtggacactactacgaagatacaactgatattacttcatccgaaagtgaatatgaagaattggatattaatgagatatatccgtatcagtcaccaaaatacaaaacattgattgaagtggtactagaaccatccaaaagtaatggtaacacaccaagtaagggtgatggtaacacactaggtgatgatatggtacctaccacgaatacatttacagatgaggaatggagtgaattgaaacatgattttatatcacaatatatacaaagtgaaccactggatgtaccaaaagttggtgtatcaaaggaattaccaatgaatataggaggtaatgttttagatgatggtataaacgaaaaaccttttattacttctattcatgatagggatttatatactggggaagaaattaaatataatattaatatgggtactaatagtatggacgatccaacatatgtatcaaataatgtatattctggtatcgatttaattaatgacacattaagtggtaatcaacatattgatatatatgatgaagtgctaaaaagaaaagaaaatgaattatttggaacaaattataagaaaaatacatcaaataacaatgtagctaaattaacaaatagtgatcctattatgaaccaattagatttgttacatacatggttagatagacatagagatatgtgtgagaagtggaataaaaaggaagaattgttagataaattaaatgaacaatggaataaagataatgatggtggtgatataccaaatgataacaaaaagttgaatacggatgtttctatacaaatagatatagatgaaaataaaggaaagaaagaatttagtaatatggatacaaacgtggatacacctactatggatagtatattggatgatttggaaacatataatgaacctttttatgatatatttgaggatgatgtgtattatgatgtatatgatgaaaacccatttgtggatgatatacctatggatcataataaagtagatgtacctaagaaagtacatattgaaatgaaaatccttaataatacatccaatggatcgttggaacaacaatttcctatatcggatgtatggaatatataa

SEQ ID NO: 4 - protein, 2994 aa, Plasmodium falciparum
MGSQTSKFSKTVVGNETHNSARNVLEGFAKDIKRDVSNNAKRHGKVLKGNLRDAKFYHDYSKLRDIPRSPCDLDFWFHTNVWRDKAYERDPCYGRQAKNNYNLEGAVCTNSKIKGNENKINDIGACAPYRRRNICDYNLEHLNERNVLNTHDLLGNVLVMAKREGESIVEKHPNRGSSEVCIALARSFADIGDILRGKDMYVGYDEKEKNRRKQLENKLKDIFDNIYKDLTKKKGRNGKKSALQERYNDPKGDFFQLREDWWALNREDVWKALTCSADDSEDYFIQSEGVTKSFTNPKCGHGDNEVLTNLDYVPQFLRWFTEWAEEFCRIRKIKLGKVKNECRGETSGKRYCSGDGYDCTKTDISRNIFYMDLDCPRCEEECRKYDEWIENKENELDKQKNKYTKEIEKLKDNSKSNYDKNFYLTLTKKYGSINLFLDTLKEGSHCSYNTIEDKIDFNKANQTFTSSKFCGACPFYGVKCNWKTCTEVKENEYKKKNKVDSTHTTEQPTAIDVLVTHIRGTNIPEDLKDCKKYGLFKGMRKQAWKCQYINPYDECKLSPFVKDIDVDDRILFKVLFERWLKYFIQDFNNVKDKINRCTKFEKGKDNTCIKGCKHKCECVEKWIKIKEAEWKKINQHYNQQKKHYTYSVPRWVNSYLTHQHFSSDFINALEAFKNIRGLENLKECSSDTCKIEKIRTIDDDLIKELISKLKDKCAMCKNQHKATKGKECCGKLPKTLNDQDDEEDEEYEAPPPPTPPRTQKNPCVNGQNQKVRKIRSVRRVPKRMQKQASVRVPRARQGGEREQVVKNGRQDHHLQRVLLVGEAEAEEPETAEEKKEEEKEEDTDGKVQPPPAATTPGVKPPCDIVEKHFKDKHDNTGAIDHCNPKKDYPPWKNDKSLVDEDGVYMPPRRQKLCVINLEHFKENTSDDLREAFIKCAAAETYLLWQKYKEDNNGGEDLQNQLKSGKIPEDFKRQMFYTFGDYRDFLFGTDISKLNKHTEAVKTNIDRIFPPTERTNDTIRKEFWEKNAESIWQGMLCALSYNSNDKKMDPDVQKELNSTYNYDTIKNNLEDFANRPQFLRWFIEWSDEFCRERKKKEEKVGSACKNDYEGCANTKDNGNGNCVNACNAYKKYITDKKEQYEKQAKKFDIDKSQNKPGYEDYSGKKASEYLKEKCINSSCDYMLKLKDNSNYWEKPHTTYDDNSLQNKCSCPLSPCEIVDKTLGDKTSKSYAEGCKWKYGKMPLGLGWLCNDKEGEKGKEDGLCIPPRRKRLYVKDLETFSDHTTVGLREAFIKCAAVETFFAWHEFTKEKEREYKEEKQRNGELGFIDENDQIPKDPDNPQNKIRKNGEIHEEFKSQMFYTLADYRDILFGNNIGIGNDMGKVKSNIDKVFANSSGKTPTAKKTTPKEWWEKNAKDIWEGMLCALSYDTKTKIKNEELRKKLIDPKNSNYMYEKVTFSSDNNTNLSKFTERPPFFRWFQEWGEEFCRKKKIKIDKIEKECRGPYGRNHCDGDGFDCSEIGPNENGSFAIFKCPSCAISCRSYKTWINTKKDEFKKQEKLYNKEIKDNKSNYDNIYDKEFVKNLCTDYKSVDSFLKKLKEGPCCNKNTKDSKIDFKDTEETFRNAEYCDPCPVFGVICNNGDCSNSTEKKCDAQEFKVTYDVKNKENPNKEVNMLVSDKTAKKYPGDLNGVCENSSIFEGIREDKWSCGYFCGLDICTPNKTTGDIHDKQNAPIRVLFKRWIENFLKDHNKIKDKISLCINNENRNICTDVCRKNCECIDKWIEMKMKEWKIVRDRYVKQYNVADSVVYEVRRFLEGLQPQNDLEKVKGDVNDLRDLEELSECTNTVSTENRKCRKKDVVESLLNKLKNEIRHCKNERDDSMGKESCKTLPEPTDDPQTDSDTHDTPDIPPGDVAPTFCNVPANPCGDKSATNVVNVTEVAKEMHEEAHKDMLERSVKKVESKVKDSTVESVLRADASKGEYKHEGNPDDLKHNMCNITKEHTNYQKRGGYNYRGPCTGKGNGKDTRFVIGTIWKDEDEKDETIKVLLPPRRRHMCTSNLEYLLHVNKGPLLKVEPDKINHSFLGDVLLAAKYEAEFIKTNYTRLNGQNDNGAKCRAMKYSFADIGDIIRGKDLWGIQDFKDLQTKLVTIFGKIKEEIPDIKKKYSSENPPYTTLREHWWEANRAKVWEAMQCPTIPPVTTSCDTTTVTPLVDYIPQRLRWMTEWAEWFCKMQSQEYEVLVKQCRNCRSGICENGKDDCVKCTQACNTYKQKIKKWEDQWKEISKKYKTLYQQAKGSVNGATTSSTTDEKDKDVVDFLKMLHQKNTDNTIYTTAAGFIHQEAHMTDCQKQTIFCKNTSYNDKKKYAFRHPPHDHDDACACRPPSTPVDVSRKLDTQRDPKKEESEPESEEEEDDAEEEEEPAKETATTETTQPAAPAGPPVTPVPELPGPPAPAGPAADGPIEDDEDAENEDDDDVGSATGTEDDDDDEDDDDEDEEDSADEGEGEGDGGDVGEEEDEDHGGQEAEGVVPQPAAPQPPTPQLLDDPLLKTALMSSTILWMVGIGFAALTYFLLKKKSKSSVDLLRVLNIPKGDYEMPTLKSKNRYIPYRSGSYKGKTYIYMEGDSDSGHYYEDTTDITSSESEYEELDINEIYPYQSPKYKTLIEVVLEPSKSNGNTPSKGDGNTLGDDMVPTTNTFTDEEWSELKHDFISQYIQSEPLDVPKVGVSKELPMNIGGNVLDDGINEKPFITSIHDRDLYTGEEIKYNINMGTNSMDDPTYVSNNVYSGIDLINDTLSGNQHIDIYDEVLKRKENELFGTNYKKNTSNNNVAKLTNSDPIMNQLDLLHTWLDRHRDMCEKWNKKEELLDKLNEQWNKDNDGGDIPNDNKKLNTDVSIQIDIDENKGKKEFSNMDTNVDTPTMDSILDDLETYNEPFYDIFEDDVYYDVYDENPFVDDIPMDHNKVDVPKKVHIEMKILNNTSNGSLEQQFPISDVWNI

SEQ ID NO: 5 - DNA, 10041 bp, Plasmodium falciparum
atggggaatacacaatcatcagaggaagaggaagctaaaagccctagtttaacagaaagtcacaacagtgcaaggggtgttttggaagaaattggaaaaaagataaaagataagacagaaaaagagagtaaacatgtaaggcaattgaaaggaaaattatcaaatgcaaaatttgctgatcgattgtataaggaatctggtggtgatttaaggtctgcttattcagatgcttgttcacttacatacaaatttcatactaatataacaactgatgggggggatggaaggcatccttgtcatggtagggaaaacaatcgtttttctgaaagtcaagaatatggatgtagtaatgtatacataaaaggaaatgaaaataacagtaatggtacagcatgtgtaccaccaagaagaagacatatatgtgatcaaaatttagaatttttagataatcctcacactgatgatactgatgatttgttgggaaatgtgttagttacagcaaaatacgaaggtaattatattgttagtaatcatccagataaaaacagcaatggaaataaatcaggaatatgtacttctcttgcacgcagttttgcagatataggagatattgtaagaggaagagatatgtttaaatctaatgaaaaggtagaaatcggtctaaaaaaggttttcgagaaaataaataatggattgaagaaaataggaattaatgattataatgatatatctggaaattattataaattaagggaagcttggtggacggctaaccgcgatcaagtatggaaagccataacatgtagagccccaaacggggctaattattttagaaaaggtttagatggaaaaataattttttcagataatggaccatgtggtcgtaaggaactaatcgttcctacctatttagattatgtccctcaatttttaagatggttaaatgaatggtcggaagagttttgtcgaataaaaaatataaaaataggaaatattaagaaatcctgtactggagaaagtaataataaacattgtagtcgtgagggttacgattgtaataaaacaaatctaagacttaatgaaatttttatggatctagaatgtccacgttgtgcagatgattgtaaatcgtatgaaacatgggtagaaaaaaaaaaaaaagaatttaataaacaaaagaaaaaatacgaaaaagaagttgatgctacacaaaataatgataataacgaaaatggaatctataataaaaaattttatgatgaactaaaaagttcatataaagaagttaatagtttttttgaattattgaataaaggaccaatatgtgaacatattgataaaaaaattccaatggactataataatactgagaaaacattttcccgttcagagtattgtaaatcatgtcctataacagatattttatgtgatgataatgaatgtaaaaccattaatgaatttaaatgtagagaaataaaaagtatgcctaatataagaaaaaacgaaaatgaaacccctattgatattgatattctggttaatgttaataacaaaaaggttattacgcatgatttaaagaataattacgaaaactgtgatctttttaaaaaactaggagaacaaaaatggaaatgtaaatataaatgttacttagatgtatgtgaaccgagaaatttggatagtaatatatataatgaacgatatatctcaattaaagtactatttaaacggtggttagaatatttcttagaagattacaataaattaaaggaaaaattgaacccatgcatgtataatgtacaagaaatcgtatgtataaatgaatgtaagcaaaattgtgaatgcgtagaaaaatggataaaagaaaaaagggaagaatggaaaaaaataaaagatcgttacgtacaacaatatgaaagtaaagatgaagatgtttcttctaaacttaaaaaatttttgaaacaggaactgtttactaattatgttaaaaatgccttggacaaggatgaaacgttagatagtatgaaagaatctactgaatgcattgatcctaataaacccaaaggaaaaccatgtaacaataacgatgtcataaatattttacttaatagacttgaaaaacaaatcgataattgcaaaaagaagcacgaagaaaagggagaaaaaccttgtgttgatatacctaaacttctaaatgatgaagatgaggacgaagatgacgacgaaacaccacgcgcccataatccgtgtgtagataaaaatgattctcaacccactaaaactgtgagttatatcgctagacaaatgcatcgaagggcaaaagcacaaatgacaaaaaatagtgttgttgatggtgataataagttggaaggcgatatatttaaggttacatttagaaatggcggggtcggaaaaaacctgaatggagatatttgcaagattgacaaaacgtattccaatgacagtcgtggtactcctacagatggaccttgtgaaggcaaaggcgatcggtttaaaataggaacggactggcaaggtgatagtttcgtaaacccacaataccgtgggatttatatgcctcctagacgtcaacatttttgtacatcgaatttagaaaaattagatgttagtagagtcataagaaatggtaatgctagcaattcattattgggtgatgtgctgctcgcagcaaagtatgaagcagaacgaacaaagaaccattatgtatctaaaaaggaagaacattccgaagcttgtcgtgctgtgcgttacagttttgccgatttaggagatattatacgaggaaaagacatgtgggataaaaatcatggcgagaagaaaacacaagaaaatttagaaagaatatttgctaaaattaaagagcaacttcttaatagcagtatcaaagataaatataaggatgatgacaaagcaacacccaaatataaacaattaagagaagattggtgggaagccaatcgttcacaggtatgggaagcaatgcaatgcccaccaaaaaacggtactttcccatgtaaaagtgatcatacaccgctacatgactacatcccccaaagattaagatggatgaccgaatgggcagaatggtactgtaaagaacagtcacggctgtatggagagttggtggagacgtgtggtaagtgtatgcataagggaaaatgtaagcaaggtaatggccattgtgtaacgtgcaagccagcatgtgaaaaatataaaaaatttattaatacatggcaacctcaatggaaacaaatggaacaaaaatactcccagttatacgaagaagcaaaaaagtataatgatagtagcagaaaagataccacaaacaaagacgattatgtccttcaattcttgaacaaattactcacgcaaaacaaaggaaataaaacatatgatactgctgaaggatatgtacaccaagaagcacatattagtgattgtcagaaacaaacacaattttgtaaaaaaagaaatggtgagatcccaagtagtgatacagagaccgacaacaattatgcctttcgtcctcaaccacacgaccatgatgaggtgtgtgagtgtaatactagacagaagacgaaggtacggaaaaaaaaaaaaaaagttgatgcatgtgaaatggcaaaaacacttttgcacaacaacgatggaaccataagaataggacaatgcaaacgtaaagatgaaggaaatgcagaatatccaaaatgggattgtaattctcagattcatacaacacataatggagcatgtatgcctcctagaagacaaaaattatgcgtatatttttttgcaaatccatctcaaataggaagtataaataaacaagataatttaagaaaagcatttattatatctgcagcagcagaaacatttcgttcatggcagtattataagagtaagaatggtggtgaaaacctccaaactcaattaaaagatggaactattcctgacgattttaaacgtcaaatgttctatacatatggagattatagagattttttatttggaaccgatatatcaaaaggtcttggtgaagggactgccctagaaaagcaaataaatattcttttcccaaatggtgtccgaaaaattcctaatgaaaaaacacgtgaaaagtggtggacagatcacggacctgagatatggaaaggtatgttatgcgccctaacaaatggtctcagtgaaagcgaaaaaaaaacaaaaatattcgacgactactcacacgacaaagtcaaccaatccaaaaatggtaacccttccctcgaggatttcgcaaaaaaacctcaatttttcagatggtttattgaatggagtgatgaattttgtcgggaaaggaagaagaaggaagaggaggtggaaagggattgtaaggatgaatatgagggatgtgaaaaggagaaaaatggtaaatgtgttaccgcatgtaaagcatataaagaatacattacaaacaaaaaagaagaatatgatagtcaaaaagggaaatttgacgttgaaaaaacagagaagaaacaaggatatgaagattattctgagaaacaggcttctgaatatttgaaagaaaaatgtataaaatcatcatgtaattgtatgaagaaagttacagaaatttccaattactggaccaaccctcataaaacctacgacaccgaaaatcttggaatcaaatgtgaatgccccccttcaccctgcaccatcgtggatggcatcctcagcccacaaaattcgagttcgtacgccgaagggtgtaaatggaaatatgggaagatgtcacaagggggtacggaatgggattgtagtaaaaaaagtgggggtgaaggtggtaatgaggacggtgatgttgtatgtatacctccaaggagaaggagattatatgtaaagaatttacaggatttgactggtgaagaatcactagtggatttacgaaaagcttttattaagtgtgctgctatagaaacattttttgcttggcatgaatttaaaaaagaaaaagaaagagaggaaaaagaaaaaaatgaacaagatgtacaatataaatcatctgtcttagaaaatcttcaaaagcagttaaaaaatggagaaatagatgatgagtttaaaagacaaatgttctatacatttgcagattatagagatatatgtttagggaaggatataggtaacgacgtggatggaattaatgaaaaaatagatacaattttgcaaaaaaatggaaaacctaataatatcgaagaatataaaaaatggtggcaaaaacatggtcatgagatatgggaaggaatgttatgtgctctaagctacaataccgaaacaaaagagatggataaagaacttcgcaacaaattaaccgaacaaaagaacggtaacaaaaacacgtacgacaccgtcacaattagtggtggtcccattggtaataccaaattggagaaatttgcatctaggcccccattttttcgttggttagaagaatgggcagatgagttttgtagaaaacgaacacataaattggaaaaaattcaaaatgaatgtaagggagtaagtggtacaaatcagtgtgatgatgatggttttgactgcgatgaaatgtgtccaaaaaaggatgggagttttgaaacgtttaaatgtctgagttgtgccaaatcttgtagattttataaaaagtggataagtagaaaaaaagaggaatttgataaacaaagcaaaaaatacgaaaacgaaattgacgatgttaaacataattctgataacatatatggaaaagactttcttgaaactcttgatcaacaatataagtctgttgaattatttttagaaaaagtaaaaggaccatgttctattaataataataatgaagaatgtaaaatagattttaataaaccaaaggatacatttggtcatgcaaaaaattgtggtccatgttctgaaattagattcaagtgtatagaggataatagcaattgggttactacaaatacatgcaataaaacaacttttaagtttacagaagataataaagatacgaaagaagatagtgaacaattaggtatgcttattagtgataatacagtacaaaattttgcagatggtttacagaatgattgtaaagatgcagatatctttaaaggtcttagaaaagaccaatggtcatgtggttatttttgtaatttagatatatgtagtctgaaaacttctcatggggaaaacaattataaacaaaatatattaattagggcattgtttaaacgatggttagaacattttttagaagactataataaaattaatgacaaaatttcacattgtatgaaaaatggtgaaggatccacatgtataaaaggatgtgaaataaaatgtaattgtgtaagtaattggataaagaagaaaacgttagaatgggaaatagtacgagatcgtttctttaaacaatataatgttgattcagaaaaatcttttacagtgaaaagttttttagagcaggctccatttgacagtgatgttcaaaaagctataaaaccttttgaaaaattacgtgacttcgaggattcaattgtatgtaatggaactacgagcgcacgaaaggaaaaaggtacagaaaaggatgtcgtaatatgtttgcttgataaacttcaaaaacaaatagaaacttgtcaaacgaaacataaagaaacatctggaaatacatgttccccacccccaaaccccgacacacaaacagacacaccattaccacttgagtcttttcctccccctttttgtaacgttcctcctaatccatgtggcgacaaagatgcgaccaacgtggttggagttgaagtgctggcgaaggaaatgcaggaggcggcacataaaagcatgttaagtcgtagtgctgttgatagtggtaagggtgataagggtgagagtagtagtggtaagagtagtttggaaggagatatatccttagcagaatttaaaaatggctttaatccaagtgggttgaagaacgtatgtcaaataacggaaaaacattcctatgctaatggtgcatcaaaggatccttgtaatggaaaaggaaacggcaaggaccagagatttaaaatagaaacccaatggaaagatacaggcaaaagcggtaaacacgttgacgtctatttacctccacgacgcgaacatatatgtacctcaaatttggaatatttacttaagggtaatagtgatcagattatgaaggttggaaataacaaaattaatcattcctttttgggagaggtattgctagcagcaaaatatgaagcagaattcataaaaaccaattatacgagattaaatggccaaaatgacaatggagctaaatgtagagctatgaaatatagttttgctgatataggagatattgtacgaggaagagatctgtgggaacataatgattttaaaaagctagaacgagatttggtaaaaatatttggtaaaattaaagagggaattactgatgagacaaccaaaaaacaatatgaaaaggatgacacagacaataaacaattacgttgtgattggtgggaagctaaccgtgatcaagtatgggaagcaatgcagtgtaaaacaacaataccaccagtcaccacaagttgtgatactaccactgttacccctcttgtggattacatcccccaaagattacgttggatgatggaatgggcagaatggtattgtaaatatcaatcgaaggcatatagtgagttgaggaaggggtgtgaggattgtaggagttggaaatgtatgaagggtgatagtaaatgtgagaattgcacaaaagcttgtaaagactataatagtaaaatagaaccatggaaacagcaatggacaaaaataaaagaaaaatacgaagaattatacaaaaaagcacaaaatagtgatacctctaatagtggtacaacatatcccaaagatgagaaagatgtcgtttcttttttgtcaaaattacacgaaaaaaataaagacaataagatatattatactgctgcaggatatatacatcaacaagcaaaatatttagattgtacacaacaaacacatttttgtgataaaaaaaatggcgagacattacctagcggtagagacaatgacaaatatgcttttaagaaaccgccaaaaaaatatgaacgagcatgtaaatgtcacgagaaacaggaaccaccacctcctaaggtaccagaagattcagaggacgatagagaacgatcagaacctggtgaagatgcactccctgtactcccaccagaagaaatagaacaagaggaagaacccgaagaaacttccgtagacactacacaagatgaggaggaaccagcatccgaaggaggtggcccatcgggatcaccaacagaagaaagtggggaaccaagagaaaatagtgatagctccgaccccaaacctgaccaaaaccccgaagccaaccccgaacaaacaccgatactcaaacccgaagaagaagcaccaccaaaatcaaaaccacccgatggagatcgtggcgtaggacgttctttaggaccaaccccacgttctgaagttgaacccgaggaatccgaaaacgaagacgtcgaagacgaagatgacgaagaggaggaagaagaccccgacgacgaccccgaggcggagtcggaggaggaagatgaagatcacggcggccaggaggcggaggcggtaccaccacaaccccaagcaccagcacctctaccccctcctccaccacctttacccccccttaaaaccgccctcatgtcttctaccatcatgtggagtgtaggtatcggttttgcggccatcagttactttttactaaagaaaaaaccgaaatcacctgttgacctcatacgtgtaattgatatccataaaggcgattatggaatacctacattggaatccaaaaatagatatatcccctatgtgagtgatacatacaaaggcaaaacatatatatatatggaaggagatactagtggagatgaaaaatatggatttatgtctgatactactgatataacttcctccgaaagtgagtatgaagaattggatattaatgatatatatgtaccaggtagtcctaaatataaaacattgatagaagtagtattggaaccatcaaaaagtaatggtaacacactaggtgatgatatggtacctaccacgaatacatttacagatgaggaatggaatgaattgaaacacgattttatatcacaatatatacaaagtgaaccactgaatgtaccacaatatgatgtattaaaggagttaccaatgaatatagtaggtaatgttttagatgatggtataaacgaaaaaccttttattacttctattcatgatagggatttaaatagtggagaagaaattagttataatattaatatgagtactaatagtatggatgatccaaaatatgtatcaaataatgtatattctggtatagatttaattaatgattcactaagtggtggtaaacctattgatatatatgatgaagtgctaaaaagaaaagaaaatgaattatttggaacaaattataagaaaaatacatcaaataacaatgtagctaaattaacaaatagtgatccaattatgaaccaattagatttgttacataaatggttagatagacatagagatatgtgtgagatgtggaataataaagaggaagtattagataaattaaaagaacaatggaataaagataatgatggtggtgatatatcaagtgatagtaacaaaaggttgaatacggatgtttcgattgaaatagatatggatgatcctaaaggaaagaaggaatttagtaatatggatactatcttggataatatagaagatgatatatattatgatgtaaatgatgaaaacccatctgtgaatgatatacctatggatcataataaagtagatgtacctaagaaagtacatgttgaaatgaaaatccttaataatacatccactggatccttggaacaacaatttcctatatcggatgtatggaatatataa

SEQ ID NO: 6 - protein, 3346 aa, Plasmodium falciparum
MGNTQSSEEEEAKSPSLTESHNSARGVLEEIGKKIKDKTEKESKHVRQLKGKLSNAKFADRLYKESGGDLRSAYSDACSLTYKFHTNITTDGGDGRHPCHGRENNRFSESQEYGCSNVYIKGNENNSNGTACVPPRRRHICDQNLEFLDNPHTDDTDDLLGNVLVTAKYEGNYIVSNHPDKNSNGNKSGICTSLARSFADIGDIVRGRDMFKSNEKVEIGLKKVFEKINNGLKKIGINDYNDISGNYYKLREAWWTANRDQVWKAITCRAPNGANYFRKGLDGKIIFSDNGPCGRKELIVPTYLDYVPQFLRWLNEWSEEFCRIKNIKIGNIKKSCTGESNNKHCSREGYDCNKTNLRLNEIFMDLECPRCADDCKSYETWVEKKKKEFNKQKKKYEKEVDATQNNDNNENGIYNKKFYDELKSSYKEVNSFFELLNKGPICEHIDKKIPMDYNNTEKTFSRSEYCKSCPITDILCDDNECKTINEFKCREIKSMPNIRKNENETPIDIDILVNVNNKKVITHDLKNNYENCDLFKKLGEQKWKCKYKCYLDVCEPRNLDSNIYNERYISIKVLFKRWLEYFLEDYNKLKEKLNPCMYNVQEIVCINECKQNCECVEKWIKEKREEWKKIKDRYVQQYESKDEDVSSKLKKFLKQELFTNYVKNALDKDETLDSMKESTECIDPNKPKGKPCNNNDVINILLNRLEKQIDNCKKKHEEKGEKPCVDIPKLLNDEDEDEDDDETPRAHNPCVDKNDSQPTKTVSYIARQMHRRAKAQMTKNSVVDGDNKLEGDIFKVTFRNGGVGKNLNGDICKIDKTYSNDSRGTPTDGPCEGKGDRFKIGTDWQGDSFVNPQYRGIYMPPRRQHFCTSNLEKLDVSRVIRNGNASNSLLGDVLLAAKYEAERTKNHYVSKKEEHSEACRAVRYSFADLGDIIRGKDMWDKNHGEKKTQENLERIFAKIKEQLLNSSIKDKYKDDDKATPKYKQLREDWWEANRSQVWEAMQCPPKNGTFPCKSDHTPLHDYIPQRLRWMTEWAEWYCKEQSRLYGELVETCGKCMHKGKCKQGNGHCVTCKPACEKYKKFINTWQPQWKQMEQKYSQLYEEAKKYNDSSRKDTTNKDDYVLQFLNKLLTQNKGNKTYDTAEGYVHQEAHISDCQKQTQFCKKRNGEIPSSDTETDNNYAFRPQPHDHDEVCECNTRQKTKVRKKKKKVDACEMAKTLLHNNDGTIRIGQCKRKDEGNAEYPKWDCNSQIHTTHNGACMPPRRQKLCVYFFANPSQIGSINKQDNLRKAFIISAAAETFRSWQYYKSKNGGENLQTQLKDGTIPDDFKRQMFYTYGDYRDFLFGTDISKGLGEGTALEKQINILFPNGVRKIPNEKTREKWWTDHGPEIWKGMLCALTNGLSESEKKTKIFDDYSHDKVNQSKNGNPSLEDFAKKPQFFRWFIEWSDEFCRERKKKEEEVERDCKDEYEGCEKEKNGKCVTACKAYKEYITNKKEEYDSQKGKFDVEKTEKKQGYEDYSEKQASEYLKEKCIKSSCNCMKKVTEISNYWTNPHKTYDTENLGIKCECPPSPCTIVDGILSPQNSSSYAEGCKWKYGKMSQGGTEWDCSKKSGGEGGNEDGDVVCIPPRRRRLYVKNLQDLTGEESLVDLRKAFIKCAAIETFFAWHEFKKEKEREEKEKNEQDVQYKSSVLENLQKQLKNGEIDDEFKRQMFYTFADYRDICLGKDIGNDVDGINEKIDTILQKNGKPNNIEEYKKWWQKHGHEIWEGMLCALSYNTETKEMDKELRNKLTEQKNGNKNTYDTVTISGGPIGNTKLEKFASRPPFFRWLEEWADEFCRKRTHKLEKIQNECKGVSGTNQCDDDGFDCDEMCPKKDGSFETFKCLSCAKSCRFYKKWISRKKEEFDKQSKKYENEIDDVKHNSDNIYGKDFLETLDQQYKSVELFLEKVKGPCSINNNNEECKIDFNKPKDTFGHAKNCGPCSEIRFKCIEDNSNWVTTNTCNKTTFKFTEDNKDTKEDSEQLGMLISDNTVQNFADGLQNDCKDADIFKGLRKDQWSCGYFCNLDICSLKTSHGENNYKQNILIRALFKRWLEHFLEDYNKINDKISHCMKNGEGSTCIKGCEIKCNCVSNWIKKKTLEWEIVRDRFFKQYNVDSEKSFTVKSFLEQAPFDSDVQKAIKPFEKLRDFEDSIVCNGTTSARKEKGTEKDVVICLLDKLQKQIETCQTKHKETSGNTCSPPPNPDTQTDTPLPLESFPPPFCNVPPNPCGDKDATNVVGVEVLAKEMQEAAHKSMLSRSAVDSGKGDKGESSSGKSSLEGDISLAEFKNGFNPSGLKNVCQITEKHSYANGASKDPCNGKGNGKDQRFKIETQWKDTGKSGKHVDVYLPPRREHICTSNLEYLLKGNSDQIMKVGNNKINHSFLGEVLLAAKYEAEFIKTNYTRLNGQNDNGAKCRAMKYSFADIGDIVRGRDLWEHNDFKKLERDLVKIFGKIKEGITDETTKKQYEKDDTDNKQLRCDWWEANRDQVWEAMQCKTTIPPVTTSCDTTTVTPLVDYIPQRLRWMMEWAEWYCKYQSKAYSELRKGCEDCRSWKCMKGDSKCENCTKACKDYNSKIEPWKQQWTKIKEKYEELYKKAQNSDTSNSGTTYPKDEKDVVSFLSKLHEKNKDNKIYYTAAGYIHQQAKYLDCTQQTHFCDKKNGETLPSGRDNDKYAFKKPPKKYERACKCHEKQEPPPPKVPEDSEDDRERSEPGEDALPVLPPEEIEQEEEPEETSVDTTQDEEEPASEGGGPSGSPTEESGEPRENSDSSDPKPDQNPEANPEQTPILKPEEEAPPKSKPPDGDRGVGRSLGPTPRSEVEPEESENEDVEDEDDEEEEEDPDDDPEAESEEEDEDHGGQEAEAVPPQPQAPAPLPPPPPPLPPLKTALMSSTIMWSVGIGFAAISYFLLKKKPKSPVDLIRVIDIHKGDYGIPTLESKNRYIPYVSDTYKGKTYIYMEGDTSGDEKYGFMSDTTDITSSESEYEELDINDIYVPGSPKYKTLIEVVLEPSKSNGNTLGDDMVPTTNTFTDEEWNELKHDFISQYIQSEPLNVPQYDVLKELPMNIVGNVLDDGINEKPFITSIHDRDLNSGEEISYNINMSTNSMDDPKYVSNNVYSGIDLINDSLSGGKPIDIYDEVLKRKENELFGTNYKKNTSNNNVAKLTNSDPIMNQLDLLHKWLDRHRDMCEMWNNKEEVLDKLKEQWNKDNDGGDISSDSNKRLNTDVSIEIDMDDPKGKKEFSNMDTILDNIEDDIYYDVNDENPSVNDIPMDHNKVDVPKKVHVEMKILNNTSTGSLEQQFPISDVWNI